SEQUENCE LISTING (303 sequences)

SEQ ID NO: 1-11 - skipped (11 entries)

SEQ ID NO: 12 - protein, 17 aa, synthetic construct
  VARIANT 6: note = N or Q
  VARIANT 13: note = Q or E
EINPIXGRSNYAXKFQG

SEQ ID NO: 13 - protein, 11 aa, synthetic construct
  VARIANT 8: note = N or S
RTSENIYXNLA

SEQ ID NO: 14 - protein, 7 aa, synthetic construct
  VARIANT 2: note = A or G
  VARIANT 7: note = D or E
AXTNLAX

SEQ ID NO: 15 - protein, 10 aa, synthetic construct
  VARIANT 10: note = May be deleted
QHFWGTPLTF

SEQ ID NO: 16 - protein, 7 aa, synthetic construct
  VARIANT 7: note = D or E
AATNLAX

SEQ ID NO: 17 - protein, 9 aa, synthetic construct
YTFTNYWMH

SEQ ID NO: 18 - protein, 17 aa, synthetic construct
EINPINGRSNYAQKFQG

SEQ ID NO: 19 - protein, 7 aa, synthetic construct
GTRAMHY

SEQ ID NO: 20 - protein, 17 aa, synthetic construct
EINPINGRSNYAEKFQG

SEQ ID NO: 21 - protein, 17 aa, synthetic construct
EINPIQGRSNYAEKFQG

SEQ ID NO: 22 - protein, 11 aa, synthetic construct
RTSENIYNNLA

SEQ ID NO: 23 - protein, 7 aa, synthetic construct
AATNLAD

SEQ ID NO: 24 - protein, 9 aa, synthetic construct
QHFWGTPLT

SEQ ID NO: 25 - protein, 7 aa, synthetic construct
AATNLAE

SEQ ID NO: 26 - protein, 10 aa, synthetic construct
QHFWGTPLTF

SEQ ID NO: 27 - protein, 11 aa, synthetic construct
RTSENIYSNLA

SEQ ID NO: 28 - protein, 7 aa, synthetic construct
AGTNLAD

SEQ ID NO: 29 - protein, 116 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSS

SEQ ID NO: 30 - protein, 116 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSS

SEQ ID NO: 31 - protein, 116 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSS

SEQ ID NO: 32 - protein, 116 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSS

SEQ ID NO: 33 - protein, 116 aa, synthetic construct
QVQLQQPGAELVKPGASVKLSCKASGYTFTNYWMHWVKQRPGQGLEWIGEINPINGRSNYGERFKTKATLTVDKSSSTAYMQLSSLTSEDSAVYYCARGTRAMHYWGQGTSVTVSS

SEQ ID NO: 34 - protein, 107 aa, synthetic construct
DIQMTQSPSSLSASVGDRVTITCRTSENIYNNLAWYQQKPGKSPKLLIYAATNLADGVPSRFSGSGSGTDYTLTISSLQPEDFATYYCQHFWGTPLTFGGGTKVEIK

SEQ ID NO: 35 - protein, 107 aa, synthetic construct
DIQMTQSPSSLSASVGDRVTITCRTSENIYNNLAWYQQKPGKAPKLLIYAATNLADGVPSRFSGSGSGTDYTLTISSLQPEDFATYYCQHFWGTPLTFGGGTKVEIK

SEQ ID NO: 36 - protein, 107 aa, synthetic construct
DIQMTQSPSSLSASVGDRVTITCRTSENIYNNLAWYQQKPGKAPKLLIYAATNLAEGVPSRFSGSGSGTDYTLTISSLQPEDFATYYCQHFWGTPLTFGGGTKVEIK

SEQ ID NO: 37 - protein, 107 aa, synthetic construct
DIQMTQSPSSLSASVGDRVTITCRTSENIYSNLAWYQQKPGKAPKLLIYAGTNLADGVPSRFSGSGSGTDYTLTISSLQPEDFANYYCQHFWGTPLTFGGGTKVEIK

SEQ ID NO: 38 - protein, 107 aa, synthetic construct
DIQMTQSPASLSVSVGETVTITCRTSENIYNNLAWYQQKQGKSPQLLVYAATNLADGVPSRFSGSGSGTQYSLKINSLQSEDFGNYYCQHFWGTPLTFGAGTKLELK

SEQ ID NO: 39 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 41 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCGVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 42 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 43 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 45 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 46 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCGVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 49 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 50 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 51 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 52 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCGVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 55 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 57 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 58 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 59 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCGVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 60 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 61 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 62 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 63 - protein, 214 aa, synthetic construct
DIQMTQSPSSLSASVGDRVTITCRTSENIYNNLAWYQQKPGKSPKLLIYAATNLADGVPSRFSGSGSGTDYTLTISSLQPEDFATYYCQHFWGTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 214 aa, synthetic construct
DIQMTQSPSSLSASVGDRVTITCRTSENIYNNLAWYQQKPGKAPKLLIYAATNLADGVPSRFSGSGSGTDYTLTISSLQPEDFATYYCQHFWGTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 214 aa, synthetic construct
DIQMTQSPSSLSASVGDRVTITCRTSENIYNNLAWYQQKPGKAPKLLIYAATNLAEGVPSRFSGSGSGTDYTLTISSLQPEDFATYYCQHFWGTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 214 aa, synthetic construct
DIQMTQSPSSLSASVGDRVTITCRTSENIYSNLAWYQQKPGKAPKLLIYAGTNLADGVPSRFSGSGSGTDYTLTISSLQPEDFANYYCQHFWGTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67-79 - skipped (13 entries)

SEQ ID NO: 80 - DNA, 27 bp, synthetic construct
  source 1..27: mol_type = other DNA
gttcttctagccttaagataccatttg

SEQ ID NO: 81 - DNA, 27 bp, synthetic construct
  source 1..27: mol_type = other DNA
gttcttctagccttaagataccatttg

SEQ ID NO: 82 - DNA, 29 bp, synthetic construct
  source 1..29: mol_type = other DNA
acacaaattcctgagaattgggaacatgc

SEQ ID NO: 83 - DNA, 20 bp, synthetic construct
  source 1..20: mol_type = other DNA
cagtggctaacagaagctga

SEQ ID NO: 84 - DNA, 27 bp, synthetic construct
  source 1..27: mol_type = other DNA
gttcttctagccttaagataccatttg

SEQ ID NO: 85 - DNA, 29 bp, synthetic construct
  source 1..29: mol_type = other DNA
acacaaattcctgagaattgggaacatgc

SEQ ID NO: 86 - DNA, 19 bp, synthetic construct
  source 1..19: mol_type = other DNA
agatctgttgagaaatggc

SEQ ID NO: 87 - DNA, 20 bp, synthetic construct
  source 1..20: mol_type = other DNA
tgttcttctagccttaagat

SEQ ID NO: 88 - DNA, 26 bp, synthetic construct
  source 1..26: mol_type = other DNA
tcagtggctaacagaagctgaacagt

SEQ ID NO: 89 - DNA, 18 bp, synthetic construct
  source 1..18: mol_type = other DNA
agggtgaagctacaggaa

SEQ ID NO: 90 - DNA, 20 bp, synthetic construct
  source 1..20: mol_type = other DNA
ttgttcttctagcccttgtc

SEQ ID NO: 91 - DNA, 27 bp, synthetic construct
  source 1..27: mol_type = other DNA
tctctcccagcttgatttccaatggga

SEQ ID NO: 92 - DNA, 22 bp, synthetic construct
  source 1..22: mol_type = other DNA
tggttggaggaagcagataaca

SEQ ID NO: 93 - DNA, 19 bp, synthetic construct
  source 1..19: mol_type = other DNA
taggaaaaggctagaagaa

SEQ ID NO: 94 - DNA, 24 bp, synthetic construct
  source 1..24: mol_type = other DNA
cacaaattcctgagaattgggaac

SEQ ID NO: 95 - DNA, 24 bp, synthetic construct
  source 1..24: mol_type = other DNA
tcatccaggttgtgataaacatct

SEQ ID NO: 96 - DNA, 26 bp, synthetic construct
  source 1..26: mol_type = other DNA
tggtatcttaaggacctccaaggtga

SEQ ID NO: 97-99 - skipped (3 entries)

SEQ ID NO: 100 - DNA, 30 bp, synthetic construct
  source 1..30: mol_type = other DNA
tttgccgctgcccaatgccatcctggagtt

SEQ ID NO: 101 - DNA, 30 bp, synthetic construct
  source 1..30: mol_type = other DNA
gtttgccgctgcccaatgccatcctggagt

SEQ ID NO: 102 - DNA, 30 bp, synthetic construct
  source 1..30: mol_type = other DNA
agtttgccgctgcccaatgccatcctggag

SEQ ID NO: 103 - DNA, 30 bp, synthetic construct
  source 1..30: mol_type = other DNA
cagtttgccgctgcccaatgccatcctgga

SEQ ID NO: 104 - DNA, 30 bp, synthetic construct
  source 1..30: mol_type = other DNA
caacagtttgccgctgcccaatgccatcct

SEQ ID NO: 105 - DNA, 30 bp, synthetic construct
  source 1..30: mol_type = other DNA
acaacagtttgccgctgcccaatgccatcc

SEQ ID NO: 106 - DNA, 30 bp, synthetic construct
  source 1..30: mol_type = other DNA
gacaacagtttgccgctgcccaatgccatc

SEQ ID NO: 107 - DNA, 30 bp, synthetic construct
  source 1..30: mol_type = other DNA
tgacaacagtttgccgctgcccaatgccat

SEQ ID NO: 108 - DNA, 30 bp, synthetic construct
  source 1..30: mol_type = other DNA
ctgacaacagtttgccgctgcccaatgcca

SEQ ID NO: 109 - DNA, 30 bp, synthetic construct
  source 1..30: mol_type = other DNA
tctgacaacagtttgccgctgcccaatgcc

SEQ ID NO: 110 - DNA, 20 bp, synthetic construct
  source 1..20: mol_type = other DNA
tgccgctgcccaatgccatc

SEQ ID NO: 111 - DNA, 21 bp, synthetic construct
  source 1..21: mol_type = other DNA
ttgccgctgcccaatgccatc

SEQ ID NO: 112 - DNA, 22 bp, synthetic construct
  source 1..22: mol_type = other DNA
tttgccgctgcccaatgccatc

SEQ ID NO: 113 - DNA, 23 bp, synthetic construct
  source 1..23: mol_type = other DNA
gtttgccgctgcccaatgccatc

SEQ ID NO: 114 - DNA, 24 bp, synthetic construct
  source 1..24: mol_type = other DNA
agtttgccgctgcccaatgccatc

SEQ ID NO: 115 - DNA, 25 bp, synthetic construct
  source 1..25: mol_type = other DNA
cagtttgccgctgcccaatgccatc

SEQ ID NO: 116 - DNA, 26 bp, synthetic construct
  source 1..26: mol_type = other DNA
acagtttgccgctgcccaatgccatc

SEQ ID NO: 117 - DNA, 27 bp, synthetic construct
  source 1..27: mol_type = other DNA
aacagtttgccgctgcccaatgccatc

SEQ ID NO: 118 - DNA, 28 bp, synthetic construct
  source 1..28: mol_type = other DNA
caacagtttgccgctgcccaatgccatc

SEQ ID NO: 119 - DNA, 29 bp, synthetic construct
  source 1..29: mol_type = other DNA
acaacagtttgccgctgcccaatgccatc

SEQ ID NO: 120-199 - skipped (80 entries)

SEQ ID NO: 200 - RNA, 176 nt, Homo sapiens
  source 1..176: mol_type = mRNA
gaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagag

SEQ ID NO: 201-299 - skipped (99 entries)

SEQ ID NO: 300 - protein, 4 aa, synthetic construct
GGFG

SEQ ID NO: 301 - protein, 4 aa, synthetic construct
ALAL

SEQ ID NO: 302 - protein, 4 aa, synthetic construct
GFLG

SEQ ID NO: 303 - DNA, 24 bp, synthetic construct
  source 1..24: mol_type = other DNA
gaggcagattactgtggattctga

(19) United States
(12) Patent Application Publication
(10) Pub. No.: US 2025/0121085 A1
(43) Pub. Date: Apr. 17, 2025

(54) ANTIBODY-OLIGONUCLEOTIDE CONJUGATE COMPOSITIONS AND METHODS OF INDUCING DMD EXON 45 SKIPPING

(71) Applicant: Avidity Biosciences, Inc., San Diego, CA (US)

(72) Inventors: Beatrice Diana DARIMONT, San Diego, CA (US); Usue ETXANIZ IRIGOIEN, San Diego, CA (US); Venkata Ramana DOPPALAPUDI, San Diego, CA (US); Michael Caramian COCHRAN, La Jolla, CA (US); Isaac MARKS, San Diego, CA (US); Tyler ALBIN, La Jolla, CA (US)

(21) Appl. No.: 18/903,935

(22) Filed: Oct. 1, 2024

Related U.S. Application Data
(60) Provisional application No. 63/587,394, filed on Oct. 2, 2023.

(51) Int. Cl. A61K 47/68; A61P 21/00; C07K 16/28

(52) U.S. Cl. CPC A61K 47/6807; A61K 47/6849; A61K 47/6889; A61P 21/00; C07K 16/2881

(57) ABSTRACT

Disclosed herein are binding moiety (e.g., antibody)-oligonucleotide (e.g., PMO) conjugates and pharmaceutical compositions that induce an alteration in an incorrectly spliced dystrophin mRNA transcript to induce DMD exon 45 skipping. Also described herein include methods for treating muscle dystrophy including Duchenne muscular dystrophy that comprises administering such conjugates or a pharmaceutical composition that induces alteration in an incorrectly spliced dystrophin mRNA transcript to induce DMD exon 45 skipping.

### CROSS REFERENCE

This application claims the benefit of U.S. Provisional Application No. 63/587,394, filed Oct. 2, 2023, which is incorporated herein by reference in its entirety.

### REFERENCE TO A SEQUENCE LISTING XML

This application contains a Sequence Listing which has been submitted electronically in XML format. The Sequence Listing XML is incorporated herein by reference. Said XML file, created on Oct. 15, 2024, is named 45532-776_201_SL.xml and is 141,870 bytes in size.

### BACKGROUND OF THE DISCLOSURE

Duchenne Muscular Dystrophy (DMD) is a rare X-linked neuromuscular disease that manifests primarily in boys, affecting about 1:5000-10,000 males born worldwide. There are about 300,000 DMD patients worldwide. DMD is a monogenic disease; it is progressive, severe and irreversible. The disease is caused by mutations in the DMD gene, the longest gene in the human genome (79 exons), which encodes for the dystrophin protein (430 kDa). The central domain of dystrophin, called rod domain, is formed by 24 spectrin repeats that function as a shock-absorber and protect the sarcolemma from damage during movement.

DMD is caused by mutations (changes) within the dystrophin gene. Deletions of one or more exons are the most common type of mutation. Since there are a total of 79 exons in the dystrophin gene, there are many different deletions that can occur. However, there are certain areas of the gene that are more likely to have a deletion, and these areas are called “hot spots”. The deletions in the DMD gene that are non-randomly distributed with many of the large gene deletions that occur in the DMD gene can be detected in specific hotspot areas of the gene. These hotspots are clustered within two main regions: about 20% of the deletions occur at the 5′ proximal portion of the gene (exons 1, 3, 4, 5, 8, 13, 19); and about 80% of the deletions occur at the mid-distal region i.e. 42-45, 47, 48, 50-53 (Den Dunnen et al. Am J Hum Genet. 1989; 45(6):835-847). The mutated DMD gene fails to produce any functional dystrophin and lack of functional dystrophin results in progressive muscle weakness due to muscle injury, repair, inflammation changes and paralysis.

Current research for DMD therapy includes stem cell replacement therapy, analog up-regulation, gene replacement, and exon-skipping technology. Exon-skipping technology uses structural analogs of DNA called antisense oligonucleotides to help cells skip over a specific exon during RNA splicing. These antisense oligonucleotides allow faulty parts of the dystrophin gene to be skipped over when it is transcribed to RNA for protein production, permitting a still-truncated but more functional version of the dystrophin protein to be produced by the muscle cells.

There are several antisense oligonucleotides that have already been approved for DMD patients with amenable to exon 45, 51, or 53 skipping. The antisense oligonucleotide named Eteplirsen has been approved in the United States for the treatment of mutations amenable to dystrophin exon 51 skipping. The antisense oligonucleotide named Golodirsen was approved for medical use in the United States in 2019, for the treatment of cases that can benefit from skipping exon 53 of the dystrophin transcript. The antisense oligonucleotide named Casimersen was approved for treatment in the United States in February 2021 for patients who have a confirmed mutation of the DMD gene that is amenable to exon 45 skipping.

Despite extensive research using exon skipping for exon 45 (U.S. Pat. Nos. 9,447,417, 8,461,325, and 8,361,979), there is only one recently FDA approved exon skipping therapy for DMD patients amenable to exon 45 skipping. Approximately 8% of the DMD patient population are amenable to exon 45 skipping and the majority of these DMD patients may also have a deletion of exon 44 of the DMD transcript.

A new class of therapeutics called antibody oligonucleotide conjugates (AOC) improves the delivery of antisense oligonucleotides. These AOCs target and deliver antisense oligonucleotides to specific tissue and cell types including muscle cells. These AOCs are being developed for the potential breakthrough therapy for DMD patients including patients that are amenable to exon 45 skipping. There is a need to provide improved therapy for DMD patients amenable to exon 45 skipping.

### SUMMARY OF THE DISCLOSURE

Disclosed herein, in certain aspects, is an oligonucleotide conjugate comprising a binding moiety for delivering to a muscle cell conjugated to an oligonucleotide molecule, wherein the oligonucleotide molecule comprises a nucleic acid sequence of at least 25 consecutive nucleotides from one of SEQ ID NOs: 100-109. In some instances, the oligonucleotide molecule is a phosphorodiamidate morpholino oligonucleotide (PMO) molecule. In some instances, the oligonucleotide molecule has 26-29 nucleotides in length. In some instances, the oligonucleotide molecule consists of a sequence from one of SEQ ID NOs: 116-119. In some instances, the binding moiety binds to a transferrin receptor on the muscle cell. In some instances, the binding moiety is an antibody or antigen binding fragment thereof. In some instances, the antibody or antigen binding fragment thereof comprises a humanized antibody or antigen binding fragment thereof, chimeric antibody or antigen binding fragment thereof, monoclonal antibody or antigen binding fragment thereof, monovalent Fab′, divalent Fab2, single chain variable fragment (scFv), diabody, minibody, nanobody, single domain antibody (sdAb), or camelid antibody or antigen binding fragment thereof. In some instances, the antibody or antigen binding fragment thereof is an anti-transferrin receptor antibody or antigen binding fragment thereof.

In some instances, the oligonucleotide molecule is conjugated to the binding moiety via a linker. In some instances, the linker is a cleavable linker, a non-cleavable linker, or is selected from the group consisting of a heterobifunctional linker, a homobifunctional linker, a maleimide group, a dipeptide moiety, a benzoic acid group or derivatives thereof, a C1-C6 alkyl group, and a combination thereof. In some instances, the oligonucleotide conjugate has a oligonucleotide-molecule-to-binding-moiety ratio of about 1:1, 2:1, 3:1, 4:1 5:1, 6:1, 7:1, 8:1 or higher or wherein the oligonucleotide conjugate has an average oligonucleotide-molecule-to-binding-moiety ratio of about 1, 2, 3, 4, 5, 6, 7, 8 or higher. In some instances, the oligonucleotide conjugate has a oligonucleotide-molecule-to-binding-moiety ratio of about 4:1 to 5:1, or wherein the oligonucleotide conjugate has an average oligonucleotide-molecule-to-binding-moiety ratio in the range of 4-5. In some instances, the oligonucleotide conjugate has a oligonucleotide-molecule-to-binding-moiety ratio of about 7:1 to 8:1, or wherein the oligonucleotide conjugate has an average oligonucleotide-molecule-to-binding-moiety ratio in the range of 7-8. In some instances, the oligonucleotide conjugate has a oligonucleotide-molecule-to-binding-moiety ratio of about 4:1 or 8:1 or wherein the oligonucleotide conjugate has an average oligonucleotide-molecule-to-binding-moiety ratio of about 4 or 8.

In certain aspects, provided herein is an oligonucleotide molecule that hybridizes to a pre-mRNA transcript of a DMD gene, wherein the oligonucleotide molecule comprises a nucleic acid sequence of at least 25 consecutive nucleotides from one of SEQ ID NOs: 100-109. In some instances, the oligonucleotide molecule is a phosphorodiamidate morpholino oligonucleotide (PMO) molecule. In some instances, the oligonucleotide molecule has 26-29 nucleotides in length. In some instances, wherein the oligonucleotide molecule consists of a sequence from one of SEQ ID NOs: 116-119.

In certain aspects, provided herein is a method of inducing exon 45 skipping in a subject in need thereof comprising administering to the subject an oligonucleotide molecule or an oligonucleotide conjugate comprising a binding moiety for delivering to a muscle cell conjugated to the oligonucleotide molecule comprising a nucleic acid sequence of at least 25 consecutive nucleotides from one of SEQ ID NOs: 100-109.

In certain aspects, provided herein is a method of generating a truncated dystrophin protein in a subject in need thereof comprising administering to the subject an oligonucleotide molecule or an oligonucleotide conjugate comprising a binding moiety for delivering to a muscle cell conjugated to the PMO molecule comprising a nucleic acid sequence of at least 25 consecutive nucleotides from one of SEQ ID NOs: 100-109.

In certain aspects, provided herein is a method of restoring dystrophin in a subject in need thereof comprising administering to the subject an oligonucleotide molecule or an oligonucleotide conjugate comprising a binding moiety for delivering to a muscle cell conjugated to the oligonucleotide molecule comprising a nucleic acid sequence of at least 25 consecutive nucleotides from one of SEQ ID NOs: 100-109.

In certain aspects, provided herein is a method of treating muscular dystrophy in a subject in need thereof comprising administering to the subject an oligonucleotide molecule or an oligonucleotide conjugate comprising a binding moiety for delivering to a muscle cell conjugated to the oligonucleotide molecule comprising a nucleic acid sequence of at least 25 consecutive nucleotides from one of SEQ ID NOs: 100-109.

In certain aspects, provided herein is a method of inducing exon 45 skipping in a targeted pre-mRNA transcript of DMD gene, comprising: a) contacting a muscle cell with an oligonucleotide molecule or an oligonucleotide conjugate comprising a binding moiety for delivering to a muscle cell conjugated to the oligonucleotide molecule comprising a nucleic acid sequence of at least 25 consecutive nucleotides from one of SEQ ID NOs: 100-109; b) hybridizing the oligonucleotide molecule to the targeted pre-mRNA transcript to induce exon 45 skipping in the targeted pre-mRNA transcript; and c) translating a mRNA transcript produced from the targeted pre-mRNA transcript processed in step b) in the muscle cell to generate a truncated dystrophin protein.

In some instances, the oligonucleotide molecule is a phosphorodiamidate morpholino oligonucleotide (PMO) molecule. In some instances, the oligonucleotide molecule has 26-29 nucleotides in length. In some instances, the oligonucleotide molecule consists of a sequence from one of SEQ ID NOs: 116-119. In some instances, the binding moiety binds to a transferrin receptor on the muscle cell. In some instances, the binding moiety is an antibody or antigen binding fragment thereof. In some instances, the antibody or antigen binding fragment thereof comprises a humanized antibody or antigen binding fragment thereof, chimeric antibody or antigen binding fragment thereof, monoclonal antibody or antigen binding fragment thereof, monovalent Fab′, divalent Fab2, single chain variable fragment (scFv), diabody, minibody, nanobody, single domain antibody (sdAb), or camelid antibody or antigen binding fragment thereof. In some instances, the antibody or antigen binding fragment thereof is an anti-transferrin receptor antibody or antigen binding fragment thereof. In some instances, the oligonucleotide molecule is conjugated to the binding moiety via a linker. In some instances, the linker is a cleavable linker, a non-cleavable linker, or is selected from the group consisting of a heterobifunctional linker, a homobifunctional linker, a maleimide group, a dipeptide moiety, a benzoic acid group or derivatives thereof, a C1-C6 alkyl group, and a combination thereof. In some instances, the oligonucleotide conjugate has a oligonucleotide-molecule-to-binding-moiety ratio of about 1:1, 2:1, 3:1, 4:1 5:1, 6:1, 7:1, 8:1 or higher or wherein the oligonucleotide conjugate has an average oligonucleotide-molecule-to-binding-moiety ratio of about 1, 2, 3, 4, 5, 6, 7, 8 or higher. In some instances, the oligonucleotide conjugate has a oligonucleotide-molecule-to-binding-moiety ratio of about 4:1 to 5:1, or wherein the oligonucleotide conjugate has an average oligonucleotide-molecule-to-binding-moiety ratio in the range of 4-5. In some instances, the oligonucleotide conjugate has a oligonucleotide-molecule-to-binding-moiety ratio of about 7:1 to 8:1, or wherein the oligonucleotide conjugate has an average oligonucleotide-molecule-to-binding-moiety ratio in the range of 7-8. In some instances, the oligonucleotide conjugate has a oligonucleotide-molecule-to-binding-moiety ratio of about 4:1 or 8:1 or wherein the oligonucleotide conjugate has an average oligonucleotide-molecule-to-binding-moiety ratio of about 4 or 8.

In some instances, the subject in need thereof is diagnosed with or to have a high/higher chance to develop a muscular dystrophy. In some instances, the muscular dystrophy is Duchenne muscular dystrophy or Becker muscular dystrophy.

### BRIEF DESCRIPTION OF THE DRAWINGS

FIG. 1 is a plot predicting human DMD exon 45 skipping activity of 25-mers and 30-mers phosphorodiamidate morpholino oligomers (PMOs). The top line represents the skipping activity of 30-mers. The bottom line represents the skipping activity of 25-mers. Small dots represent predicted data. Large dots represent experimental data.

FIG. 2 is a plot for the dose response curve of the relative levels of exon 45 skipping activity in response to increasing concentrations of 10 different 30-mer PMOs in human immortalized myoblasts.

FIG. 3 is a plot of the dose-response curve for the relative levels of exon 45 skipping activity in response to increasing concentrations of PMOs with 4 different lengths: hEx45_Ac9_20 (20-mer), hEx45_Ac9_25 (25-mer), hEx45_Ac9_28 (28-mer), and hEx45_Ac9_30 (30-mer).

FIGS. 4A-4B are bar graphs illustrating the relative levels of exon 45 skipping activity in response to 3 different concentrations of hEx45_Ac9_28 PMO (3 μM, 10 μM, 30 μM) in human primary myotubes. FIG. 4A is a bar graph plot of the relative levels of exon 45 skipping activity in response to the concentrations of 3 μM, 10 μM, 30 μM of hEx45_Ac9_28 PMO in primary myotubes from 3 different healthy donors (MB07, MB09 and W018). FIG. 4B is a bar graph plot of the relative levels of exon 45 skipping activity in response to the concentrations of 3 μM, 10 μM, 30 μM of hEx45_Ac9_28 in DMD primary myotubes derived from 3 different DMD patients (DMD del 46-55, DMD del. 18-44, and DMD del. 44).

FIGS. 5A-F are bar graphs illustrating the comparison for the relative levels of exon 45 skipping activity between the hEx45_Ac9_28 and the exon 45 positive PMO control at the concentrations of 3 μM, 10 μM or 30 μM in human primary myotubes from healthy donors and DMD patients. FIG. 5A-C are bar graph plots of the relative levels of exon 45 skipping activity in response to hEx45_Ac9_28 (PMO45-28mer) or the exon positive control at the concentration of 3 μM, 10 μM, or 30 μM in primary myotubes from 3 different healthy donors (MB07, MB09, and W018). FIG. 5D-F are bar graph plots of the relative levels of exon 45 skipping activity in response to hEx45_Ac9_28 (PMO45-28mer) or the exon 45 positive PMO control at the concentrations of 3 μM or 10 μM, 30 μM of in DMD primary myotubes derived from 3 different DMD patients (DMD del 46-55, DMD del. 18-44, and DMD del. 44).

FIGS. 6A-6B depict levels of dystrophin protein in response to increasing concentrations of hEx45_Ac9_28 in human primary myotubes derived from healthy donors and DMD patients. FIG. 6A illustrates pictures of immunofluorescence staining of dystrophin positive fibers in non-treated human primary myotubes from healthy donor and DMD primary myotubes transfected with hEx45_Ac9_28. FIG. 6B is a plot for the dose response curve of the relative levels of dystrophin in response to increasing concentrations of hEx45_Ac9_28 in DMD primary myotubes from 3 different DMD patients (DMD del 46-55, DMD del. 18-44, and DMD del. 44).

FIG. 7 is a bar graph illustrating the hEx45_Ac9_28 PMO concentrations in muscle and non-muscle tissues obtained from cynomolgus monkeys at day 42 that have been administered a single infusion of hEx45_Ac9_28 AOC at the dose of 122.3 mg/kg at day 0.

FIGS. 8A-8B are bar graphs illustrating the number of exon 45 skipped DMD copies in muscle and non-muscle tissues obtained from cynomolgus monkeys at day 42 that have been administered a single infusion of hEx45_Ac9_28 AOC at the dose of 123.3 mg/kg at day 0. FIG. 8A is a bar graph illustrating the number of exon 45 skipped copies in muscle and non-muscle tissues obtained from cynomolgus monkeys at day 42 that have been administered a single infusion of hEx45_Ac9_28 AOC at the dose of 122.3 mg/kg at day 0. FIG. 8B is a bar graph illustrating the number of exon 45 skipped copies in skeletal and cardiac muscle tissues obtained from cynomolgus monkeys at day 42 that have been administered a single infusion of hEx45_Ac9_28 AOC at the dose of 122.3 mg/kg at day 0.

FIG. 9 is a plot illustrating the binding assay of the DAR8 hEx45_Ac9_28 AOC or the unmodified anti-transferrin receptor monoclonal antibody to the transferrin receptor by ELISA.

FIGS. 10A-10B are bar graphs illustrating hEx45_Ac9_28 PMO tissue concentrations in skeletal muscles obtained from cynomolgus monkeys at day 7 that have been administered a single infusion of DAR4 hEx45_Ac9_28 AOC at the dose of 122.3 mg/kg at day 0 corresponding to the PMO (hEx45_Ac9-28) dose level of 30 mg/kg or DAR8 hEx45_Ac9_28 AOC at the dose of 93.3 mg/kg at day 0 corresponding to the PMO (hEx45 Ac9-28) dose level of 30 mg/kg. FIG. 10A is a bar graph illustrating tissue concentrations hEx45_Ac9_28 PMO in the gastrocnemius and vastus lateralis muscles obtained from cynomolgus monkeys at day 7 that have been administered a single infusion of DAR4 hEx45_Ac9_28 AOC at the dose of 122.3 mg/kg at day 0 or DAR8 hEx45_Ac9_28 AOC at the dose of 93.3 mg/kg at day 0. FIG. 10B is a bar graph illustrating relative tissue concentrations hEx45_Ac9_28 PMO normalized with antibody concentrations in the gastrocnemius and vastus lateralis muscles obtained from cynomolgus monkeys at day 7 that have been administered a single infusion of DAR4 hEx45_Ac9_28 AOC at the dose of 122.3 mg/kg at day 0 or DAR8 hEx45_Ac9_28 AOC at the dose of 93.3 mg/kg at day 0.

### DETAILED DESCRIPTION OF THE DISCLOSURE

Disclosed herein, in some aspects, are binding moiety (e.g., antibody)-polynucleic acid conjugate compositions for the treatment of muscle dystrophy. Also disclosed herein, in some aspects, are methods of treating muscle dystrophy caused by an incorrectly spliced DMD mRNA transcript in a subject in need thereof, the method comprising: administering to the subject a binding moiety (e.g., antibody)-polynucleic acid conjugate; wherein the binding moiety (e.g., antibody)-polynucleic acid conjugate induces alteration in the incorrectly spliced pre-mRNA dystrophy transcript to induce exon 45 skipping of the DMD mRNA transcript to generate a fully or partially processed DMD mRNA transcript; and wherein the fully or partially processed DMD mRNA transcript encodes a functional and truncated dystrophin protein, thereby treating the disease or disorder in the subject. As used herein, the term “polynucleic acid” is interchangeably used with the term “oligonucleotide”.

Disclosed herein, in some aspects, are binding moiety (e.g., antibody)-antisense oligonucleotide (ASO) conjugate or binding moiety (e.g., antibody)-Phosphorodiamidate morpholino oligomer (PMO) conjugate compositions for the treatment of muscle dystrophy. Also disclosed herein are methods of treating muscle dystrophy caused by an incorrectly spliced DMD mRNA transcript in a subject in need thereof, the method comprising: administering to the subject an binding moiety (e.g., antibody)-ASO conjugate or an binding moiety (e.g., antibody)-PMO conjugate; wherein the ASO or PMO induces alteration in the incorrectly spliced pre-mRNA dystrophy transcript to induce exon 45 skipping of the DMD mRNA transcript to generate a fully or partially processed DMD mRNA transcript; and wherein the fully or partially processed DMD mRNA transcript encodes a functional and truncated dystrophin protein, thereby treating the disease or disorder in the subject.

In some instances, one such area where binding moiety (e.g., antibody)-polynucleic acid conjugate is used is for treating muscular dystrophy. Muscular dystrophy encompasses several diseases that affect the muscle. Duchenne muscular dystrophy is a severe form of muscular dystrophy and caused by mutations in the DMD gene. In some instances, mutations in the DMD gene disrupt the translational reading frame and results in a non-functional dystrophin protein.

Described herein, in certain aspects, are methods and compositions relating to nucleic acid therapy to induce an insertion, deletion, duplication, or alteration in an incorrectly spliced mRNA transcript to induce exon skipping or exon inclusion, which is used to restore the translational reading frame. In some aspects, also described herein include methods and compositions for treating a disease or disorder characterized by an incorrectly processed mRNA transcript, in which after removal of an exon, the mRNA is capable of encoding a functional protein, thereby treating the disease or disorder. In additional aspects, described herein include pharmaceutical compositions and kits for treating the same.

### RNA Processing

RNA has a central role in regulation of gene expression and cell physiology. Proper processing of RNA is important for the translation of functional proteins. Alterations in RNA processing such as a result of incorrect splicing of RNA can result in disease. For example, mutations in a splice site causes exposure of a premature stop codon, a loss of an exon, or inclusion of an intron. In some instances, alterations in RNA processing results in an insertion, deletion, or duplication. In some instances, alterations in RNA processing results in an insertion, deletion, or duplication of an exon. Alterations in RNA processing, in some cases, results in an insertion, deletion, or duplication of an intron.

### Exon Skipping

As used herein, the term “pre-mRNA” refers to the product of transcription which is comprised of both exons (coding sequences) and introns (non-coding sequences). Exon skipping is a form of RNA splicing. In some cases, exon skipping occurs when an exon is skipped over the pre-mRNA transcript or is spliced out of the processed mRNA. As a result of exon skipping, the processed mRNA does not contain the skipped exon. In some instances, exon skipping results in expression of an altered transcript and/or mRNA product. For instance, exon 45 skipping occurs when exon 45 is skipped over in the pre-mRNA transcript or is spliced out of the processed DMD mRNA. As a result of the exon 45 skipping, the processed DMD mRNA does not contain the skipped exon 45. In some instances, exon 45 skipping results in the expression of a truncated dystrophin protein. In some instances, exon 45 skipping results in the expression of a functional dystrophin protein. In some instances, exon 45 skipping results in the expression of a truncated and functional dystrophin protein.

In some instances, oligonucleotide (e.g., PMO)-binding moiety (e.g., antibody) conjugates (e.g., PMO-AOC) are used to induce exon skipping. In some instances, oligonucleotide (e.g., PMO)-binding moiety (e.g., antibody) conjugates are used to deliver oligonucleotides (e.g., PMO) for inducing exon skipping (e.g., in a cell, preferably in a muscle cell, etc.). In some instances, the delivered oligonucleotides (e.g., PMO) are used to induce exon skipping. For example, the oligonucleotides (e.g., PMO) bind splice sites or exonic enhancers. In some instances, binding of oligonucleotides (e.g., PMO) to specific mRNA or pre-mRNA sequences generates double-stranded regions. In some instances, oligonucleotide (e.g., PMO)s bind to acceptor or donor splice site at the beginning and/or at the end of an exon. In some instances, oligonucleotides (e.g., PMO)-binding moiety (e.g., antibody) conjugates are used to induce exon 45 skipping. In some instances, oligonucleotides (e.g., PMO)-binding moiety (e.g., antibody) conjugates are used to deliver oligonucleotides (e.g., PMO) for inducing exon 45 skipping. The delivered oligonucleotides (e.g., PMO) are used to induce exon 45 skipping. For example, the delivered oligonucleotides (e.g., PMO) bind to at least one of splice sites or exonic enhancers of exon 45. In some instances, binding of oligonucleotides (e.g., PMO) to specific mRNA or pre-mRNA sequences generates double-stranded regions. In some instances, oligonucleotides (e.g., PMO) bind to acceptor or donor splice site at the beginning and/or at the end of exon 45. In some instances, oligonucleotides (e.g., PMO) bind to acceptor splice site at the beginning (e.g., 5′-end)/end (e.g., 3′-end) of exon 45. In some instances, oligonucleotides (e.g., PMO) bind to donor splice site at the beginning (e.g., 5′-end)/end (e.g., 3′-end) of exon 45. In some instances, antisense oligonucleotides (AONs, ASOs) are used to induce exon skipping. In some aspects, the polynucleic acid is an antisense oligonucleotide (ASO) or a PMO molecule. In some aspects, the binding moiety (e.g., antibody)-polynucleic acid conjugate is an ASO-binding moiety (e.g., antibody) conjugate. In some aspects, the binding moiety (e.g., antibody)-polynucleic acid conjugate is a PMO-binding moiety (e.g., antibody) conjugate. In some aspects, a PMO molecule of the PMO-binding moiety (e.g., antibody) conjugate described herein induces exon 45 skipping to induce an alteration in an incorrectly spliced mRNA transcript. In some instances, the oligonucleotide (e.g., PMO) molecule restores the translational reading frame of the dystrophin protein by altering the incorrectly spliced mRNA transcript. In some instances, the oligonucleotide (e.g., PMO) molecule results in a functional and truncated dystrophin protein by restoring the translational reading frame of the dystrophin protein.

In some aspects, a polynucleic acid molecule is conjugated to an antibody for delivery to a site of interest. In some cases, an oligonucleotide (e.g., PMO) molecule is conjugated to an antibody. In some cases, an oligonucleotide (e.g., PMO) molecule is conjugated to an antibody for delivery to a site of interest. In some aspects, an oligonucleotide (e.g., PMO) molecule is conjugated to an antibody for delivery to a muscle cell. In some cases, an oligonucleotide (e.g., PMO) molecule for skipping exon 45 is conjugated to an antibody. In some cases, an oligonucleotide (e.g., PMO) molecule for skipping exon 45 is conjugated to an antibody for delivery to a muscle cell.

In some instances, the oligonucleotide conjugate disclosed herein are delivered into skeletal muscle tissues. In some instances, the oligonucleotide conjugate disclosed herein are delivered into cardiac muscle tissues. In some instances, the oligonucleotide conjugate disclosed herein preferably induces exon skipping in muscle tissues. In some instances, the oligonucleotide conjugate disclosed herein preferably induces exon skipping in muscle tissues. In some instances, the oligonucleotide conjugate disclosed herein preferably generates truncated dystrophin proteins in muscle tissues. In some instances, the oligonucleotide conjugate disclosed herein preferably generates truncated dystrophin proteins in muscle tissues. In some instances, the oligonucleotide conjugate disclosed herein preferably generates truncated dystrophin proteins in muscle tissues. In some instances, the oligonucleotide conjugate disclosed herein preferably generates truncated dystrophin proteins in muscle tissues. In some instances, the oligonucleotide conjugate disclosed herein preferably restores dystrophin in muscle tissues. In some instances, the oligonucleotide conjugate disclosed herein preferably restores dystrophin in muscle tissues.

In some instances, an antibody is conjugated to at least one oligonucleotide (e.g., PMO) molecule. In some instances, the antibody is conjugated to the at least one oligonucleotide (e.g., PMO) molecule to form an oligonucleotide (e.g., PMO)-binding moiety (e.g., antibody) conjugate. In some aspects, the antibody is conjugated to the 5′ terminus of the oligonucleotide (e.g., PMO) molecule, the 3′ terminus of the oligonucleotide (e.g., PMO) molecule, an internal site on the oligonucleotide (e.g., PMO) molecule, or in any combinations thereof. In some instances, the antibody is conjugated to at least two oligonucleotide (e.g., PMO) molecules. In some instances, the antibody is conjugated to at least 2, 3, 4, 5, 6, 7, 8, or more oligonucleotide (e.g., PMO) molecules.

As used herein, the term “AONs” is interchangeably used with the term “ASOs” and both refer to antisense oligonucleotides. In some instances, AONs are short nucleic acid sequences that bind to specific mRNA or pre-mRNA sequences. For example, AONs bind to splice sites or exonic enhancers. In some instances, binding of AONs to specific mRNA or pre-mRNA sequences generates double-stranded regions. In some instances, formation of double-stranded regions occurs at sites where the spliceosome or proteins associated with the spliceosome would normally bind to and causes exons to be skipped. In some instances, skipping of exons results in restoration of the transcript reading frame and allows for production of an at least partially functional dystrophin protein.

### Oligonucleotide Molecules

In some instances, an oligonucleotide (e.g., PMO) molecule (e.g., PMO of the PMO-antibody conjugate) targets and hybridizes to a pre-mRNA sequence of the DMD gene. In some instances, the oligonucleotide (e.g., PMO) molecule targets and hybridizes a splice site of exon 45 of the pre-mRNA sequence of the DMD gene. In some instances, the oligonucleotide (e.g., PMO) molecule targets and hybridizes a cis-regulatory element of exon 45 of the pre-mRNA sequence of the DMD gene. In some instances, the oligonucleotide (e.g., PMO) molecule targets and hybridizes a trans-regulatory element of exon of the pre-mRNA sequence of the DMD gene. In some instances, the oligonucleotide (e.g., PMO) molecule targets exonic splice enhancers or intronic splice enhancers of exon 45 of the pre-mRNA sequence of the DMD gene. In some instances, the oligonucleotide (e.g., PMO) molecule targets and hybridizes exonic splice silencers or intronic splice silencers of exon 45 of the pre-mRNA sequence of the DMD gene. In some instances, the oligonucleotide (e.g., PMO) molecule targets and hybridizes to the acceptor site of exon 45 of the pre-mRNA sequence of the DMD gene.

In some instances, an oligonucleotide (e.g., PMO) molecule (e.g., PMO of the PMO-antibody conjugate) targets and hybridizes a sequence found in introns or exons of the pre-mRNA sequence of the DMD gene. For example, the oligonucleotide (e.g., PMO) molecule targets and hybridizes to a sequence found in exon 45 of the pre-mRNA sequence of the DMD gene that mediates splicing of said exon. In some instances, the oligonucleotide (e.g., PMO) molecule targets an exon recognition sequence of the pre-mRNA sequence of the DMD gene. In some instances, the oligonucleotide (e.g., PMO) molecule targets a sequence upstream of an exon of the pre-mRNA sequence of the DMD gene. In some instances, the oligonucleotide (e.g., PMO) molecule targets a sequence downstream of an exon of the pre-mRNA sequence of the DMD gene.

As described above, an oligonucleotide (e.g., PMO) molecule targets an incorrectly processed mRNA transcript which results in a neuromuscular disease or disorder. In some cases, a neuromuscular disease or disorder is Duchenne muscular dystrophy or Becker muscular dystrophy.

In some instances, the oligonucleotide (e.g., a PMO molecule, an antisense oligonucleotide, etc.) targets a region (a sequence) adjacent to a mutated exon. In another instance, if there is a mutation in exon 45, the polynucleic acid molecule targets a sequence in exon 45 (e.g., a region within exon 45) of the pre-mRNA sequence of the DMD gene so that exon 45 is skipped.

In some cases, an oligonucleotide described herein targets a region that is at the exon-intron junction of exon 45 of the pre-mRNA sequence of the DMD gene. In some cases, a polynucleic acid molecule described herein targets a region that is at the exon-intron junction of exon 45 of the pre-mRNA sequence of the DMD gene.

In some instances, the oligonucleotide (e.g., PMO) molecule (e.g., PMO of the PMO-antibody conjugate) conjugate hybridizes to a target region that is at either the 5′ intron-exon junction or the 3′ exon-intron junction of exon 45 of the pre-mRNA of the DMD gene.

In some cases, the polynucleic acid molecule hybridizes to a target region that is at the 5′ intron-exon junction of exon 45 of the pre-mRNA of the DMD gene.

In some cases, the oligonucleotide (e.g., PMO) molecule hybridizes to a target region that is at the 3′ exon-intron junction of exon 45 of the pre-mRNA of the DMD gene.

In some instances, an oligonucleotide (e.g., PMO) molecule of the oligonucleotide (e.g., PMO of the PMO-antibody conjugate) described herein targets a splice site of exon 45 of the pre-mRNA of the DMD gene. In some cases, an oligonucleotide (e.g., PMO of the PMO-antibody conjugate) described herein targets a splice site of exon 45 of the pre-mRNA of the DMD gene. As used herein, a splice site includes a canonical splice site, a cryptic splice site or an alternative splice site that is capable of inducing an insertion, deletion, duplication, or alteration in an incorrectly spliced mRNA transcript to induce exon 45 skipping.

In some instances, the oligonucleotide (e.g., PMO of the PMO-antibody conjugate) hybridizes to a target region that is proximal to the exon-intron junction. In some instances, an oligonucleotide (e.g., PMO) molecule described herein targets a region at least 1000 nucleotides (nt), 500 nt, 400 nt, 300 nt, 200 nt, 100 nt, 80 nt, 60 nt, 50 nt, 40 nt, 30 nt, 20 nt, 10 nt, or 5 nt upstream (or the 5′) of exon 45 of the pre-mRNA of the DMD gene. In some instances, an oligonucleotide (e.g., PMO) molecule described herein targets a region at least 1000 nt, 500 nt, 400 nt, 300 nt, 200 nt, 100 nt, 80 nt, 60 nt, 50 nt, 40 nt, 30 nt, 20 nt, 10 nt, or 5 nt upstream (or 5′) of exon 45 of the pre-mRNA of the DMD gene.

In some instances, the oligonucleotide (e.g., PMO of the PMO-antibody conjugate) hybridizes to a target region that is downstream (or 3′) to exon 45 of the pre-mRNA of the DMD gene. In some instances, the polynucleic acid molecule hybridizes to a target region that is about 5, 10, 15, 20, 50, 100, 200, 300, 400 or 500 nt downstream (or 3′) to exon 45 of the pre-mRNA of the DMD gene.

In some instances, an oligonucleotide (e.g., PMO of the PMO-antibody conjugate) described herein targets an internal region within exon 45 of the pre-mRNA of the DMD gene.

In some aspects, the oligonucleotide (e.g., PMO of the PMO-antibody conjugate) described herein targets a partially spliced mRNA sequence comprising exon 45 of the pre-mRNA of the DMD gene. In some instances, the oligonucleotide (e.g., PMO) molecule hybridizes to a target region that is upstream (or 5′) to exon 45 of the pre-mRNA of the DMD gene. In some instances, the oligonucleotide (e.g., PMO) molecule hybridizes to a target region that is about 5, 10, 15, 20, 50, 100, 200, 300, 400 or 500 bp upstream (or 5′) to exon 45 of the pre-mRNA of the DMD gene. In some instances, the oligonucleotide (e.g., PMO) molecule hybridizes to a target region that is downstream (or 3′) to exon 45 of the pre-mRNA of the DMD gene. In some instances, the oligonucleotide (e.g., PMO) molecule hybridizes to a target region that is about 5, 10, 15, 20, 50, 100, 200, 300, 400 or 500 bp downstream (or 3′) to exon 45 of the pre-mRNA of the DMD gene.

In some instances, the oligonucleotide (e.g., PMO) molecule hybridizes to a target region that is within exon 45 of the pre-mRNA of the DMD gene. In some instances, the oligonucleotide (e.g., PMO) molecule hybridizes to a target region that is at either the 5′ intron-exon 45 junction or the 3′ exon 45-intron junction of the pre-mRNA of the DMD gene.

In some aspects, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-119. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is from about 24 to about 29 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is from about 24 to about 28 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is from about 24 to about 27 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is from about 24 to about 26 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is from about 24 to about 25 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is from about 25 to about 29 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is from about 25 to about 28 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is from about 25 to about 27 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is from about 25 to about 26 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is from about 26 to about 29 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is from about 26 to about 28 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is from about 26 to about 27 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is from about 27 to about 29 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is from about 27 to about 28 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is from about 28 to about 29 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is at least 25 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is at least 26 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is at least 27 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is at least 28 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is at least 29 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is 20 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is 21 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is 22 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is 23 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is 24 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is 25 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is 26 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is 27 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is 28 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is 29 nucleotides in length. In some instances, the oligonucleotide (e.g., PMO) molecule comprises a sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is 30 nucleotides in length.

In some aspects, the oligonucleotide (e.g., PMO) molecule comprises a core sequence having at least 70%, 75%, 80%, 85%, 90%, 95%, 96%, 97%, 98%, 99%, or 100% sequence identity to a sequence selected from SEQ ID NOs: 100-119. In some aspects, the oligonucleotide (e.g., PMO) molecule comprises a core sequence having at least 70%, 75%, 80%, 85%, 90%, 95%, 96%, 97%, 98%, 99%, or 100% sequence identity to a core sequence of one of SEQ ID NOs: 100-119. In some aspects, the oligonucleotide (e.g., PMO) molecule comprises a core sequence having at least 70%, 75%, 80%, 85%, 90%, 95%, 96%, 97%, 98%, 99%, or 100% sequence identity to a sequence selected from SEQ ID NOs: 100-109. In some aspects, the oligonucleotide (e.g., PMO) molecule comprises a core sequence having at least 70%, 75%, 80%, 85%, 90%, 95%, 96%, 97%, 98%, 99%, or 100% sequence identity to a core sequence of one of SEQ ID NOs: 100-109. In some aspects, the oligonucleotide (e.g., PMO) molecule comprises a core sequence having at least 70%, 75%, 80%, 85%, 90%, 95%, 96%, 97%, 98%, 99%, or 100% sequence identity to a sequence selected from SEQ ID NOs: 100-109, and the oligonucleotide (e.g., PMO) molecule is from about 24 to about 29 nucleotides, at least about 24 to about 28 nucleotides, at least about 24 to about 27 nucleotides, at least about 24 to about 26 nucleotides, at least about 24 to about 25 nucleotides, at least about 25 to about 29 nucleotides, at least about 25 to about 28 nucleotides, at least about 25 to about 27 nucleotides, at least about 25 to about 26 nucleotides, at least about 26 to about 29 nucleotides, at least about 26 to about 28 nucleotides, at least about 26 to about 27 nucleotides, at least about 27 to about 29 nucleotides, at least about 27 to about 28 nucleotides, at least about 28 to about 29 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, or at least 29 nucleotides in length. In some aspects, the oligonucleotide (e.g., PMO) molecule comprises a core sequence having at least 70%, 75%, 80%, 85%, 90%, 95%, 96%, 97%, 98%, 99%, or 100% sequence identity to a core sequence of one of SEQ ID NOs: 100-109, and the oligonucleotide (e.g., PMO) molecule is from about 24 to about 29 nucleotides, at least about 24 to about 28 nucleotides, at least about 24 to about 27 nucleotides, at least about 24 to about 26 nucleotides, at least about 24 to about 25 nucleotides, at least about 25 to about 29 nucleotides, at least about 25 to about 28 nucleotides, at least about 25 to about 27 nucleotides, at least about 25 to about 26 nucleotides, at least about 26 to about 29 nucleotides, at least about 26 to about 28 nucleotides, at least about 26 to about 27 nucleotides, at least about 27 to about 29 nucleotides, at least about 27 to about 28 nucleotides, at least about 28 to about 29 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, or at least 29 nucleotides in length. In some aspects the core sequence refers the nucleic acid sequence of positions 10-20 from the 5′ end, 11-21 from the 5′-end, 12-22 from the 5′ end, 9-21 from the 5′ end, 8-22 from the 5′ end, 7-23 from the 5′ end. In some aspects, the core sequence refers the nucleic acid sequence that is critical in hybridizing the target sequence.

In some aspects, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109. In some cases, the oligonucleotide (e.g., PMO) molecule further comprises 0, 1, 2, 3, or 4 mismatches or no more than 1, 2, 3, or 4 mismatches from a nucleic acid sequence selected from SEQ ID NOs: 100-109. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is from about 24 to about 29 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is from about 24 to about 28 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is from about 24 to about 27 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is from about 24 to about 26 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is from about 24 to about 25 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is from about 25 to about 29 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is from about 25 to about 28 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is from about 25 to about 27 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is from about 25 to about 26 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is from about 26 to about 29 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is from about 26 to about 28 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is from about 26 to about 27 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is from about 27 to about 29 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is from about 27 to about 28 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is from about 28 to about 29 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is at least 25 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is at least 26 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is at least 27 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is at least 28 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is at least 29 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is 20 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is 21 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is 22 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is 23 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is 24 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is 25 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is 26 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is 27 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is 28 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is 29 nucleotides in length. In some instances, the oligonucleotide (e.g., PMO of the PMO-antibody conjugate) comprises at least 10, 15, 16, 17, 18, 19, 20, 21, 22, 23, 24, 25, 26, 27, 28, 29, 30 contiguous bases of a nucleic acid sequence selected from SEQ ID NOs: 100-109 with 0, 1, 2, 3, or 4 mismatches or no more than 1, 2, 3, or 4 mismatches, and the oligonucleotide (e.g., PMO) molecule is 30 nucleotides in length. In some instances, the oligonucleotide (e.g., PMO) molecule consists of a sequence from one of SEQ ID NOs: 116-119.

In some aspects, the oligonucleotide (e.g., PMO of the PMO-antibody conjugate) comprises at least 10, 15, 16, 17, 18, 19, 20 or more contiguous bases of a nucleic acid sequence selected from SEQ ID NOs: 110-119. In some cases, the oligonucleotide (e.g., PMO) molecule further comprises 1, 2, 3, or 4 mismatches or no more than 1, 2, 3, or 4 mismatches from a nucleic acid sequence selected from SEQ ID NOs: 110-119.

In some aspects, the oligonucleotide (e.g., PMO of the PMO-antibody conjugate) comprises a sequence that binds to an acceptor site of exon 45. In some aspects, the oligonucleotide (e.g., PMO) sequence of the oligonucleotide (e.g., PMO)-binding moiety (e.g., antibody) conjugate comprises an oligonucleotide (e.g., PMO) sequence that binds to the acceptor site of SEQ ID NO:200.

In some aspects, the oligonucleotide (e.g., PMO of the PMO-antibody conjugate) comprises an oligonucleotide (e.g., PMO) sequence selected from the group consisting of SEQ ID NOs: 100-109. In some aspects, oligonucleotide (e.g., PMO of the PMO-antibody conjugate) comprises an oligonucleotide (e.g., PMO) sequence selected from the group consisting of SEQ ID NOs: 100-109, and oligonucleotide (e.g., PMO) molecule is from about 24 to about 29 nucleotides, at least about 24 to about 28 nucleotides, at least about 24 to about 27 nucleotides, at least about 24 to about 26 nucleotides, at least about 24 to about 25 nucleotides, at least about 25 to about 29 nucleotides, at least about 25 to about 28 nucleotides, at least about 25 to about 27 nucleotides, at least about 25 to about 26 nucleotides, at least about 26 to about 29 nucleotides, at least about 26 to about 28 nucleotides, at least about 26 to about 27 nucleotides, at least about 27 to about 29 nucleotides, at least about 27 to about 28 nucleotides, at least about 28 to about 29 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, or at least 29 nucleotides in length. In some aspects, the oligonucleotide (e.g., PMO of the PMO-antibody conjugate) comprises an oligonucleotide (e.g., PMO) sequence selected from the group consisting of SEQ ID NOs: 110-119.

In some aspects, the oligonucleotide (e.g., PMO of the PMO-antibody conjugate) comprises the oligonucleotide (e.g., PMO) sequence of SEQ ID NO: 110. In some aspects, the oligonucleotide (e.g., PMO) molecule of the oligonucleotide (e.g., PMO of the PMO-antibody conjugate) comprises the oligonucleotide (e.g., PMO) sequence of SEQ ID NO: 111. In some aspects, the oligonucleotide (e.g., PMO) molecule of the oligonucleotide (e.g., PMO of the PMO-antibody conjugate) comprises the oligonucleotide (e.g., PMO) sequence of SEQ ID NO: 112. In some aspects, the oligonucleotide (e.g., PMO of the PMO-antibody conjugate) comprises the oligonucleotide (e.g., PMO) sequence of SEQ ID NO: 113. In some aspects, the oligonucleotide (e.g., PMO of the PMO-antibody conjugate) comprises the oligonucleotide (e.g., PMO) sequence of SEQ ID NO: 114. In some aspects, the oligonucleotide (e.g., PMO of the PMO-antibody conjugate) comprises the oligonucleotide (e.g., PMO) sequence of SEQ ID NO: 115. In some aspects, the oligonucleotide (e.g., PMO of the PMO-antibody conjugate) comprises the oligonucleotide (e.g., PMO) sequence of SEQ ID NO: 116. In some aspects, the oligonucleotide (e.g., PMO of the PMO-antibody conjugate) comprises the oligonucleotide (e.g., PMO) sequence of SEQ ID NO: 117. In some aspects, the oligonucleotide (e.g., PMO of the PMO-antibody conjugate) comprises the oligonucleotide (e.g., PMO) sequence of SEQ ID NO: 118. In some aspects, the oligonucleotide (e.g., PMO of the PMO-antibody conjugate) comprises the oligonucleotide (e.g., PMO) sequence of SEQ ID NO: 119.

Tables 1 and 2 list the PMO molecules of SEQ ID NOs:100-119.

TABLE 1

| | | SEQ |
|---|---|---|
| PMO | | ID |
| (30-mer) | Sequence (5′-3′) | NO: |
| hEx45_Ac1 | TTTGCCGCTGCCCAATGCCATCCTGGAGTT | 100 |
| hEx45_Ac2 | GTTTGCCGCTGCCCAATGCCATCCTGGAGT | 101 |
| hEx45_Ac3 | AGTTTGCCGCTGCCCAATGCCATCCTGGAG | 102 |
| hEx45_Ac4 | CAGTTTGCCGCTGCCCAATGCCATCCTGGA | 103 |
| hEx45_Ac7 | CAACAGTTTGCCGCTGCCCAATGCCATCCT | 104 |
| hEx45_Ac8 | ACAACAGTTTGCCGCTGCCCAATGCCATCC | 105 |
| hEx45_Ac9 | GACAACAGTTTGCCGCTGCCCAATGCCATC | 106 |
| hEx45_Ac10 | TGACAACAGTTTGCCGCTGCCCAATGCCAT | 107 |
| hEx45_Ac11 | CTGACAACAGTTTGCCGCTGCCCAATGCCA | 108 |
| hEx45_Ac12 | TCTGACAACAGTTTGCCGCTGCCCAATGCC | 109 |

TABLE 2

| | Target | PMO | | SEQ |
|---|---|---|---|---|
| PMO Name | Site | length | PMO Sequence (5′-3′) | ID NO: |
| hEx_45_Ac9_20 | Ac9 | 20 | TGCCGCTGCCCAATGCCATC | 110 |
| hEx_45_Ac9_21 | Ac9 | 21 | TTGCCGCTGCCCAATGCCATC | 111 |
| hEx_45_Ac9_22 | Ac9 | 22 | TTTGCCGCTGCCCAATGCCATC | 112 |
| hEx_45_Ac9_23 | Ac9 | 23 | GTTTGCCGCTGCCCAATGCCATC | 113 |
| hEx_45_Ac9_24 | Ac9 | 24 | AGTTTGCCGCTGCCCAATGCCATC | 114 |
| hEx_45_Ac9_25 | Ac9 | 25 | CAGTTTGCCGCTGCCCAATGCCATC | 115 |
| hEx_45_Ac9_26 | Ac9 | 26 | ACAGTTTGCCGCTGCCCAATGCCATC | 116 |
| hEx_45_Ac9_27 | Ac9 | 27 | AACAGTTTGCCGCTGCCCAATGCCATC | 117 |
| hEx_45_Ac9_28 | Ac9 | 28 | CAACAGTTTGCCGCTGCCCAATGCCATC | 118 |
| hEx_45_Ac9_29 | Ac9 | 29 | ACAACAGTTTGCCGCTGCCCAATGCCATC | 119 |
| hEx_45_Ac9_30 | Ac9 | 30 | GACAACAGTTTGCCGCTGCCCAATGCCATC | 106 |

In some aspects, the polynucleic acid molecule is an antisense oligonucleotide (ASO) or phosphorodiamidate morpholino oligonucleotide (PMO) molecule.

In some aspects, the oligonucleotide (e.g., PMO) molecule is from about 10 to about 50 nucleotides in length. In some instances, the oligonucleotide (e.g., PMO) molecule is from about 10 to about 30, from about 15 to about 30, from about 18 to about 30, from about 18 to about 25, from about 18 to about 24, from about 19 to about 23, from about 19 to about 30, from about 19 to about 25, from about 19 to about 24, from about 19 to about 23, from about 20 to about 30, from about 20 to about 25, from about 20 to about 24, from about 20 to about 23, or from about 20 to about 22 nucleotides in length. In some instances, the oligonucleotide (e.g., PMO) molecule is from about 24 to about 29 nucleotides in length. In some instances, the oligonucleotide (e.g., PMO) molecule is from about 24 to about 28 nucleotides in length. In some instances, the oligonucleotide (e.g., PMO) molecule is from about 24 to about 27 nucleotides in length. In some instances, the oligonucleotide (e.g., PMO) molecule is from about 24 to about 26 nucleotides in length. In some instances, the oligonucleotide (e.g., PMO) molecule is from about 24 to about 25 nucleotides in length. In some instances, the oligonucleotide (e.g., PMO) molecule is from about 25 to about 29 nucleotides in length. In some instances, the oligonucleotide (e.g., PMO) molecule is from about 25 to about 28 nucleotides in length. In some instances, the oligonucleotide (e.g., PMO) molecule is from about 25 to about 27 nucleotides in length. In some instances, the oligonucleotide (e.g., PMO) molecule is from about 25 to about 26 nucleotides in length. In some instances, the oligonucleotide (e.g., PMO) molecule is from about 26 to about 29 nucleotides in length. In some instances, the oligonucleotide (e.g., PMO) molecule is from about 26 to about 28 nucleotides in length. In some instances, the oligonucleotide (e.g., PMO) molecule is from about 26 to about 27 nucleotides in length. In some instances, the oligonucleotide (e.g., PMO) molecule is from about 27 to about 29 nucleotides in length. In some instances, the oligonucleotide (e.g., PMO) molecule is from about 27 to about 28 nucleotides in length. In some instances, the oligonucleotide (e.g., PMO) molecule is from about 28 to about 29 nucleotides in length. In some instances, the oligonucleotide (e.g., PMO) molecule is at least 25 nucleotides in length. In some instances, the oligonucleotide (e.g., PMO) molecule is at least 26 nucleotides in length. In some instances, the oligonucleotide (e.g., PMO) molecule is at least 27 nucleotides in length. In some instances, the oligonucleotide (e.g., PMO) molecule is at least 28 nucleotides in length. In some instances, the oligonucleotide (e.g., PMO) molecule is at least 29 nucleotides in length. In some instances, the oligonucleotide (e.g., PMO) molecule is 20 nucleotides in length. In some instances, the oligonucleotide (e.g., PMO) molecule is 21 nucleotides in length. In some instances, the oligonucleotide (e.g., PMO) molecule is 22 nucleotides in length. In some instances, the oligonucleotide (e.g., PMO) molecule is 23 nucleotides in length. In some instances, the oligonucleotide (e.g., PMO) molecule is 24 nucleotides in length. In some instances, the oligonucleotide (e.g., PMO) molecule is 25 nucleotides in length. In some instances, the oligonucleotide (e.g., PMO) molecule is 26 nucleotides in length. In some instances, the oligonucleotide (e.g., PMO) molecule is 27 nucleotides in length. In some instances, the oligonucleotide (e.g., PMO) molecule is 28 nucleotides in length. In some instances, the oligonucleotide (e.g., PMO) molecule is 29 nucleotides in length. In some instances, the oligonucleotide (e.g., PMO) molecule is 30 nucleotides in length.

In some aspects, the polynucleic acid molecule comprises natural, synthetic, or artificial nucleotide analogues or bases. In some cases, the ASO molecule or the PMO molecule (e.g., PMO of the PMO-antibody conjugate) comprises combinations of DNA, RNA and/or nucleotide analogues. In some instances, the synthetic or artificial nucleotide analogues or bases comprise modifications at one or more of ribose moiety, phosphate moiety, nucleoside moiety, or a combination thereof.

In some aspects, the nucleotide analogues or artificial nucleotide bases comprise a nucleic acid with a modification at a 2′ hydroxyl group of the ribose moiety. In some instances, the modification includes an H, OR, R, halo, SH, SR, NH₂, NHR, NR₂, or CN, wherein R is an alkyl moiety. Exemplary alkyl moieties include, but are not limited to, halogens, sulfurs, thiols, thioethers, thioesters, amines (primary, secondary, or tertiary), amides, ethers, esters, alcohols, and oxygen. In some instances, the alkyl moiety further comprises a modification. In some instances, the modification comprises an azo group, a keto group, an aldehyde group, a carboxyl group, a nitro group, a nitroso group, a nitrile group, a heterocycle (e.g., imidazole, hydrazino or hydroxylamino) group, an isocyanate or cyanate group, or a sulfur containing group (e.g., sulfoxide, sulfone, sulfide, or disulfide). In some instances, the alkyl moiety further comprises a hetero substitution. In some instances, the carbon of the heterocyclic group is substituted by a nitrogen, oxygen or sulfur. In some instances, the heterocyclic substitution includes but is not limited to, morpholino, imidazole, and pyrrolidino.

In some instances, the modification at the 2′ hydroxyl group is a 2′-O-methyl modification or a 2′-O-methoxyethyl (2′-O-MOE) modification. In some cases, the 2′-O-methyl modification adds a methyl group to the 2′ hydroxyl group of the ribose moiety whereas the 2′O-methoxyethyl modification adds a methoxyethyl group to the 2′ hydroxyl group of the ribose moiety. Exemplary chemical structures of a 2′-O-methyl modification of an adenosine molecule and 2′O-methoxyethyl modification of a uridine are illustrated below.

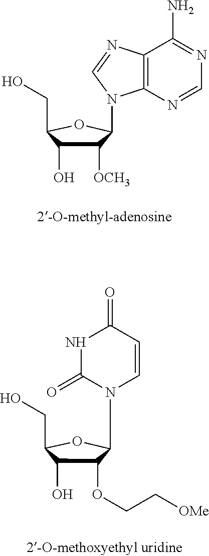

In some instances, the modification at the 2′ hydroxyl group is a 2′-O-aminopropyl modification in which an extended amine group comprising a propyl linker binds the amine group to the 2′ oxygen. In some instances, this modification neutralizes the phosphate derived overall negative charge of the oligonucleotide molecule by introducing one positive charge from the amine group per sugar and thereby improves cellular uptake properties due to its zwitterionic properties. An exemplary chemical structure of a 2′-O-aminopropyl nucleoside phosphoramidite is illustrated below.

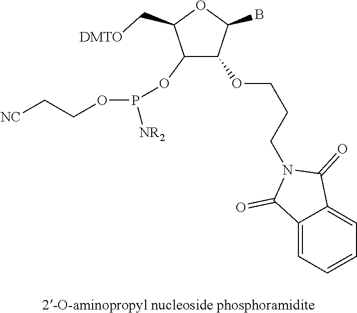

In some instances, the modification at the 2′ hydroxyl group is a locked or bridged ribose modification (e.g., locked nucleic acid or LNA) in which the oxygen molecule bound at the 2′ carbon is linked to the 4′ carbon by a methylene group, thus forming a 2′-C,4′-C-oxy-methylene-linked bicyclic ribonucleotide monomer. Exemplary representations of the chemical structure of LNA are illustrated below. The representation shown to the left highlights the chemical connectivities of an LNA monomer. The representation shown to the right highlights the locked 3′-endo (3E) conformation of the furanose ring of an LNA monomer.

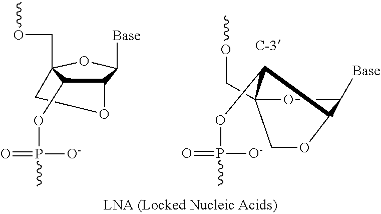

In some instances, the modification at the 2′ hydroxyl group comprises ethylene nucleic acids (ENA) such as for example 2′-4′-ethylene-bridged nucleic acid, which locks the sugar conformation into a C3′-endo sugar puckering conformation. ENAs are part of the bridged nucleic acids class of modified nucleic acids that also comprises LNA. Exemplary chemical structures of the ENA and bridged nucleic acids are illustrated below.

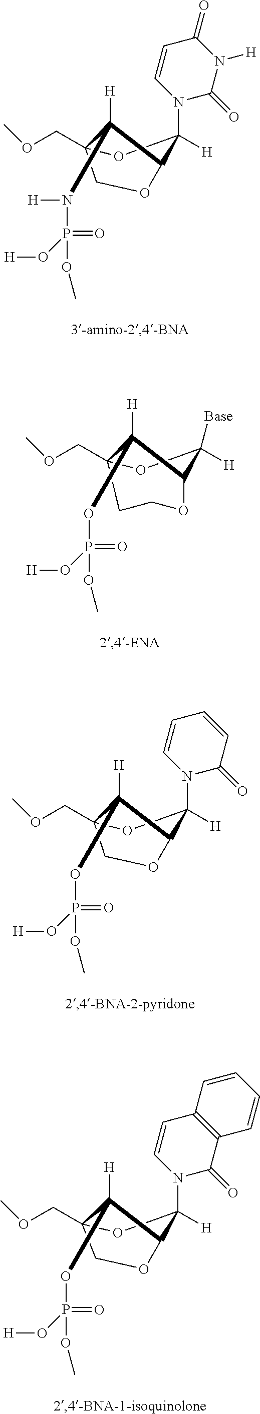

In some aspects, additional modifications at the 2′ hydroxyl group include 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA).

In some aspects, nucleotide analogues comprise modified bases such as, but not limited to, 5-propynyluridine, 5-propynylcytidine, 6-methyladenine, 6-methylguanine, N, N, -dimethyladenine, 2-propyladenine, 2propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2, 2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4, 6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyi nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties, in some cases are or are based on, mannoses, arabinoses, glucopyranoses, galactopyranoses, 4′-thioribose, and other sugars, heterocycles, or carbocycles. The term nucleotide also includes what are known in the art as universal bases. By way of example, universal bases include but are not limited to 3-nitropyrrole, 5-nitroindole, or nebularine.

In some aspects, nucleotide analogues further comprise morpholinos, peptide nucleic acids (PNAs), methylphosphonate nucleotides, thiolphosphonate nucleotides, 2′-fluoro N3-P5′-phosphoramidites, 1′,5′-anhydrohexitol nucleic acids (HNAs), or a combination thereof. Morpholinos or phosphorodiamidate morpholino oligomers (PMOs) comprise synthetic molecules whose structures mimic natural nucleic acid structures by deviating from the normal sugar and phosphate structures. In some instances, the five-member ribose ring is substituted with a six member morpholino ring containing four carbons, one nitrogen and one oxygen. In some cases, the ribose monomers are linked by a phosphordiamidate group instead of a phosphate group. In such cases, the backbone alterations remove all positive and negative charges making morpholinos neutral molecules capable of crossing cellular membranes without the aid of cellular delivery agents such as those used by charged oligonucleotides.

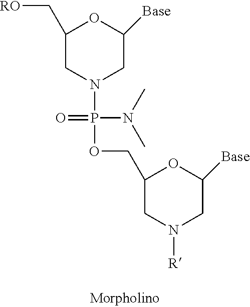

In some aspects, the peptide nucleic acid (PNA) does not contain a sugar ring or phosphate linkage and the bases are attached and appropriately spaced by oligoglycine-like molecules, therefore, eliminating a backbone charge.

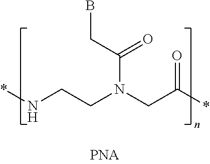

In some aspects, one or more modifications optionally occur at the internucleotide linkage. In some instances, modified internucleotide linkages include, but are not limited to, phosphorothioates, phosphorodithioates, methylphosphonates, 5′-alkylenephosphonates, 5′-methylphosphonates, 3-alkylene phosphonates, borontrifluoridates, borano phosphate esters and selenophosphates with 3-5′ linkages or 2′-5′ linkages, phosphotriesters, thionoalkylphosphotriesters, hydrogen phosphonate linkages, alkyl phosphonates, alkylphosphonothioates, arylphosphonothioates, phosphoroselenoates, phosphorodiselenoates, phosphinates, phosphoramidates, 3′-alkylphosphoramidates, aminoalkylphosphoramidates, thionophosphoramidates, phosphoropiperazidates, phosphoroanilothioates, phosphoroanilidates, ketones, sulfones, sulfonamides, carbonates, carbamates, methylenehydrazos, methylenedimethylhydrazos, formacetals, thioformacetals, oximes, methyleneiminos, methylenemethyliminos, thioamidates, linkages with riboacetyl groups, aminoethyl glycine, silyl or siloxane linkages, alkyl or cycloalkyl linkages with or without heteroatoms of, for example, 1 to 10 carbons that are saturated or unsaturated and/or substituted and/or contain heteroatoms, linkages with morpholino structures, amides, polyamides wherein the bases are attached to the aza nitrogens of the backbone directly or indirectly, and combinations thereof. Phosphorothioate antisense oligonucleotides (PS ASO) are antisense oligonucleotides comprising a phosphorothioate linkage. An exemplary PS ASO is illustrated below.

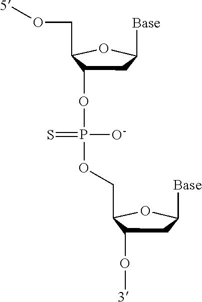

In some instances, the modification is a methyl or thiol modification such as methylphosphonate or thiolphosphonate modification. An exemplary thiolphosphonate nucleotide (left) and an methylphosphonate nucleotide (right) are illustrated below.

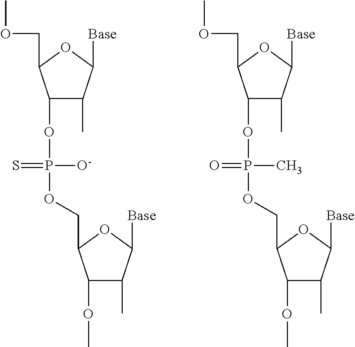

In some instances, a modified nucleotide includes, but is not limited to, 2′-fluoro N3-P5′-phosphoramidites illustrated as:

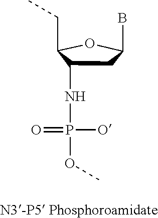

In some instances, a modified nucleotide includes, but is not limited to, hexitol nucleic acid (or 1′,5′-anhydrohexitol nucleic acids (HNA)) illustrated as:

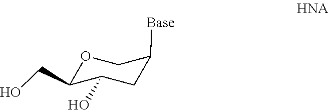

In some aspects, a nucleotide analogue or artificial nucleotide base described above comprises a 5′-vinylphosphonate modified nucleotide with a modification at a 5′ hydroxyl group of the ribose moiety. In some aspects, the 5′-vinylphosphonate modified nucleotide is selected from the nucleotides provided below, wherein X is O or S; and B is a heterocyclic base moiety.

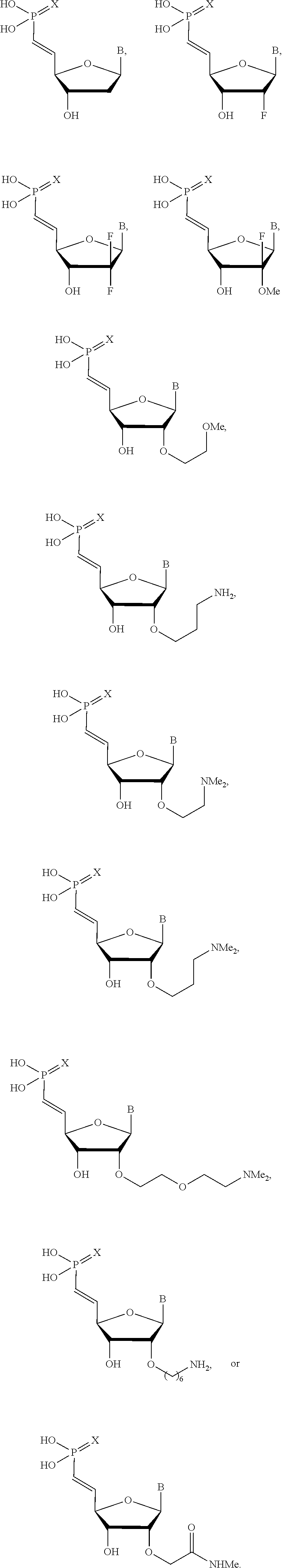

In some instances, the modification at the 2′ hydroxyl group is a 2′-O-aminopropyl modification in which an extended amine group comprising a propyl linker binds the amine group to the 2′ oxygen. In some instances, this modification neutralizes the phosphate-derived overall negative charge of the oligonucleotide molecule by introducing one positive charge from the amine group per sugar and thereby improves cellular uptake properties due to its zwitterionic properties.

In some instances, the 5′-vinylphosphonate modified nucleotide is further modified at the 2′ hydroxyl group in a locked or bridged ribose modification (e.g., locked nucleic acid or LNA) in which the oxygen molecule bound at the 2′ carbon is linked to the 4′ carbon by a methylene group, thus forming a 2′-C, 4′-C-oxy-methylene-linked bicyclic ribonucleotide monomer. Exemplary representations of the chemical structure of 5′-vinylphosphonate modified LNA are illustrated below, wherein X is O or S; B is a heterocyclic base moiety; and J is an internucleotide linking group linking to the adjacent nucleotide of the polynucleotide.

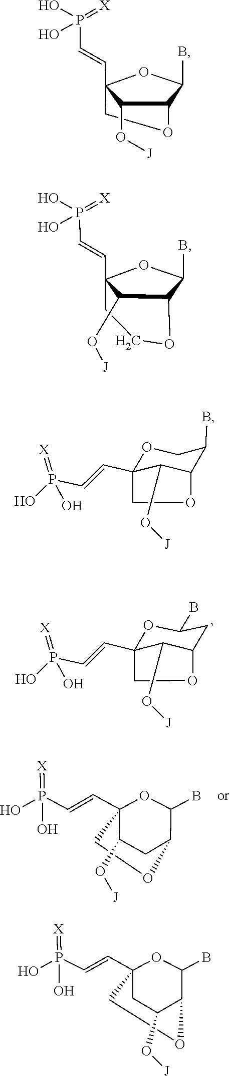

### LNA (Locked Nucleic Acids)

In some aspects, additional modifications at the 2′ hydroxyl group include 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA).

In some aspects, a nucleotide analogue comprises a modified base such as, but not limited to, 5-propynyluridine, 5-propynylcytidine, 6-methyladenine, 6-methylguanine, N, N, -dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2, 2-dimethylguanosine, 5-methylaminoethyluridine, 5-methyloxyuridine, deazanucleotides (such as 7-deaza-adenosine, 6-azouridine, 6-azocytidine, or 6-azothymidine), 5-methyl-2-thiouridine, other thio bases (such as 2-thiouridine, 4-thiouridine, and 2-thiocytidine), dihydrouridine, pseudouridine, queuosine, archaeosine, naphthyl and substituted naphthyl groups, any O- and N-alkylated purines and pyrimidines (such as N6-methyladenosine, 5-methylcarbonylmethyluridine, uridine 5-oxyacetic acid, pyridine-4-one, or pyridine-2-one), phenyl and modified phenyl groups such as aminophenol or 2,4, 6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyi nucleotides, and alkylcarbonylalkylated nucleotides. 5′-Vinylphosphonate modified nucleotides may also include those nucleotides that are modified with respect to the sugar moiety, as well as 5′-vinylphosphonate modified nucleotides having sugars or analogs thereof that are not ribosyl. For example, the sugar moieties, in some cases are or are based on, mannoses, arabinoses, glucopyranoses, galactopyranoses, 4′-thioribose, and other sugars, heterocycles, or carbocycles. The term nucleotide also includes what are known in the art as universal bases. By way of example, universal bases include but are not limited to 3-nitropyrrole, 5-nitroindole, or nebularine.

In some aspects, a 5′-vinylphosphonate modified nucleotide analogue further comprises a morpholino, a peptide nucleic acid (PNA), a methylphosphonate nucleotide, a thiolphosphonate nucleotide, a 2′-fluoro N3-P5′-phosphoramidite, or a 1′,5′-anhydrohexitol nucleic acid (HNA). Morpholinos or phosphorodiamidate morpholino oligomers (PMOs) comprise synthetic molecules whose structures mimic natural nucleic acid structure but deviate from the normal sugar and phosphate structures. In some instances, the five member ribose ring is substituted with a six member morpholino ring containing four carbons, one nitrogen, and one oxygen. In some cases, the ribose monomers are linked by a phosphordiamidate group instead of a phosphate group. In such cases, the backbone alterations remove all positive and negative charges making morpholinos neutral molecules capable of crossing cellular membranes without the aid of cellular delivery agents such as those used by charged oligonucleotides. A non-limiting example of a 5′-vinylphosphonate modified morpholino oligonucleotide is illustrated below, wherein B is a heterocyclic base moiety.

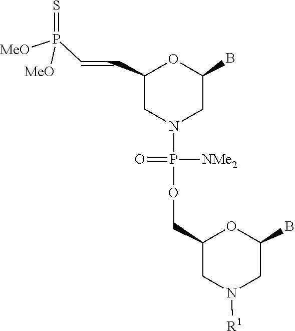

In some aspects, a 5′-vinylphosphonate modified morpholino or PMO described above is a PMO comprising a positive or cationic charge. In some instances, the PMO is PMOplus (Sarepta). PMOplus refers to phosphorodiamidate morpholino oligomers comprising any number of (1-piperazino)phosphinylideneoxy, (1-(4-(omega-guanidino-alkanoyl))-piperazino)phosphinylideneoxy linkages (e.g., as such those described in PCT Publication No. WO2008/036127. In some cases, the PMO is a PMO described in U.S. Pat. No. 7,943,762.

In some aspects, a morpholino or PMO described above is a PMO-X (Sarepta). In some cases, PMO-X refers to phosphorodiamidate morpholino oligomers comprising at least one linkage or at least one of the disclosed terminal modifications, such as those disclosed in PCT Publication No. WO2011/150408 and U.S. Publication No. 2012/0065169.

In some aspects, a morpholino or PMO described above is a PMO as described in Table 5 of U.S. Publication No. 2014/0296321.

Exemplary representations of the chemical structure of 5′-vinylphosphonate modified nucleic acids are illustrated below, wherein X is 0 or S; B is a heterocyclic base moiety; and J is an internucleotide linkage.

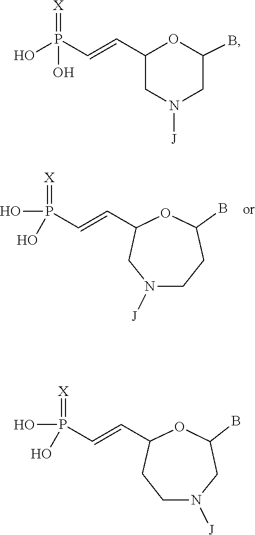

In some aspects, one or more modifications of the 5′-vinylphosphonate modified oligonucleotide optionally occur at the internucleotide linkage. In some instances, modified internucleotide linkages include, but is not limited to, phosphorothioates; phosphorodithioates; methylphosphonates; 5′-alkylenephosphonates; 5′-methylphosphonate; 3′-alkylene phosphonates; borontrifluoridates; borano phosphate esters and selenophosphates with 3′-5′linkages or 2′-5′linkages; phosphotriesters; thionoalkylphosphotriesters; hydrogen phosphonate linkages; alkyl phosphonates; alkylphosphonothioates; arylphosphonothioates; phosphoroselenoates; phosphorodiselenoates; phosphinates; phosphoramidates; 3′-alkylphosphoramidates; aminoalkylphosphoramidates; thionophosphoramidates; phosphoropiperazidates; phosphoroanilothioates; phosphoroanilidates; ketones; sulfones; sulfonamides; carbonates; carbamates; methylenehydrazos; methylenedimethylhydrazos; formacetals; thioformacetals; oximes; methyleneiminos; methylenemethyliminos; thioamidates; linkages with riboacetyl groups; aminoethyl glycine; silyl or siloxane linkages; alkyl or cycloalkyl linkages with or without heteroatoms of, for example, 1 to 10 carbons that are saturated or unsaturated and/or substituted and/or contain heteroatoms; linkages with morpholino structures, amides, or polyamides wherein the bases are attached to the aza nitrogens of the backbone directly or indirectly; and combinations thereof.

In some instances, the modification is a methyl or thiol modification such as methylphosphonate or thiolphosphonate modifications. An exemplary thiolphosphonate nucleotide (left), phosphorodithioates (center) and methylphosphonate nucleotide (right) are illustrated below.

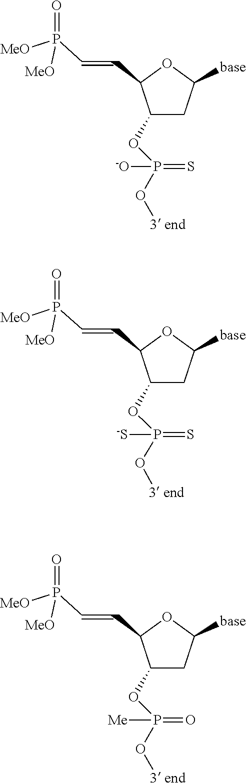

In some instances, a 5′-vinylphosphonate modified nucleotide includes, but is not limited to, phosphoramidites illustrated as:

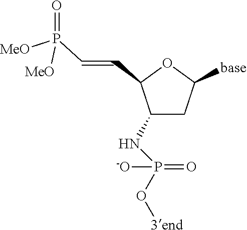

In some instances, the modified internucleotide linkage is a phosphorodiamidate linkage. A non-limiting example of a phosphorodiamidate linkage with a morpholino system is shown below.

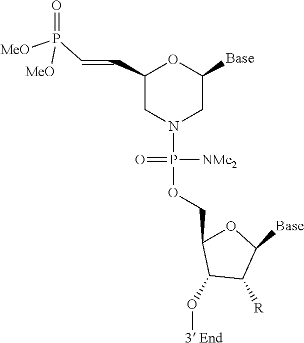

In some instances, the modified internucleotide linkage is a methylphosphonate linkage. A non-limiting example of a methylphosphonate linkage is shown below.

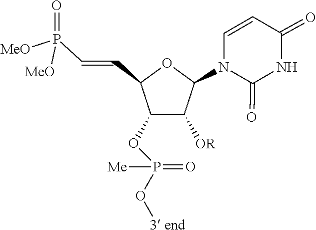

In some instances, the modified internucleotide linkage is an amide linkage. A non-limiting example of an amide linkage is shown below.

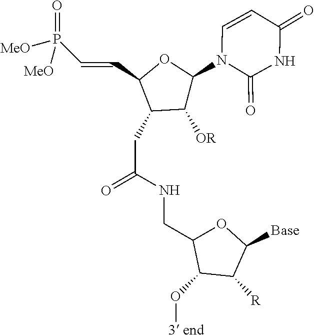

In some instances, a 5′-vinylphosphonate modified nucleotide includes, but is not limited to, the modified nucleic acid illustrated below.

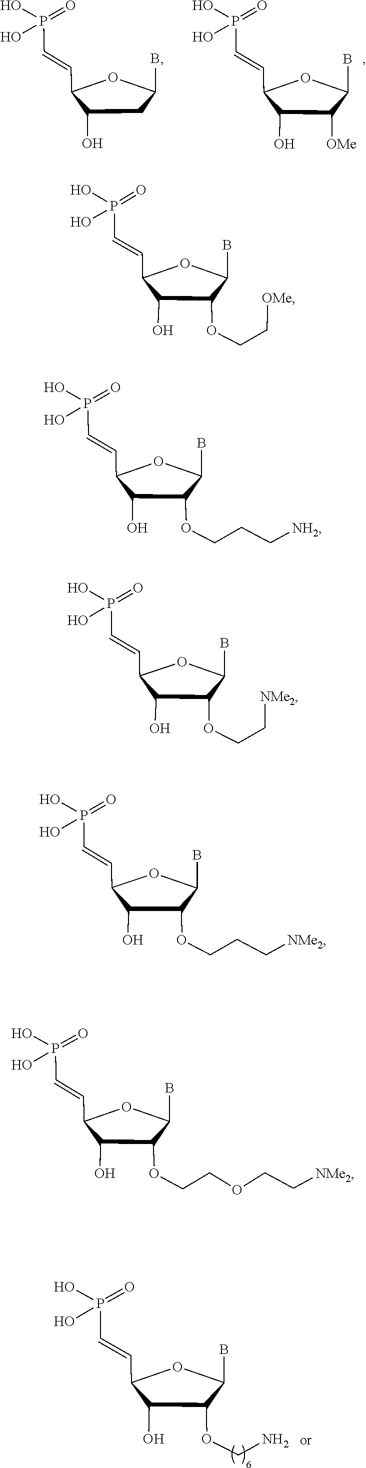

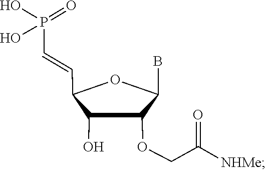

wherein B is a heterocyclic base moiety.

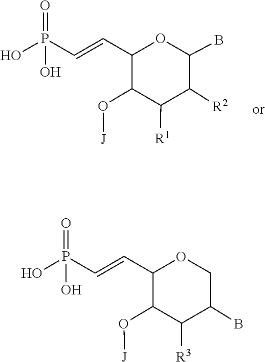

wherein B is a heterocyclic base moiety;
R4, and R5 are independently selected from hydrogen, halogen, alkyl or alkoxy; and
J is an internucleotide linking group linking to the adjacent nucleotide of the polynucleotide.

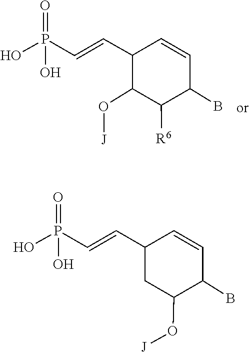

wherein B is a heterocyclic base moiety;
R6 is selected from hydrogen, halogen, alkyl or alkoxy; and
J is an internucleotide linking group linking to the adjacent nucleotide of the polynucleotide.

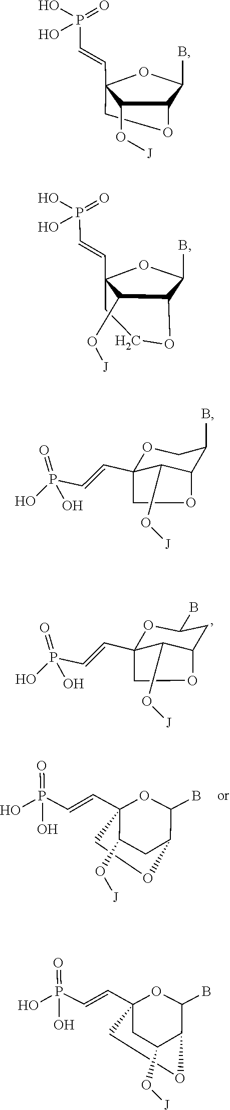

wherein B is a heterocyclic base moiety; and
J is an internucleotide linking group linking to the adjacent nucleotide of the polynucleotide.

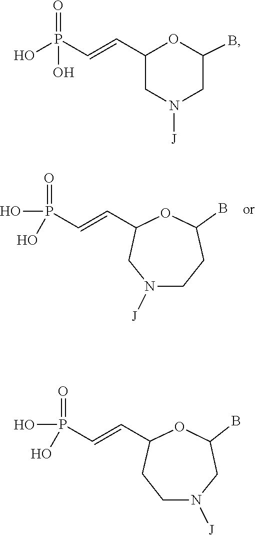

wherein B is a heterocyclic base moiety; and
J is an internucleotide linking group linking to the adjacent nucleotide of the polynucleotide.

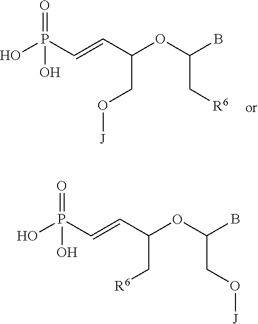

wherein B is a heterocyclic base moiety;
R6 is selected from hydrogen, halogen, alkyl or alkoxy; and
J is an internucleotide linking group linking to the adjacent nucleotide of the polynucleotide.

In some aspects, the oligonucleotide (e.g., PMO of the PMO-antibody conjugate) comprises a plurality of phosphorodiamidate morpholino oligomers or a plurality of peptide nucleic acid-modified non-natural nucleotides, and optionally comprises at least one inverted abasic moiety. In some instances, the oligonucleotide (e.g., PMO) molecule comprises at least 6, 7, 8, 9, 10, 11, 12, 13, 14, 15, 16, 17, 18, 19, 20, or more phosphorodiamidate morpholino oligomer-modified non-natural nucleotides. In some instances, the oligonucleotide (e.g., PMO) molecule comprises 100% phosphorodiamidate morpholino oligomer-modified non-natural nucleotides.

In some instances, the oligonucleotide (e.g., PMO of the PMO-antibody conjugate) comprises at least one of: from about 5% to about 100% modification, from about 10% to about 100% modification, from about 20% to about 100% modification, from about 30% to about 100% modification, from about 40% to about 100% modification, from about 50% to about 100% modification, from about 60% to about 100% modification, from about 70% to about 100% modification, from about 80% to about 100% modification, and from about 90% to about 100% modification.

In some cases, one or more of the artificial nucleotide analogues described herein are resistant toward nucleases such as for example ribonuclease such as RNase H, deoxyribonuclease such as DNase, or exonuclease such as 5′-3′ exonuclease and 3′-5′ exonuclease when compared to natural polynucleic acid molecules. In some instances, artificial nucleotide analogues comprising 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or combinations thereof are resistant toward nucleases such as for example ribonuclease such as RNase H, deoxyribonuclease such as DNase, or exonuclease such as 5′-3′ exonuclease and 3′-5′ exonuclease. In some instances, 2′-O-methyl modified polynucleic acid molecule is nuclease resistant (e.g., RNase H, DNase, 5′-3′ exonuclease or 3′-5′ exonuclease resistance). In some instances, 2′O-methoxyethyl (2′-O-MOE) modified polynucleic acid molecules are nuclease resistant (e.g., RNase H, DNase, 5′-3′ exonuclease or 3′-5′ exonuclease resistance). In some instances, 2′-O-aminopropyl modified polynucleic acid molecules are nuclease resistant (e.g., RNase H, DNase, 5′-3′ exonuclease or 3′-5′ exonuclease resistance). In some instances, 2′-deoxy modified polynucleic acid molecules are nuclease resistant (e.g., RNase H, DNase, 5′-3′ exonuclease or 3′-5′ exonuclease resistance). In some instances, 2′-deoxy-2′-fluoro modified polynucleic acid molecules are nuclease resistant (e.g., RNase H, DNase, 5′-3′ exonuclease or 3′-5′ exonuclease resistance). In some instances, 2′-O-aminopropyl (2′-O-AP) modified polynucleic acid molecules are nuclease resistant (e.g., RNase H, DNase, 5′-3′ exonuclease or 3′-5′ exonuclease resistance). In some instances, 2′-O-dimethylaminoethyl (2′-O-DMAOE) modified polynucleic acid molecules are nuclease resistant (e.g., RNase H, DNase, 5′-3′ exonuclease or 3′-5′ exonuclease resistance). In some instances, 2′-O-dimethylaminopropyl (2′-O-DMAP) modified polynucleic acid molecules are nuclease resistant (e.g., RNase H, DNase, 5′-3′ exonuclease or 3′-5′ exonuclease resistance). In some instances, 2′-O-dimethylaminoethyloxyethyl (2′-O-DMAEOE) modified polynucleic acid molecules are nuclease resistant (e.g., RNase H, DNase, 5′-3′ exonuclease or 3′-5′ exonuclease resistance). In some instances, 2′-O—N-methylacetamido (2′-O-NMA) modified polynucleic acid molecules are nuclease resistant (e.g., RNase H, DNase, 5′-3′ exonuclease or 3′-5′ exonuclease resistance). In some instances, LNA modified polynucleic acid molecules are nuclease resistant (e.g., RNase H, DNase, 5′-3′ exonuclease or 3′-5′ exonuclease resistance). In some instances, ENA modified polynucleic acid molecules are nuclease resistant (e.g., RNase H, DNase, 5′-3′ exonuclease or 3′-5′ exonuclease resistance). In some instances, HNA modified polynucleic acid molecules are nuclease resistant (e.g., RNase H, DNase, 5′-3′ exonuclease or 3′-5′ exonuclease resistance). In some instances, morpholinos are nuclease resistant (e.g., RNase H, DNase, 5′-3′ exonuclease or 3′-5′ exonuclease resistance). In some instances, PNA modified polynucleic acid molecules are resistant to nucleases (e.g., RNase H, DNase, 5′-3′ exonuclease or 3′-5′ exonuclease resistance). In some instances, methylphosphonate nucleotides modified polynucleic acid molecule are nuclease resistant (e.g., RNase H, DNase, 5′-3′ exonuclease or 3′-5′ exonuclease resistance). In some instances, thiolphosphonate nucleotide modified polynucleic acid molecules are nuclease resistant (e.g., RNase H, DNase, 5′-3′ exonuclease or 3′-5′ exonuclease resistance). In some instances, polynucleic acid molecules comprising 2′-fluoro N3-P5′-phosphoramidites are nuclease resistant (e.g., RNase H, DNase, 5′-3′ exonuclease or 3′-5′ exonuclease resistance). In some instances, the 5′ conjugates described herein inhibit 5′-3′ exonucleolytic cleavage. In some instances, the 3′ conjugates described herein inhibit 3′-5′ exonucleolytic cleavage.

### Polynucleic Acid Molecule Synthesis

In some aspects, a polynucleic acid molecule described herein is constructed using chemical synthesis and/or enzymatic ligation reactions using procedures known in the art. For example, a polynucleic acid molecule is chemically synthesized using naturally occurring nucleotides or variously modified nucleotides designed to increase the biological stability of the molecules or to increase the physical stability of the duplex formed between the polynucleic acid molecule and target nucleic acids. Exemplary methods include those described in: U.S. Pat. Nos. 5,142,047; 5,185,444; 5,889,136; 6,008,400; and 6,111,086; PCT Publication No. WO2009099942; or European Publication No. 1579015. Additional exemplary methods include those described in: Griffey et al., “2′-O-aminopropyl ribonucleotides: a zwitterionic modification that enhances the exonuclease resistance and biological activity of antisense oligonucleotides,” J. Med. Chem. 39(26):5100-5109 (1997)); Obika, et al. “Synthesis of 2′-0,4′-C-methyleneuridine and -cytidine. Novel bicyclic nucleosides having a fixed C3, -endo sugar puckering”. Tetrahedron Letters 38 (50): 8735 (1997); Koizumi, M. “ENA oligonucleotides as therapeutics”. Current opinion in molecular therapeutics 8 (2): 144-149 (2006); and Abramova et al., “Novel oligonucleotide analogues based on morpholino nucleoside subunits-antisense technologies: new chemical possibilities,” Indian Journal of Chemistry 48B:1721-1726 (2009). Alternatively, the polynucleic acid molecule is produced biologically using an expression vector into which a polynucleic acid molecule has been subcloned in an antisense orientation (i.e., RNA transcribed from the inserted polynucleic acid molecule will be of an antisense orientation to a target polynucleic acid molecule of interest).

In some aspects, a polynucleic acid molecule is synthesized via a tandem synthesis methodology, wherein both strands are synthesized as a single contiguous oligonucleotide fragment or strand separated by a cleavable linker which is subsequently cleaved to provide separate fragments or strands that hybridize and permit purification of the duplex.

Additional modification methods for incorporating, for example, sugar, base and phosphate modifications include: Eckstein et al., International Patent Publication No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Patent Publication No. WO 93/15187; Sproat, U.S. Pat. No. 5,334,711 and Beigelman et al., 1995, J. Biol. Chem., 270, 25702; Beigelman et al., International Patent Publication No. WO 97/26270; Beigelman et al., U.S. Pat. No. 5,716,824; Usman et al., U.S. Pat. No. 5,627,053; Woolf et al., International Patent Publication No. WO 98/13526; Thompson et al., U.S. Ser. No. 60/082,404 which was filed on Apr. 20, 1998; Karpeisky et al., 1998, Tetrahedron Lett., 39, 1131; Earnshaw and Gait, 1998, Biopolymers (Nucleic Acid Sciences), 48, 39-55; Verma and Eckstein, 1998, Annu. Rev. Biochem., 67, 99-134; and Burlina et al., 1997, Bioorg. Med. Chem., 5, 1999-2010. Such publications describe general methods and strategies to determine the location of incorporation of sugar, base and/or phosphate modifications and the like into nucleic acid molecules without modulating catalysis.

In some instances, while chemical modification of the polynucleic acid molecule internucleotide linkages with phosphorothioate, phosphorodithioate, and/or 5′-methylphosphonate linkages improves stability, excessive modifications sometimes cause toxicity or decreased activity. Therefore, when designing nucleic acid molecules, the amount of these internucleotide linkages in some cases is minimized. In such cases, the reduction in the concentration of these linkages lowers toxicity, increases efficacy and higher specificity of these molecules.

### Binding Moiety/Antibody

In some aspects, provided herein is an oligonucleotide conjugate comprising a binding moiety for delivering to a muscle cell conjugated to an oligonucleotide molecule. In some instances, the oligonucleotide molecule is an ASO or a PMO. In some instances, the binding moiety binds to a cell surface molecule of the muscle cell. In some instances, the binding moiety binds to a transferrin receptor.

In some instances, the binding moiety is a small molecule that that binds to the cell surface molecule of the muscle cell described herein. In some instances, the binding moiety is a small molecule that binds to the cell surface receptor of the muscle cell described herein. In some instances, the binding moiety is polypeptide that that binds to a transferrin receptor. In some instances, the binding moiety is an antibody or antigen binding fragment thereof that binds to the cell surface molecule of the muscle cell described herein. In some instances, the binding moiety is an antibody or antigen binding fragment thereof that binds to the cell surface receptor of the muscle cell described herein. In some instances, the binding moiety is an antibody or antigen binding fragment thereof that binds to a transferrin receptor on the muscle cell.

In some aspects, the antibody or antigen binding fragment thereof comprises a humanized antibody or antigen binding fragment thereof, murine antibody or antigen binding fragment thereof, chimeric antibody or antigen binding fragment thereof, monoclonal antibody or antigen binding fragment thereof, monovalent Fab, Fab′, divalent Fab2, F(ab)′3 fragments, single-chain variable fragment (scFv), bis-scFv, (scFv)2, diabody, minibody, nanobody, triabody, tetrabody, disulfide stabilized Fv protein (dsFv), single-domain antibody (sdAb), Ig NAR, camelid antibody or antigen binding fragment thereof, bispecific antibody or antigen binding fragment thereof, or a chemically modified derivative thereof.

In some instances, the antibody is an anti-transferrin receptor (anti-CD71) antibody or antigen binding fragment thereof. In some cases, the anti-transferrin receptor antibody is a humanized antibody or antigen binding fragment thereof. In other cases, the anti-transferrin receptor antibody is a chimeric antibody or antigen binding fragment thereof. In additional cases, the anti-transferrin receptor antibody is a monovalent, a divalent, or a multi-valent antibody or antigen binding fragment thereof. In some aspects, exemplary anti-transferrin receptor antibodies or antigen binding fragments thereof include MAB5746 from R&D Systems, AHP858 from Bio-Rad Laboratories, A80-128A from Bethyl Laboratories, Inc., and T2027 from MilliporeSigma. In some aspects, the anti-transferrin receptor antibody or antigen binding fragment thereof includes the antibodies disclosed in U.S. Pat. No. 10,913,800 or U.S. Pat. No. 11,028,179.

In some instances, the anti-transferrin receptor antibody comprises a variable heavy chain (VH) region and a variable light chain (VL) region, wherein the VH region comprises an HCDR1 sequence comprising or consisting of a sequence of SEQ ID NO: 17; an HCDR2 sequence comprising or consisting of a sequence of EINPIX1GRSNYAX2KFQG (SEQ ID NO: 12), wherein X1 is selected from N or Q and X2 is selected from Q or E; and an HCDR3 sequence comprising or consisting of a sequence of SEQ ID NO: 19.

In some aspects, the VH region of the anti-transferring antibody comprises HCDR1, HCDR2, and HCDR3 sequences selected from Table 3.

TABLE 3

| | | SEQ ID | | SEQ ID | | SEQ ID |
|---|---|---|---|---|---|---|
| Name | HCDR1 | NO: | HCDR2 | NO: | HCDR3 | NO: |
| 13E4_VH1 | YTFTNYWMH | 17 | EINPINGRSNYAQKFQG | 18 | GTRAMHY | 19 |
| 13E4_VH2* | YTFTNYWMH | 17 | EINPINGRSNYAEKFQG | 20 | GTRAMHY | 19 |
| 13E4_VH3 | YTFTNYWMH | 17 | EINPIQGRSNYAEKFQG | 21 | GTRAMHY | 19 |

*13E4_VH2 shares the same HCDR1, HCDR2, and HCDR3 sequences with anti-transferrin receptor antibody 13E4_VH4

In some aspects, the VH region comprises an HCDR1 sequence comprising or consisting of a sequence of SEQ ID NO: 17; an HCDR2 sequence comprising or consisting of a sequence of SEQ ID NO: 18, 20, or 21; and an HCDR3 sequence comprising or consisting of a sequence of SEQ ID NO: 19. In some instances, the VH region comprises an HCDR1 sequence comprising or consisting of a sequence of SEQ ID NO: 17, an HCDR2 sequence comprising or consisting of a sequence of SEQ ID NO: 18, and an HCDR3 sequence comprising or consisting of a sequence of SEQ ID NO: 19. In some instances, the VH region comprises an HCDR1 sequence comprising or consisting of a sequence of SEQ ID NO: 17, an HCDR2 sequence comprising or consisting of a sequence of SEQ ID NO: 20, and an HCDR3 sequence comprising or consisting of a sequence of SEQ ID NO: 19. In some instances, the VH region comprises an HCDR1 sequence comprising or consisting of a sequence of SEQ ID NO: 17, an HCDR2 sequence comprising or consisting of a sequence of SEQ ID NO: 21, and an HCDR3 sequence comprising or consisting of a sequence of SEQ ID NO: 19.

In some aspects, the VL region of the anti-transferrin receptor antibody comprises an LCDR1 sequence comprising or consisting of a sequence of RTSENIYX3NLA (SEQ ID NO: 13), an LCDR2 sequence comprising or consisting of a sequence of AX4TNLAX5 (SEQ ID NO: 14), and an LCDR3 sequence comprising or consisting of a sequence of QHFWGTPLTX6 (SEQ ID NO: 15), wherein X3 is selected from N or S, X4 is selected from A or G, X5 is selected from D or E, and X6 is present or absent, and if present, is F.

In some aspects, the VL region of the anti-transferrin receptor antibody comprises LCDR1, LCDR2, and LCDR3 sequences selected from Table 4.

TABLE 4

| | | SEQ | | SEQ | | SEQ |
|---|---|---|---|---|---|---|
| | | ID | | ID | | ID |
| Name | LCDR1 | NO: | LCDR2 | NO: | LCDR3 | NO: |
| 13E4_VL1* | RTSENIYNNLA | 22 | AATNLAD | 23 | QHFWGTPLT | 24 |
| 13E4_VL3 | RTSENIYNNLA | 22 | AATNLAE | 25 | QHFWGTPLTF | 26 |
| 13E4_VL4 | RTSENIYSNLA | 27 | AGTNLAD | 28 | QHFWGTPLTF | 26 |

*13E4_VL1 shares the same LCDR1, LCDR2, and LCDR3 sequences with anti-transferrin receptor antibody 13E4_VL2

In some instances, the VL region comprises an LCDR1 sequence comprising or consisting of a sequence of SEQ ID NO: 13, an LCDR2 sequence comprising or consisting of a sequence of SEQ ID NO: 23, 25, or 28, and an LCDR3 sequence comprising or consisting of a sequence of SEQ ID NO: 24 or 26, wherein X3 is selected from N or S.

In some instances, the VL region comprises an LCDR1 sequence comprising or consisting of a sequence of SEQ ID NO: 22 or 27, an LCDR2 sequence comprising or consisting of a sequence of SEQ ID NO: 14, and an LCDR3 sequence comprising or consisting of a sequence of SEQ ID NO: 24 or 26, wherein X4 is selected from A or G, and X5 is selected from D or E.

In some instances, the VL region comprises an LCDR1 sequence comprising or consisting of a sequence of SEQ ID NO: 22 or 27, an LCDR2 sequence comprising or consisting of a sequence of SEQ ID NO: 23, 25, or 28, and an LCDR3 sequence comprising or consisting of a sequence of SEQ ID NO: 15, wherein X6 is present or absent, and if present, is F.

In some instances, the VL region comprises an LCDR1 sequence comprising or consisting of a sequence of SEQ ID NO: 22, an LCDR2 sequence comprising or consisting of a sequence of AATNLAX5 (SEQ ID NO: 16), and an LCDR3 sequence comprising or consisting of a sequence of SEQ ID NO: 15, wherein X5 is selected from D or E and X6 is present or absent, and if present, is F.

In some instances, the VL region comprises an LCDR1 sequence comprising or consisting of a sequence of SEQ ID NO: 22, an LCDR2 sequence comprising or consisting of a sequence of SEQ ID NO: 23, and an LCDR3 sequence comprising or consisting of a sequence of SEQ ID NO: 24.

In some instances, the VL region comprises an LCDR1 sequence comprising or consisting of a sequence of SEQ ID NO: 22, an LCDR2 sequence comprising or consisting of a sequence of SEQ ID NO: 25, and an LCDR3 sequence comprising or consisting of a sequence of SEQ ID NO: 26.

In some instances, the VL region comprises an LCDR1 sequence comprising or consisting of a sequence of SEQ ID NO: 27, an LCDR2 sequence comprising or consisting of a sequence of SEQ ID NO: 28, and an LCDR3 sequence comprising or consisting of a sequence of SEQ ID NO: 26.

In some aspects, the anti-transferrin receptor antibody comprises a VH region and a VL region, wherein the VH region comprises an HCDR1 sequence comprising or consisting of a sequence of SEQ ID NO: 17; an HCDR2 sequence comprising or consisting of a sequence of SEQ ID NO: 12, wherein X1 is selected from N or Q and X2 is selected from Q or E; and an HCDR3 sequence comprising or consisting of a sequence of SEQ ID NO: 19; and the VL region comprises an LCDR1 sequence comprising or consisting of a sequence of SEQ ID NO: 13, an LCDR2 sequence comprising or consisting of a sequence of SEQ ID NO: 14, and an LCDR3 sequence comprising or consisting of a sequence of SEQ ID NO: 15, wherein X3 is selected from N or S, X4 is selected from A or G, X5 is selected from D or E, and X6 is present or absent, and if present, is F.

In some instances, the anti-transferrin receptor antibody comprises a VH region and a VL region, wherein the VH region comprises an HCDR1 sequence comprising or consisting of a sequence of SEQ ID NO: 17; an HCDR2 sequence comprising or consisting of a sequence of SEQ ID NO: 12, wherein X1 is selected from N or Q and X2 is selected from Q or E; and an HCDR3 sequence comprising or consisting of a sequence of SEQ ID NO: 19; and the VL region comprises an LCDR1 sequence comprising or consisting of a sequence of SEQ ID NO: 13, an LCDR2 sequence comprising or consisting of a sequence of SEQ ID NO: 23, 25, or 28, and an LCDR3 sequence comprising or consisting of a sequence of SEQ ID NO: 24 or 26, wherein X3 is selected from N or S.

In some instances, the anti-transferrin receptor antibody comprises a VH region and a VL region, wherein the VH region comprises an HCDR1 sequence comprising or consisting of a sequence of SEQ ID NO: 17; an HCDR2 sequence comprising or consisting of a sequence of SEQ ID NO: 12, wherein X1 is selected from N or Q and X2 is selected from Q or E; and an HCDR3 sequence comprising or consisting of a sequence of SEQ ID NO: 19; and the VL region comprises an LCDR1 sequence comprising or consisting of a sequence of SEQ ID NO: 22 or 27, an LCDR2 sequence comprising or consisting of a sequence of SEQ ID NO: 14, and an LCDR3 sequence comprising or consisting of a sequence of SEQ ID NO: 24 or 26, wherein X4 is selected from A or G, and X5 is selected from D or E.

In some instances, the anti-transferrin receptor antibody comprises a VH region and a VL region, wherein the VH region comprises an HCDR1 sequence comprising or consisting of a sequence of SEQ ID NO: 17; an HCDR2 sequence comprising or consisting of a sequence of SEQ ID NO: 12, wherein X1 is selected from N or Q and X2 is selected from Q or E; and an HCDR3 sequence comprising or consisting of a sequence of SEQ ID NO: 19; and the VL region comprises an LCDR1 sequence comprising or consisting of a sequence of SEQ ID NO: 22 or 27, an LCDR2 sequence comprising or consisting of a sequence of SEQ ID NO: 23, 25, or 28, and an LCDR3 sequence comprising or consisting of a sequence of SEQ ID NO: 15, wherein X6 is present or absent, and if present, is F.

In some instances, the anti-transferrin receptor antibody comprises a VH region and a VL region, wherein the VH region comprises an HCDR1 sequence comprising or consisting of a sequence of SEQ ID NO: 17; an HCDR2 sequence comprising or consisting of a sequence of SEQ ID NO: 12, wherein X1 is selected from N or Q and X2 is selected from Q or E; and an HCDR3 sequence comprising or consisting of a sequence of SEQ ID NO: 19; and the VL region comprises an LCDR1 sequence comprising or consisting of a sequence of SEQ ID NO: 22, an LCDR2 sequence comprising or consisting of a sequence of SEQ ID NO: 16, and an LCDR3 sequence comprising or consisting of a sequence of SEQ ID NO: 15, wherein X5 is selected from D or E and X6 is present or absent, and if present, is F.

In some instances, the anti-transferrin receptor antibody comprises a VH region and a VL region, wherein the VH region comprises an HCDR1 sequence comprising or consisting of a sequence of SEQ ID NO: 17; an HCDR2 sequence comprising or consisting of a sequence of SEQ ID NO: 12, wherein X1 is selected from N or Q and X2 is selected from Q or E; and an HCDR3 sequence comprising or consisting of a sequence of SEQ ID NO: 19; and the VL region comprises an LCDR1 sequence comprising or consisting of a sequence of SEQ ID NO: 22, an LCDR2 sequence comprising or consisting of a sequence of SEQ ID NO: 23, and an LCDR3 sequence comprising or consisting of a sequence of SEQ ID NO: 24.

In some instances, the anti-transferrin receptor antibody comprises a VH region and a VL region, wherein the VH region comprises an HCDR1 sequence comprising or consisting of a sequence of SEQ ID NO: 17; an HCDR2 sequence comprising or consisting of a sequence of SEQ ID NO: 12, wherein X1 is selected from N or Q and X2 is selected from Q or E; and an HCDR3 sequence comprising or consisting of a sequence of SEQ ID NO: 19; and the VL region comprises an LCDR1 sequence comprising or consisting of a sequence of SEQ ID NO: 22, an LCDR2 sequence comprising or consisting of a sequence of SEQ ID NO: 25, and an LCDR3 sequence comprising or consisting of a sequence of SEQ ID NO: 26.

In some instances, the anti-transferrin receptor antibody comprises a VH region and a VL region, wherein the VH region comprises an HCDR1 sequence comprising or consisting of a sequence of SEQ ID NO: 17; an HCDR2 sequence comprising or consisting of a sequence of SEQ ID NO: 12, wherein X1 is selected from N or Q and X2 is selected from Q or E; and an HCDR3 sequence comprising or consisting of a sequence of SEQ ID NO: 19; and the VL region comprises an LCDR1 sequence comprising or consisting of a sequence of SEQ ID NO: 27, an LCDR2 sequence comprising or consisting of a sequence of SEQ ID NO: 28, and an LCDR3 sequence comprising or consisting of a sequence of SEQ ID NO: 26.

In some instances, the anti-transferrin receptor antibody comprises a VH region and a VL region, in which the VH region comprises an HCDR1 sequence comprising or consisting of a sequence of SEQ ID NO: 17, an HCDR2 sequence comprising or consisting of a sequence of SEQ ID NO: 18, and an HCDR3 sequence comprising or consisting of a sequence of SEQ ID NO: 19; and the VL region comprises an LCDR1 sequence comprising or consisting of a sequence of SEQ ID NO: 13, an LCDR2 sequence comprising or consisting of a sequence of SEQ ID NO: 23, 25, or 28, and an LCDR3 sequence comprising or consisting of a sequence of SEQ ID NO: 24 or 26, wherein X3 is selected from N or S.

In some instances, the anti-transferrin receptor antibody comprises a VH region and a VL region, in which the VH region comprises an HCDR1 sequence comprising or consisting of a sequence of SEQ ID NO: 17, an HCDR2 sequence comprising or consisting of a sequence of SEQ ID NO: 18, and an HCDR3 sequence comprising or consisting of a sequence of SEQ ID NO: 19; and the VL region comprises an LCDR1 sequence comprising or consisting of a sequence of SEQ ID NO: 22 or 27, an LCDR2 sequence comprising or consisting of a sequence of SEQ ID NO: 14, and an LCDR3 sequence comprising or consisting of a sequence of SEQ ID NO: 24 or 26, wherein X4 is selected from A or G, and X5 is selected from D or E.

In some instances, the anti-transferrin receptor antibody comprises a VH region and a VL region, in which the VH region comprises an HCDR1 sequence comprising or consisting of a sequence of SEQ ID NO: 17, an HCDR2 sequence comprising or consisting of a sequence of SEQ ID NO: 18, and an HCDR3 sequence comprising or consisting of a sequence of SEQ ID NO: 19; and the VL region comprises an LCDR1 sequence comprising or consisting of a sequence of SEQ ID NO: 22 or 27, an LCDR2 sequence comprising or consisting of a sequence of SEQ ID NO: 23, 25, or 28, and LCDR3 sequence comprising or consisting of a sequence of SEQ ID NO: 15, wherein X6 is present or absent, and if present, is F.

In some instances, the anti-transferrin receptor antibody comprises a VH region and a VL region, in which the VH region comprises an HCDR1 sequence comprising or consisting of a sequence of SEQ ID NO: 17, an HCDR2 sequence comprising or consisting of a sequence of SEQ ID NO: 18, and an HCDR3 sequence comprising or consisting of a sequence of SEQ ID NO: 19; and the VL region comprises an LCDR1 sequence comprising or consisting of a sequence of SEQ ID NO: 22, an LCDR2 sequence comprising or consisting of a sequence of SEQ ID NO: 16, and an LCDR3 sequence comprising or consisting of a sequence of SEQ ID NO: 15, wherein X5 is selected from D or E and X6 is present or absent, and if present, is F.

In some instances, the anti-transferrin receptor antibody comprises a VH region and a VL region, in which the VH region comprises an HCDR1 sequence comprising or consisting of a sequence of SEQ ID NO: 17, an HCDR2 sequence comprising or consisting of a sequence of SEQ ID NO: 18, and an HCDR3 sequence comprising or consisting of a sequence of SEQ ID NO: 19; and the VL region comprises an LCDR1 sequence comprising or consisting of a sequence of SEQ ID NO: 22, an LCDR2 sequence comprising or consisting of a sequence of SEQ ID NO: 23, and an LCDR3 sequence comprising or consisting of a sequence of SEQ ID NO: 24.

In some instances, the anti-transferrin receptor antibody comprises a VH region and a VL region, in which the VH region comprises an HCDR1 sequence comprising or consisting of a sequence of SEQ ID NO: 17, an HCDR2 sequence comprising or consisting of a sequence of SEQ ID NO: 18, and an HCDR3 sequence comprising or consisting of a sequence of SEQ ID NO: 19; and the VL region comprises an LCDR1 sequence comprising or consisting of a sequence of SEQ ID NO: 22, an LCDR2 sequence comprising or consisting of a sequence of SEQ ID NO: 21, and an LCDR3 sequence comprising or consisting of a sequence of SEQ ID NO: 26.

In some instances, the anti-transferrin receptor antibody comprises a VH region and a VL region, in which the VH region comprises an HCDR1 sequence comprising or consisting of a sequence of SEQ ID NO: 17, an HCDR2 sequence comprising or consisting of a sequence of SEQ ID NO: 18, and an HCDR3 sequence comprising or consisting of a sequence of SEQ ID NO: 19; and the VL region comprises an LCDR1 sequence comprising or consisting of a sequence of SEQ ID NO: 27, an LCDR2 sequence comprising or consisting of a sequence of SEQ ID NO: 28, and an LCDR3 sequence comprising or consisting of a sequence of SEQ ID NO: 26.

In some instances, the anti-transferrin receptor antibody comprises a VH region and a VL region, in which the VH region comprises an HCDR1 sequence comprising or consisting of a sequence of SEQ ID NO: 17, an HCDR2 sequence comprising or consisting of a sequence of SEQ ID NO: 20, and an HCDR3 sequence comprising or consisting of a sequence of SEQ ID NO: 19; and the VL region comprises an LCDR1 sequence comprising or consisting of a sequence of SEQ ID NO: 13, an LCDR2 sequence comprising or consisting of a sequence of SEQ ID NO: 23, 25, or 28, and an LCDR3 sequence comprising or consisting of a sequence of SEQ ID NO: 24 or 26, wherein X3 is selected from N or S.

In some instances, the anti-transferrin receptor antibody comprises a VH region and a VL region, in which the VH region comprises an HCDR1 sequence comprising or consisting of a sequence of SEQ ID NO: 17, an HCDR2 sequence comprising or consisting of a sequence of SEQ ID NO: 20, and an HCDR3 sequence comprising or consisting of a sequence of SEQ ID NO: 19; and the VL region comprises an LCDR1 sequence comprising or consisting of a sequence of SEQ ID NO: 22 or 27, an LCDR2 sequence comprising or consisting of a sequence of SEQ ID NO: 14, and an LCDR3 sequence comprising or consisting of a sequence of SEQ ID NO: 24 or 26, wherein X4 is selected from A or G, and X5 is selected from D or E.

In some instances, the anti-transferrin receptor antibody comprises a VH region and a VL region, in which the VH region comprises an HCDR1 sequence comprising or consisting of a sequence of SEQ ID NO: 17, an HCDR2 sequence comprising or consisting of a sequence of SEQ ID NO: 20, and an HCDR3 sequence comprising or consisting of a sequence of SEQ ID NO: 19; and the VL region comprises an LCDR1 sequence comprising or consisting of a sequence of SEQ ID NO: 22 or 27, an LCDR2 sequence comprising or consisting of a sequence of SEQ ID NO: 23, 25 or 28, and an LCDR3 sequence comprising or consisting of a sequence of SEQ ID NO: 15, wherein X6 is present or absent, and if present, is F.

In some instances, the anti-transferrin receptor antibody comprises a VH region and a VL region, in which the VH region comprises an HCDR1 sequence comprising or consisting of a sequence of SEQ ID NO: 17, an HCDR2 sequence comprising or consisting of a sequence of SEQ ID NO: 20, and an HCDR3 sequence comprising or consisting of a sequence of SEQ ID NO: 19; and the VL region comprises an LCDR1 sequence comprising or consisting of a sequence of SEQ ID NO: 22, an LCDR2 sequence comprising or consisting of a sequence of SEQ ID NO: 16, and an LCDR3 sequence comprising or consisting of a sequence of SEQ ID NO: 15, wherein X5 is selected from D or E and X6 is present or absent, and if present, is F.

In some instances, the anti-transferrin receptor antibody comprises a VH region and a VL region, in which the VH region comprises an HCDR1 sequence comprising or consisting of a sequence of SEQ ID NO: 17, an HCDR2 sequence comprising or consisting of a sequence of SEQ ID NO: 20, and an HCDR3 sequence comprising or consisting of a sequence of SEQ ID NO: 19; and the VL region comprises an LCDR1 sequence comprising or consisting of a sequence of SEQ ID NO: 22, an LCDR2 sequence comprising or consisting of a sequence of SEQ ID NO: 23, and an LCDR3 sequence comprising or consisting of a sequence of SEQ ID NO: 24.

In some instances, the anti-transferrin receptor antibody comprises a VH region and a VL region, in which the VH region comprises an HCDR1 sequence comprising or consisting of a sequence of SEQ ID NO: 17, an HCDR2 sequence comprising or consisting of a sequence of SEQ ID NO: 20, and an HCDR3 sequence comprising or consisting of a sequence of SEQ ID NO: 19; and the VL region comprises an LCDR1 sequence comprising or consisting of a sequence of SEQ ID NO: 22, an LCDR2 sequence comprising or consisting of a sequence of SEQ ID NO: 25, and an LCDR3 sequence comprising or consisting of a sequence of SEQ ID NO:26.

In some instances, the anti-transferrin receptor antibody comprises a VH region and a VL region, in which the VH region comprises an HCDR1 sequence comprising or consisting of a sequence of SEQ ID NO: 17, an HCDR2 sequence comprising or consisting of a sequence of SEQ ID NO: 20, and an HCDR3 sequence comprising or consisting of a sequence of SEQ ID NO: 19; and the VL region comprises an LCDR1 sequence comprising or consisting of a sequence of SEQ ID NO: 27, an LCDR2 sequence comprising or consisting of a sequence of SEQ ID NO: 28, and an LCDR3 sequence comprising or consisting of a sequence of SEQ ID NO: 26.

In some instances, the anti-transferrin receptor antibody comprises a VH region and a VL region, in which the VH region comprises an HCDR1 sequence comprising or consisting of a sequence of SEQ ID NO: 17, an HCDR2 sequence comprising or consisting of a sequence of SEQ ID NO: 21, and an HCDR3 sequence comprising or consisting of a sequence of SEQ ID NO: 19; and the VL region comprises an LCDR1 sequence comprising or consisting of a sequence of SEQ ID NO: 13, an LCDR2 sequence comprising or consisting of a sequence of SEQ ID NO: 23, 25, or 28, and an LCDR3 sequence comprising or consisting of a sequence of SEQ ID NO: 24 or 26, wherein X3 is selected from N or S.

In some instances, the anti-transferrin receptor antibody comprises a VH region and a VL region, in which the VH region comprises an HCDR1 sequence comprising or consisting of a sequence of SEQ ID NO: 17, an HCDR2 sequence comprising or consisting of a sequence of SEQ ID NO: 21, and an HCDR3 sequence comprising or consisting of a sequence of SEQ ID NO: 19; and the VL region comprises an LCDR1 sequence comprising or consisting of a sequence of SEQ ID NO: 22 or 27, an LCDR2 sequence comprising or consisting of a sequence of SEQ ID NO: 14, and an LCDR3 sequence comprising or consisting of a sequence of SEQ ID NO: 24 or 26, wherein X4 is selected from A or G, and X5 is selected from D or E.

In some instances, the anti-transferrin receptor antibody comprises a VH region and a VL region, in which the VH region comprises an HCDR1 sequence comprising or consisting of a sequence of SEQ ID NO: 17, an HCDR2 sequence comprising or consisting of a sequence of SEQ ID NO: 21, and an HCDR3 sequence comprising or consisting of a sequence of SEQ ID NO: 19; and the VL region comprises an LCDR1 sequence comprising or consisting of a sequence of SEQ ID NO: 22 or 27, an LCDR2 sequence comprising or consisting of a sequence of SEQ ID NO: 23, 25, or 28, and LCDR3 sequence comprising or consisting of a sequence of SEQ ID NO: 15, wherein X6 is present or absent, and if present, is F.

In some instances, the anti-transferrin receptor antibody comprises a VH region and a VL region, in which the VH region comprises an HCDR1 sequence comprising or consisting of a sequence of SEQ ID NO: 17, an HCDR2 sequence comprising or consisting of a sequence of SEQ ID NO: 21, and an HCDR3 sequence comprising or consisting of a sequence of SEQ ID NO: 19, and the VL region comprises an LCDR1 sequence comprising or consisting of a sequence of SEQ ID NO: 22, an LCDR2 sequence comprising or consisting of a sequence of SEQ ID NO: 16, and an LCDR3 sequence comprising or consisting of a sequence of SEQ ID NO: 15, wherein X5 is selected from D or E and X6 is present or absent, and if present, is F.

In some instances, the anti-transferrin receptor antibody comprises a VH region and a VL region, in which the VH region comprises an HCDR1 sequence comprising or consisting of a sequence of SEQ ID NO: 17, an HCDR2 sequence comprising or consisting of a sequence of SEQ ID NO: 21, and an HCDR3 sequence comprising or consisting of a sequence of SEQ ID NO: 19; and the VL region comprises an LCDR1 sequence comprising or consisting of a sequence of SEQ ID NO: 22, an LCDR2 sequence comprising or consisting of a sequence of SEQ ID NO: 23, and an LCDR3 sequence comprising or consisting of a sequence of SEQ ID NO: 24.

In some instances, the anti-transferrin receptor antibody comprises a VH region and a VL region, in which the VH region comprises an HCDR1 sequence comprising or consisting of a sequence of SEQ ID NO: 17, an HCDR2 sequence comprising or consisting of a sequence of SEQ ID NO: 21, and an HCDR3 sequence comprising or consisting of a sequence of SEQ ID NO: 19; and the VL region comprises an LCDR1 sequence comprising or consisting of a sequence of SEQ ID NO: 22, an LCDR2 sequence comprising or consisting of a sequence of SEQ ID NO: 25, and an LCDR3 sequence comprising or consisting of a sequence of SEQ ID NO: 26.

In some instances, the anti-transferrin receptor antibody comprises a VH region and a VL region, in which the VH region comprises an HCDR1 sequence comprising or consisting of a sequence of SEQ ID NO: 17, an HCDR2 sequence comprising or consisting of a sequence of SEQ ID NO: 21, and an HCDR3 sequence comprising or consisting of a sequence of SEQ ID NO: 19; and the VL region comprises an LCDR1 sequence comprising or consisting of a sequence of SEQ ID NO: 27, an LCDR2 sequence comprising or consisting of a sequence of SEQ ID NO: 28, and an LCDR3 sequence comprising or consisting of a sequence of SEQ ID NO: 26.

In some aspects, the anti-transferrin receptor antibody comprises a VH region and a VL region in which the sequence of the VH region comprises or consists of a sequence with about 80%, about 85%, about 90%, about 95%, about 96%, about 97%, about 98%, about 99%, or about 100% sequence identity to a sequence selected from SEQ ID NOs: 29-33 and the sequence of the VL region comprises or consisting of a sequence with about 80%, about 85%, about 90%, about 95%, about 96%, about 97%, about 98%, about 99%, or about 100% sequence identity to a sequence selected from SEQ ID NOs: 34-38. In some aspects, the anti-transferrin receptor antibody comprises a VH region and a VL region in which the sequence of the VH region comprises or consists of a sequence with at least 80%, at least 85%, at least 90%, at least 95%, at least 96%, at least 97%, at least 98%, at least 99%, or 100% sequence identity to a sequence selected from a sequence of SEQ ID NOs: 29-33 and the sequence of the VL region comprises or consists of a sequence with at least 80%, at least 85%, at least 90%, at least 95%, at least 96%, at least 97%, at least 98%, at least 99%, or 100% sequence identity to a sequence selected from a sequence of SEQ ID NOs: 34-38. In some aspects, the anti-transferrin receptor antibody comprises a VH region and a VL region in which the sequence of the VH region comprises or consists of a sequence selected from a sequence of SEQ ID NOs: 29-33 and the sequence of the VL region comprises or consists of a sequence selected from a sequence of SEQ ID NOs: 34-38.

In some aspects, the VH region comprises or consists of a sequence selected from SEQ ID NOs: 29-33 (Table 5) and the VL region comprises or consists of a sequence selected from SEQ ID NOs: 34-38 (Table 6). The underlined regions in Table 4 and Table 5 denote the respective CDR1, CDR2, or CDR3 sequence.

TABLE 5

| | | SEQ ID |
|---|---|---|
| NAME | VH SEQUENCE | NO: |
| 13E4_VH1 | QVQLVQSGAEVKKPGASVKVSCKASGYTFTNYWMHWVRQAPGQ | 29 |
| | GLEWMGEINPINGRSNYAQKFQGRVTLTVDTSISTAYMELSRLRSD | |
| | DTAVYYCARGTRAMHYWGQGTLVTVSS | |
| 13E4_VH2 | QVQLVQSGAEVKKPGASVKVSCKASGYTFTNYWMHWVRQAPGQ | 30 |
| | GLEWIGEINPINGRSNYAEKFQGRVTLTVDTSSSTAYMELSRLRSDD | |
| | TAVYYCARGTRAMHYWGQGTLVTVSS | |
| 13E4_VH3 | QVQLVQSGAEVKKPGASVKVSCKASGYTFTNYWMHWVRQAPGQ | 31 |
| | GLEWMGEINPIQGRSNYAEKFQGRVTLTVDTSSSTAYMELSSLRSE | |
| | DTATYYCARGTRAMHYWGQGTLVTVSS | |
| 13E4_VH4 | QVQLVQSGAEVKKPGASVKVSCKASGYTFTNYWMHWVRQAPGQ | 32 |
| | GLEWMGEINPINGRSNYAEKFQGRVTLTVDTSSSTAYMELSSLRSE | |
| | DTATYYCARGTRAMHYWGQGTLVTVSS | |
| 13E4_VH | QVQLQQPGAELVKPGASVKLSCKASGYTFTNYWMHWVKQRPGQG | 33 |
| | LEWIGEINPINGRSNYGERFKTKATLTVDKSSSTAYMQLSSLTSEDS | |
| | AVYYCARGTRAMHYWGQGTSVTVSS | |

TABLE 6

| | | SEQ ID |
|---|---|---|
| NAME | VL SEQUENCE | NO: |
| 13E4_VL1 | DIQMTQSPSSLSASVGDRVTITCRTSENIYNNLAWYQQKPGKSPKLL | 34 |
| | IYAATNLADGVPSRFSGSGSGTDYTLTISSLQPEDFATYYCQHFWGT | |
| | PLTFGGGTKVEIK | |
| 13E4_VL2 | DIQMTQSPSSLSASVGDRVTITCRTSENIYNNLAWYQQKPGKAPKLL | 35 |
| | IYAATNLADGVPSRFSGSGSGTDYTLTISSLQPEDFATYYCQHFWGT | |
| | PLTFGGGTKVEIK | |
| 13E4_VL3 | DIQMTQSPSSLSASVGDRVTITCRTSENIYNNLAWYQQKPGKAPKLL | 36 |
| | IYAATNLAEGVPSRFSGSGSGTDYTLTISSLQPEDFATYYCQHFWGT | |
| | PLTFGGGTKVEIK | |
| 13E4_VL4 | DIQMTQSPSSLSASVGDRVTITCRTSENIYSNLAWYQQKPGKAPKLL | 37 |
| | IYAGTNLADGVPSRFSGSGSGTDYTLTISSLQPEDFANYYCQHFWGT | |
| | PLTFGGGTKVEIK | |
| 13E4_VL | DIQMTQSPASLSVSVGETVTITCRTSENIYNNLAWYQQKQGKSPQLL | 38 |
| | VYAATNLADGVPSRFSGSGSGTQYSLKINSLQSEDFGNYYCQHFWG | |
| | TPLTFGAGTKLELK | |

In some aspects, the anti-transferrin receptor antibody comprises a VH region and a VL region as illustrated in Table 7.

TABLE 7

| | 13E4_VH1 | 13E4_VH2 | 13E4_VH3 | 13E4_VH4 |
|---|---|---|---|---|
| | (SEQ ID NO: 29) | (SEQ ID NO: 30) | (SEQ ID NO: 31) | (SEQ ID NO: 32) |
| 13E4_VL1 | SEQ ID NO: 29 + | SEQ ID NO: 30 + | SEQ ID NO: 31 + | SEQ ID NO: 32 + |
| (SEQ ID NO: 34) | SEQ ID NO: 34 | SEQ ID NO: 34 | SEQ ID NO: 34 | SEQ ID NO: 34 |
| 13E4_VL2 | SEQ ID NO: 29 + | SEQ ID NO: 30 + | SEQ ID NO: 31 + | SEQ ID NO: 32 + |
| (SEQ ID NO: 35) | SEQ ID NO: 35 | SEQ ID NO: 35 | SEQ ID NO: 35 | SEQ ID NO: 35 |
| 13E4_VL3 | SEQ ID NO: 29 + | SEQ ID NO: 30 + | SEQ ID NO: 31 + | SEQ ID NO: 32 + |
| (SEQ ID NO: 36) | SEQ ID NO: 36 | SEQ ID NO: 36 | SEQ ID NO: 36 | SEQ ID NO: 36 |
| 13E4_VL4 | SEQ ID NO: 29 + | SEQ ID NO: 30 + | SEQ ID NO: 31 + | SEQ ID NO: 32 + |
| (SEQ ID NO: 37) | SEQ ID NO: 37 | SEQ ID NO: 37 | SEQ ID NO: 37 | SEQ ID NO: 37 |

In some aspects, an anti-transferrin receptor antibody described herein comprises an IgG framework, an IgA framework, an IgE framework, or an IgM framework. In some instances, the anti-transferrin receptor antibody comprises an IgG framework (e.g., IgG1, IgG2, IgG3, or IgG4). In some cases, the anti-transferrin receptor antibody comprises an IgG1 framework. In some cases, the anti-transferrin receptor antibody comprises an IgG2 (e.g., an IgG2a or IgG2b) framework. In some cases, the anti-transferrin receptor antibody comprises an IgG2a framework. In some cases, the anti-transferrin receptor antibody comprises an IgG2b framework. In some cases, the anti-transferrin receptor antibody comprises an IgG3 framework. In some cases, the anti-transferrin receptor antibody comprises an IgG4 framework.

In some cases, an anti-transferrin receptor antibody comprises one or more mutations in a framework region, e.g., in the CH1 domain, CH2 domain, CH3 domain, hinge region, or a combination thereof. In some instances, the one or more mutations are to stabilize the antibody and/or to increase half-life. In some instances, the one or more mutations are to modulate Fc receptor interactions, to reduce or eliminate Fc effector functions such as FcyR, antibody-dependent cell-mediated cytotoxicity (ADCC), or complement-dependent cytotoxicity (CDC). In additional instances, the one or more mutations are to modulate glycosylation.

In some aspects, the one or more mutations are located in the Fc region. In some instances, the Fc region comprises a mutation at residue position L234, L235, or a combination thereof. In some instances, the mutations comprise L234 and L235. In some instances, the mutations comprise L234A and L235A. In some cases, the residue positions are in reference to IgG1.

In some instances, the Fc region comprises a mutation at residue position L234, L235, D265, N21, K46, L52, or P53, or a combination thereof. In some instances, the mutations comprise L234 and L235 in combination with a mutation at residue position K46, L52, or P53. In some cases, the Fc region comprises mutations at L234, L235, and K46. In some cases, the Fc region comprises mutations at L234, L235, and L52. In some cases, the Fc region comprises mutations at L234, L235, and P53. In some cases, the Fc region comprises mutations at D265 and N21. In some cases, the residue position is in reference to IgG1.

In some instances, the Fc region comprises L234A, L235A, D265A, N21G, K46G, L52R, or P53G, or a combination thereof. In some instances, the Fc region comprises L234A and L235A in combination with K46G, L52R, or P53G. In some cases, the Fc region comprises L234A, L235A, and K46G. In some cases, the Fc region comprises L234A, L235A, and L52R. In some cases, the Fc region comprises L234A, L235A, and P53G. In some cases, the Fc region comprises D265A and N21G. In some cases, the residue position is in reference to IgG1.

In some instances, the Fc region comprises a mutation at residue position L235, L236, D265, N21, K46, L52, or P53, or a combination of the mutations. In some instances, the Fc region comprises mutations at L235 and L236. In some instances, the Fc region comprises mutations at L235 and L236 in combination with a mutation at residue position K46, L52, or P53. In some cases, the Fc region comprises mutations at L235, L236, and K46. In some cases, the Fc region comprises mutations at L235, L236, and L52. In some cases, the Fc region comprises mutations at L235, L236, and P53. In some cases, the Fc region comprises mutations at D265 and N21. In some cases, the residue position is in reference to IgG2b.

In some aspects, the Fc region comprises L235A, L236A, D265A, N21G, K46G, L52R, or P53G, or a combination thereof. In some instances, the Fc region comprises L235A and L236A. In some instances, the Fc region comprises L235A and L236A in combination with K46G, L52R, or P53G. In some cases, the Fc region comprises L235A, L236A, and K46G. In some cases, the Fc region comprises L235A, L236A, and L52R. In some cases, the Fc region comprises L235A, L236A, and P53G. In some cases, the Fc region comprises D265A and N21G. In some cases, the residue position is in reference to IgG2b.

In some aspects, the Fc region comprises a mutation at residue position L233, L234, D264, N20, K45, L51, or P52, wherein the residues correspond to positions 233, 234, 264, 20, 45, 51, and 52 of SEQ ID NO: 39. In some instances, the Fc region comprises mutations at L233 and L234. In some instances, the Fc region comprises mutations at L233 and L234 in combination with a mutation at residue position K45, L51, or P52. In some cases, the Fc region comprises mutations at L233, L234, and K45. In some cases, the Fc region comprises mutations at L233, L234, and L51. In some cases, the Fc region comprises mutations at L233, L234, and K45. In some cases, the Fc region comprises mutations at L233, L234, and P52. In some instances, the Fc region comprises mutations at D264 and N20. In some cases, equivalent positions to residue L233, L234, D264, N20, K45, L51, or P52 in an IgG1, IgG2, IgG3, or IgG4 framework are contemplated. In some cases, mutations to a residue that corresponds to residue L233, L234, D264, N20, K45, L51, or P52 of SEQ ID NO: 39 in an IgG1, IgG2, or IgG4 framework are also contemplated.

In some aspects, the Fc region comprises L233A, L234A, D264A, N20G, K45G, L51R, or P52G, wherein the residues correspond to positions 233, 234, 264, 20, 45, 51, and 52 of SEQ ID NO: 39. In some instances, the Fc region comprises L233A and L234A. In some instances, the Fc region comprises L233A and L234A in combination with K45G, L51R, or P52G. In some cases, the Fc region comprises L233A, L234A, and K45G. In some cases, the Fc region comprises L233A, L234A, and L51R. In some cases, the Fc region comprises L233A, L234A, and K45G. In some cases, the Fc region comprises L233A, L234A, and P52G. In some instances, the Fc region comprises D264A and N20G.

In some aspects, the human IgG constant region is modified to alter antibody-dependent cellular cytotoxicity (ADCC) and/or complement-dependent cytotoxicity (CDC), e.g., with an amino acid modification described in Natsume et al., 2008 Cancer Res, 68(10): 3863-72; Idusogie et al., 2001 J Immunol, 166(4): 2571-5; Moore et al., 2010 mAbs, 2(2): 181-189; Lazar et al., 2006 PNAS, 103(11): 4005-4010, Shields et al., 2001 JBC, 276(9): 6591-6604; Stavenhagen et al., 2007 Cancer Res, 67(18): 8882-8890; Stavenhagen et al., 2008 Advan. Enzyme Regul., 48: 152-164; Alegre et al, 1992 J Immunol, 148: 3461-3468; Reviewed in Kaneko and Niwa, 2011 Biodrugs, 25(1): 1-11.

In some aspects, an anti-transferrin receptor antibody described herein is a full-length antibody, comprising a heavy chain (HC) and a light chain (LC). In some cases, the heavy chain (HC) comprises a sequence selected from Table 8. In some cases, the light chain (LC) comprises a sequence selected from Table 9. The underlined region denotes the respective CDRs.

TABLE 8

| | | SEQ ID |
|---|---|---|
| NAME | HC SEQUENCE | NO: |
| 13E4_VH1 | QVQLVQSGAEVKKPGASVKVSCKASGYTFTNYWMHWVRQAPG | 39 |
| | QGLEWMGEINPINGRSNYAQKFQGRVTLTVDTSISTAYMELSRL | |
| | RSDDTAVYYCARGTRAMHYWGQGTLVTVSSASTKGPSVFPLAP | |
| | SSKSTSGGTAALGCLVKDYFPEPVTVSWNSGALTSGVHTFPAVL | |
| | QSSGLYSLSSVVTVPSSSLGTQTYICNVNHKPSNTKVDKRVEPKS | |
| | CDKTHTCPPCPAPELLGGPSVFLFPPKPKDTLMISRTPEVTCVVV | |
| | DVSHEDPEVKFNWYVDGVEVHNAKTKPREEQYNSTYRVVSVLT | |
| | VLHQDWLNGKEYKCKVSNKALPAPIEKTISKAKGQPREPQVYTL | |
| | PPSREEMTKNQVSLTCLVKGFYPSDIAVEWESNGQPENNYKTTP | |
| | PVLDSDGSFFLYSKLTVDKSRWQQGNVFSCSVMHEALHNHYTQ | |
| | KSLSLSPG | |
| 13E4_VH1_a | QVQLVQSGAEVKKPGASVKVSCKASGYTFTNYWMHWVRQAPG | 40 |
| | QGLEWMGEINPINGRSNYAQKFQGRVTLTVDTSISTAYMELSRL | |
| | RSDDTAVYYCARGTRAMHYWGQGTLVTVSSASTKGPSVFPLAP | |
| | SSKSTSGGTAALGCLVKDYFPEPVTVSWNSGALTSGVHTFPAVL | |
| | QSSGLYSLSSVVTVPSSSLGTQTYICNVNHKPSNTKVDKRVEPKS | |
| | CDKTHTCPPCPAPEAAGGPSVFLFPPKPKDTLMISRTPEVTCVVV | |
| | DVSHEDPEVKFNWYVDGVEVHNAKTKPREEQYNSTYRVVSVLT | |
| | VLHQDWLNGKEYKCKVSNKALPAPIEKTISKAKGQPREPQVYTL | |
| | PPSREEMTKNQVSLTCLVKGFYPSDIAVEWESNGQPENNYKTTP | |
| | PVLDSDGSFFLYSKLTVDKSRWQQGNVFSCSVMHEALHNHYTQ | |
| | KSLSLSPG | |
| 13E4_VH1_b | QVQLVQSGAEVKKPGASVKVSCKASGYTFTNYWMHWVRQAPG | 41 |
| | QGLEWMGEINPINGRSNYAQKFQGRVTLTVDTSISTAYMELSRL | |
| | RSDDTAVYYCARGTRAMHYWGQGTLVTVSSASTKGPSVFPLAP | |
| | SSKSTSGGTAALGCLVKDYFPEPVTVSWNSGALTSGVHTFPAVL | |
| | QSSGLYSLSSVVTVPSSSLGTQTYICNVNHKPSNTKVDKRVEPKS | |
| | CDKTHTCPPCPAPEAAGGPSVFLFPPKPKDTLMISRTPEVTCVVV | |
| | DVSHEDPEVKFNWYVDGVEVHNAKTKPREEQYNSTYRVVSVLT | |
| | VLHQDWLNGKEYKCGVSNKALPAPIEKTISKAKGQPREPQVYTL | |
| | PPSREEMTKNQVSLTCLVKGFYPSDIAVEWESNGQPENNYKTTP | |
| | PVLDSDGSFFLYSKLTVDKSRWQQGNVFSCSVMHEALHNHYTQ | |
| | KSLSLSPG | |
| 13E4_VH1_c | QVQLVQSGAEVKKPGASVKVSCKASGYTFTNYWMHWVRQAPG | 42 |
| | QGLEWMGEINPINGRSNYAQKFQGRVTLTVDTSISTAYMELSRL | |
| | RSDDTAVYYCARGTRAMHYWGQGTLVTVSSASTKGPSVFPLAP | |
| | SSKSTSGGTAALGCLVKDYFPEPVTVSWNSGALTSGVHTFPAVL | |
| | QSSGLYSLSSVVTVPSSSLGTQTYICNVNHKPSNTKVDKRVEPKS | |
| | CDKTHTCPPCPAPEAAGGPSVFLFPPKPKDTLMISRTPEVTCVVV | |
| | DVSHEDPEVKFNWYVDGVEVHNAKTKPREEQYNSTYRVVSVLT | |
| | VLHQDWLNGKEYKCKVSNKARPAPIEKTISKAKGQPREPQVYTL | |
| | PPSREEMTKNQVSLTCLVKGFYPSDIAVEWESNGQPENNYKTTP | |
| | PVLDSDGSFFLYSKLTVDKSRWQQGNVFSCSVMHEALHNHYTQ | |
| | KSLSLSPG | |
| 13E4_VH1 d | QVQLVQSGAEVKKPGASVKVSCKASGYTFTNYWMHWVRQAPG | 43 |
| | QGLEWMGEINPINGRSNYAQKFQGRVTLTVDTSISTAYMELSRL | |
| | RSDDTAVYYCARGTRAMHYWGQGTLVTVSSASTKGPSVFPLAP | |
| | SSKSTSGGTAALGCLVKDYFPEPVTVSWNSGALTSGVHTFPAVL | |
| | QSSGLYSLSSVVTVPSSSLGTQTYICNVNHKPSNTKVDKRVEPKS | |
| | CDKTHTCPPCPAPEAAGGPSVFLFPPKPKDTLMISRTPEVTCVVV | |
| | DVSHEDPEVKFNWYVDGVEVHNAKTKPREEQYNSTYRVVSVLT | |
| | VLHQDWLNGKEYKCKVSNKALGAPIEKTISKAKGQPREPQVYT | |
| | LPPSREEMTKNQVSLTCLVKGFYPSDIAVEWESNGQPENNYKTT | |
| | PPVLDSDGSFFLYSKLTVDKSRWQQGNVFSCSVMHEALHNHYT | |
| | QKSLSLSPG | |
| 13E4_VH1_e | QVQLVQSGAEVKKPGASVKVSCKASGYTFTNYWMHWVRQAPG | 44 |
| | QGLEWMGEINPINGRSNYAQKFQGRVTLTVDTSISTAYMELSRL | |
| | RSDDTAVYYCARGTRAMHYWGQGTLVTVSSASTKGPSVFPLAP | |
| | SSKSTSGGTAALGCLVKDYFPEPVTVSWNSGALTSGVHTFPAVL | |
| | QSSGLYSLSSVVTVPSSSLGTQTYICNVNHKPSNTKVDKRVEPKS | |
| | CDKTHTCPPCPAPELLGGPSVFLFPPKPKDTLMISRTPEVTCVVV | |
| | AVSHEDPEVKFNWYVDGVEVHNAKTKPREEQYGSTYRVVSVLT | |
| | VLHQDWLNGKEYKCKVSNKALPAPIEKTISKAKGQPREPQVYTL | |
| | PPSREEMTKNQVSLTCLVKGFYPSDIAVEWESNGQPENNYKTTP | |
| | PVLDSDGSFFLYSKLTVDKSRWQQGNVFSCSVMHEALHNHYTQ | |
| | KSLSLSPG | |
| 13E4_VH2 | QVQLVQSGAEVKKPGASVKVSCKASGYTFTNYWMHWVRQAPG | 45 |
| | QGLEWIGEINPINGRSNYAEKFQGRVTLTVDTSSSTAYMELSRLR | |
| | SDDTAVYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPELLGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCKVSNKALPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH2 a | QVQLVQSGAEVKKPGASVKVSCKASGYTFTNYWMHWVRQAPG | 46 |
| | QGLEWIGEINPINGRSNYAEKFQGRVTLTVDTSSSTAYMELSRLR | |
| | SDDTAVYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPEAAGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCKVSNKALPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH2_b | QVQLVQSGAEVKKPGASVKVSCKASGYTFTNYWMHWVRQAPG | 47 |
| | QGLEWIGEINPINGRSNYAEKFQGRVTLTVDTSSSTAYMELSRLR | |
| | SDDTAVYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPEAAGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCGVSNKALPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH2_c | QVQLVQSGAEVKKPGASVKVSCKASGYTFTNYWMHWVRQAPG | 48 |
| | QGLEWIGEINPINGRSNYAEKFQGRVTLTVDTSSSTAYMELSRLR | |
| | SDDTAVYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPEAAGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCKVSNKARPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH2 d | QVQLVQSGAEVKKPGASVKVSCKASGYTFTNYWMHWVRQAPG | 49 |
| | QGLEWIGEINPINGRSNYAEKFQGRVTLTVDTSSSTAYMELSRLR | |
| | SDDTAVYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPEAAGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCKVSNKALGAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH2 e | QVQLVQSGAEVKKPGASVKVSCKASGYTFTNYWMHWVRQAPG | 50 |
| | QGLEWIGEINPINGRSNYAEKFQGRVTLTVDTSSSTAYMELSRLR | |
| | SDDTAVYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPELLGGPSVFLFPPKPKDTLMISRTPEVTCVVVA | |
| | VSHEDPEVKFNWYVDGVEVHNAKTKPREEQYGSTYRVVSVLTV | |
| | LHQDWLNGKEYKCKVSNKALPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH3 | QVQLVQSGAEVKKPGASVKVSCKASGYTFTNYWMHWVRQAPG | 51 |
| | QGLEWMGEINPIQGRSNYAEKFQGRVTLTVDTSSSTAYMELSSL | |
| | RSEDTATYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPELLGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCKVSNKALPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH3_a | QVQLVQSGAEVKKPGASVKVSCKASGYTFTNYWMHWVRQAPG | 52 |
| | QGLEWMGEINPIQGRSNYAEKFQGRVTLTVDTSSSTAYMELSSL | |
| | RSEDTATYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPEAAGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCKVSNKALPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH3_b | QVQLVQSGAEVKKPGASVKVSCKASGYTFTNYWMHWVRQAPG | 53 |
| | QGLEWMGEINPIQGRSNYAEKFQGRVTLTVDTSSSTAYMELSSL | |
| | RSEDTATYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPEAAGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCGVSNKALPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH3_c | QVQLVQSGAEVKKPGASVKVSCKASGYTFTNYWMHWVRQAPG | 54 |
| | QGLEWMGEINPIQGRSNYAEKFQGRVTLTVDTSSSTAYMELSSL | |
| | RSEDTATYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPEAAGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCKVSNKARPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH3_d | QVQLVQSGAEVKKPGASVKVSCKASGYTFTNYWMHWVRQAPG | 55 |
| | QGLEWMGEINPIQGRSNYAEKFQGRVTLTVDTSSSTAYMELSSL | |
| | RSEDTATYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPEAAGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCKVSNKALGAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH3_e | QVQLVQSGAEVKKPGASVKVSCKASGYTFTNYWMHWVRQAPG | 56 |
| | QGLEWMGEINPIQGRSNYAEKFQGRVTLTVDTSSSTAYMELSSL | |
| | RSEDTATYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPELLGGPSVFLFPPKPKDTLMISRTPEVTCVVVA | |
| | VSHEDPEVKFNWYVDGVEVHNAKTKPREEQYGSTYRVVSVLTV | |
| | LHQDWLNGKEYKCKVSNKALPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH4 | QVQLVQSGAEVKKPGASVKVSCKASGYTFTNYWMHWVRQAPG | 57 |
| | QGLEWMGEINPINGRSNYAEKFQGRVTLTVDTSSSTAYMELSSL | |
| | RSEDTATYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPELLGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCKVSNKALPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH4 a | QVQLVQSGAEVKKPGASVKVSCKASGYTFTNYWMHWVRQAPG | 58 |
| | QGLEWMGEINPINGRSNYAEKFQGRVTLTVDTSSSTAYMELSSL | |
| | RSEDTATYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPEAAGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCKVSNKALPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH4 b | QVQLVQSGAEVKKPGASVKVSCKASGYTFTNYWMHWVRQAPG | 59 |
| | QGLEWMGEINPINGRSNYAEKFQGRVTLTVDTSSSTAYMELSSL | |
| | RSEDTATYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPEAAGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCGVSNKALPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH4_c | QVQLVQSGAEVKKPGASVKVSCKASGYTFTNYWMHWVRQAPG | 60 |
| | QGLEWMGEINPINGRSNYAEKFQGRVTLTVDTSSSTAYMELSSL | |
| | RSEDTATYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPEAAGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCKVSNKARPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH4_d | QVQLVQSGAEVKKPGASVKVSCKASGYTFTNYWMHWVRQAPG | 61 |
| | QGLEWMGEINPINGRSNYAEKFQGRVTLTVDTSSSTAYMELSSL | |
| | RSEDTATYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPEAAGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCKVSNKALGAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |
| 13E4_VH4_e | QVQLVQSGAEVKKPGASVKVSCKASGYTFTNYWMHWVRQAPG | 62 |
| | QGLEWMGEINPINGRSNYAEKFQGRVTLTVDTSSSTAYMELSSL | |
| | RSEDTATYYCARGTRAMHYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKRVEPKSC | |
| | DKTHTCPPCPAPELLGGPSVFLFPPKPKDTLMISRTPEVTCVVVA | |
| | VSHEDPEVKFNWYVDGVEVHNAKTKPREEQYGSTYRVVSVLTV | |
| | LHQDWLNGKEYKCKVSNKALPAPIEKTISKAKGQPREPQVYTLP | |
| | PSREEMTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPG | |

TABLE 9

| | | SEQ ID |
|---|---|---|
| NAME | LC SEQUENCE | NO: |
| 13E4_VL1 | DIQMTQSPSSLSASVGDRVTITCRTSENIYNNLAWYQQKPGKSPK | 63 |
| | LLIYAATNLADGVPSRFSGSGSGTDYTLTISSLQPEDFATYYCQHF | |
| | WGTPLTFGGGTKVEIKRTVAAPSVFIFPPSDEQLKSGTASVVCLL | |
| | NNFYPREAKVQWKVDNALQSGNSQESVTEQDSKDSTYSLSSTLT | |
| | LSKADYEKHKVYACEVTHQGLSSPVTKSFNRGEC | |
| 13E4_VL2 | DIQMTQSPSSLSASVGDRVTITCRTSENIYNNLAWYQQKPGKAPK | 64 |
| | LLIYAATNLADGVPSRFSGSGSGTDYTLTISSLQPEDFATYYCQHF | |
| | WGTPLTFGGGTKVEIKRTVAAPSVFIFPPSDEQLKSGTASVVCLL | |
| | NNFYPREAKVQWKVDNALQSGNSQESVTEQDSKDSTYSLSSTLT | |
| | LSKADYEKHKVYACEVTHQGLSSPVTKSFNRGEC | |
| 13E4_VL3 | DIQMTQSPSSLSASVGDRVTITCRTSENIYNNLAWYQQKPGKAPK | 65 |
| | LLIYAATNLAEGVPSRFSGSGSGTDYTLTISSLQPEDFATYYCQHF | |
| | WGTPLTFGGGTKVEIKRTVAAPSVFIFPPSDEQLKSGTASVVCLL | |
| | NNFYPREAKVQWKVDNALQSGNSQESVTEQDSKDSTYSLSSTLT | |
| | LSKADYEKHKVYACEVTHQGLSSPVTKSFNRGEC | |
| 13E4_VL4 | DIQMTQSPSSLSASVGDRVTITCRTSENIYSNLAWYQQKPGKAPK | 66 |
| | LLIYAGTNLADGVPSRFSGSGSGTDYTLTISSLQPEDFANYYCQH | |
| | FWGTPLTFGGGTKVEIKRTVAAPSVFIFPPSDEQLKSGTASVVCL | |
| | LNNFYPREAKVQWKVDNALQSGNSQESVTEQDSKDSTYSLSSTL | |
| | TLSKADYEKHKVYACEVTHQGLSSPVTKSFNRGEC | |

In some aspects, an anti-transferrin receptor antibody described herein has an improved serum half-life compared to a reference anti-transferrin receptor antibody. In some instances, the improved serum half-life is at least 30 minutes, 1 hour, 1.5 hours, 2 hours, 3 hours, 4 hours, 5 hours, 6 hours, 7 hours, 8 hours, 9 hours, 10 hours, 12 hours, 18 hours, 24 hours, 2 days, 3 days, 4 days, 5 days, 6 days, 7 days, 14 days, 30 days, or longer than the reference anti-transferrin receptor antibody.

In some aspects, an antibody or antigen binding fragment thereof is further modified using conventional techniques known in the art, for example, by using amino acid deletion, insertion, substitution, addition, and/or by recombination and/or any other modification (e.g., posttranslational and chemical modifications, such as glycosylation and phosphorylation) known in the art either alone or in combination. In some instances, the modification further comprises a modification for modulating interaction with Fc receptors. In some instances, the one or more modifications include those described in, for example, International Publication No. WO97/34631, which discloses amino acid residues involved in the interaction between the Fc domain and the FcRn receptor. Methods for introducing such modifications in the nucleic acid sequence underlying the amino acid sequence of an antibody or its binding fragment is well known to the person skilled in the art.

In some instances, an antibody or antigen binding fragment thereof further encompasses its derivatives and includes polypeptide sequences containing at least one CDR.

In some instances, the term “single-chain” as used herein means that the first and second domains of a bi-specific single chain construct are covalently linked, preferably in the form of a co-linear amino acid sequence encodable by a single nucleic acid molecule.

In some instances, a bispecific single chain antibody construct relates to a construct comprising two antibody derived binding domains. In such embodiments, the bi-specific single chain antibody construct is tandem to a bi-scFv or diabody. In some instances, a scFv contains a VH and VL domain connected by a linker peptide. In some instances, linkers are of a length and sequence sufficient to ensure that each of the first and second domains can, independently from one another, retain their differential binding specificities.

In some aspects, binding to or interacting with as used herein defines a binding/interaction of at least two antigen-interaction-sites with each other. In some instances, antigen-interaction-site defines a motif of a polypeptide that shows the capacity of specific interaction with a specific antigen or a specific group of antigens. In some cases, the binding/interaction is also understood to define a specific recognition. In such cases, specific recognition refers to whether the antibody or its binding fragment is capable of specifically interacting with and/or binding to at least two amino acids of each of a target molecule. For example, specific recognition relates to the specificity of the antibody molecule, or to its ability to discriminate between the specific regions of a target molecule. In additional instances, the specific interaction of the antigen-interaction-site with its specific antigen results in an initiation of a signal, e.g. due to the induction of a change of the conformation of the antigen, an oligomerization of the antigen, etc. In further aspects, the binding is exemplified by the specificity of a “key-lock-principle”. Thus in some instances, specific motifs in the amino acid sequence of the antigen-interaction-site and the antigen bind to each other as a result of their primary, secondary or tertiary structure as well as the result of secondary modifications of said structure. In such cases, the specific interaction of the antigen-interaction-site with its specific antigen results as well as in a simple binding of the site to the antigen.

In some instances, specific interaction further refers to a reduced cross-reactivity of the antibody or its binding fragment or a reduced off-target effect. For example, the antibody or antigen binding fragment thereof that binds to the polypeptide/protein of interest but do not or do not essentially bind to any of the other polypeptides are considered as specific for the polypeptide/protein of interest. Examples for the specific interaction of an antigen-interaction-site with a specific antigen comprise the specificity of a ligand for its receptor, for example, the interaction of an antigenic determinant (epitope) with the antigenic binding site of an antibody.

### Production of Antibodies or Antigen Binding Fragments Thereof

In some aspects, polypeptides described herein (e.g., antibodies and antigen binding fragments) are produced using any method known in the art to be useful for the synthesis of polypeptides (e.g., antibodies), in particular, by chemical synthesis or by recombinant expression, and are preferably produced by recombinant expression techniques.

In some instances, an antibody or antigen binding fragment thereof is expressed recombinantly, and the nucleic acid encoding the antibody or antigen binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or antigen binding fragment thereof is optionally generated by immunizing an animal, such as a rabbit, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some aspects, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region, e.g., humanized antibodies.

In some aspects, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some aspects, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific aspects, the expression of the antibody is regulated by a constitutive, an inducible or a tissue-specific promoter.

In some aspects, a variety of host-expression vector systems is utilized to express an antibody or antigen binding fragment thereof described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

For long-term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk−, hgprt− or aprt− cells, respectively. Also, antimetabolite resistance are used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some instances, the expression levels of an antibody are increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification or analysis of an antibody or antibody conjugates is used, for example, by chromatography (e.g., ion exchange, affinity, particularly by affinity for the specific antigen after Protein A, and sizing column chromatography), centrifugation, differential solubility, or by any other standard technique for the purification of proteins. Exemplary chromatography methods included, but are not limited to, strong anion exchange chromatography, hydrophobic interaction chromatography, size exclusion chromatography, and fast protein liquid chromatography.

### Binding Moiety-Oligonucleotide Conjugate/Antibody-PMO Conjugate

In some instances, the oligonucleotide conjugate described herein comprises an antibody or antigen binding fragment thereof conjugated to an oligonucleotide molecule. In some instances, the oligonucleotide molecule can be ASO or PMO. In some instances, the one or more oligonucleotide molecule is PMO. In some instances, The antibody can be an anti-transferrin receptor (anti-CD71) antibody or antigen binding fragment thereof.

In some aspects, the antibody or antigen binding fragment thereof is conjugated to an oligonucleotide molecule non-specifically. In some instances, the antibody or antigen binding fragment thereof is conjugated to an oligonucleotide molecule via a lysine residue. In some instances, the antibody or antigen binding fragment thereof is conjugated to an oligonucleotide molecule via a cysteine residue. In some instances, the antibody or antigen binding fragment thereof is conjugated to an oligonucleotide molecule via a lysine residue or a cysteine residue, in a non-site specific manner. In some instances, the antibody or antigen binding fragment thereof is conjugated to an oligonucleotide molecule via a lysine residue (e.g., lysine residue present in the antibody in a non-site specific manner. In some cases, the antibody or antigen binding fragment thereof is conjugated to an oligonucleotide molecule via a cysteine residue (e.g., cysteine residue present in the antibody in a non-site specific manner.

In some aspects, the antibody is conjugated to an oligonucleotide molecule in a site-specific manner. In some instances, the antibody is conjugated to an oligonucleotide molecule through a lysine residue, a cysteine residue, at the 5′-terminus, at the 3′-terminus, an unnatural amino acid, or an enzyme-modified or enzyme-catalyzed residue, via a site-specific manner. In some instances, the antibody is conjugated to an oligonucleotide molecule through a lysine residue (e.g., lysine residue present in the antibody via a site-specific manner). In some instances, the antibody is conjugated to an oligonucleotide molecule through a cysteine residue (e.g., cysteine residue present in the antibody via a site-specific manner). In some instances, the antibody is conjugated to an oligonucleotide molecule at the 5′-terminus via a site-specific manner. In some instances, the antibody is conjugated to a polynucleic acid molecule at the 3′-terminus via a site-specific manner. In some instances, the antibody is conjugated to an oligonucleotide molecule through an unnatural amino acid via a site-specific manner. In some instances, the antibody is conjugated to an oligonucleotide molecule through an enzyme-modified or enzyme-catalyzed residue via a site-specific manner. In some instances, the antibody is conjugated to a polynucleic acid molecule via a linker or one or more linkers.

In some aspects, the antibody or antigen binding fragment thereof is conjugated to any of the oligonucleotide (e.g., PMO) molecules disclosed herein in a site-specific manner. In some instances, the antibody or antigen binding fragment thereof is conjugated to any of the oligonucleotide (e.g., PMO) molecules disclosed herein through a lysine residue, a cysteine residue, at the 5′-terminus, at the 3′-terminus, an unnatural amino acid, or an enzyme-modified or enzyme-catalyzed residue, via a site-specific manner. In some instances, the antibody or antigen binding fragment thereof is conjugated to any of the oligonucleotide (e.g., PMO) molecules disclosed herein through a lysine residue via a site-specific manner. In some instances, the antibody or antigen binding fragment thereof is conjugated to any of the oligonucleotide (e.g., PMO) molecules disclosed herein through a cysteine residue via a site-specific manner. In some instances, the antibody or antigen binding fragment thereof is conjugated to any of the oligonucleotide (e.g., PMO) molecules disclosed herein at the 5′-terminus via a site-specific manner. In some instances, the antibody or antigen binding fragment thereof is conjugated to any of the oligonucleotide (e.g., PMO) molecules disclosed herein at the 3′-terminus via a site-specific manner. In some instances, the antibody or antigen binding fragment thereof is conjugated to any of the oligonucleotide (e.g., PMO) molecules disclosed herein through an unnatural amino acid via a site-specific manner. In some instances, the antibody or antigen binding fragment thereof is conjugated to any of the oligonucleotide (e.g., PMO) molecules disclosed herein through an enzyme-modified or enzyme-catalyzed residue via a site-specific manner.

In some aspects, one or more oligonucleotides are conjugated to an antibody. The one or more oligonucleotides can be ASOs or PMOs. In some instances, the one or more oligonucleotides are PMOs. The antibody can be an anti-transferrin receptor (anti-CD71) antibody or antigen binding fragment thereof. In some cases, the one or more oligonucleotides are the same. In other cases, the one or more oligonucleotides are different.

In some aspects, one or more oligonucleotide (e.g., PMO) molecule is conjugated to any of the antibodies or antigen binding fragments thereof disclosed herein. In some instances, about 1, 2, 3, 4, 5, 6, 7, 8, 9, 10, 11, 12, 13, 14, 15, 16, or more oligonucleotide (e.g., PMO) molecules are conjugated to an antibody or antigen binding fragment thereof. In some instances, about 1 oligonucleotide (e.g., PMO) molecule is conjugated to one antibody or antigen binding fragment thereof. In some instances, about 2 oligonucleotide (e.g., PMO) molecules are conjugated to one antibody or antigen binding fragment thereof. In some instances, about 3 oligonucleotide (e.g., PMO) molecules are conjugated to one antibody or antigen binding fragment thereof. In some instances, about 4 oligonucleotide (e.g., PMO) molecules are conjugated to one. In some instances, about 5 oligonucleotide (e.g., PMO) molecules are conjugated to one antibody or antigen binding fragment thereof. In some instances, about 6 oligonucleotide (e.g., PMO) molecules are conjugated to one antibody or antigen binding fragment thereof. In some instances, about 7 oligonucleotide (e.g., PMO) molecules are conjugated to one antibody or antigen binding fragment thereof. In some instances, about 8 oligonucleotide (e.g., PMO) molecules are conjugated to one antibody or antigen binding fragment thereof.

In some aspects, at least 1, 2, 3, 4, 5, 6, 7, 8, 9, 10, 11, 12, 13, 14, 15, 16, 17, 18, 19, or 20 oligonucleotide (e.g., PMO) molecules are conjugated to an antibody or antigen binding fragment thereof. In some instances, at least 1 oligonucleotide (e.g., PMO) molecule is conjugated to one antibody or antigen binding fragment thereof. In some instances, at least 2 oligonucleotide (e.g., PMO) molecules are conjugated to one antibody or antigen binding fragment thereof. In some instances, at least 3 oligonucleotide (e.g., PMO) molecules are conjugated to one antibody or antigen binding fragment thereof. In some instances, at least 4 oligonucleotide (e.g., PMO) molecules are conjugated to one. In some instances, at least 5 oligonucleotide (e.g., PMO) molecules are conjugated to one antibody or antigen binding fragment thereof. In some instances, at least 6 oligonucleotide (e.g., PMO) molecules are conjugated to one antibody or antigen binding fragment thereof. In some instances, at least 7 oligonucleotide (e.g., PMO) molecules are conjugated to one antibody or antigen binding fragment thereof. In some instances, at least 8 oligonucleotide (e.g., PMO) molecules are conjugated to one antibody or antigen binding fragment thereof.

In some instances, from about 1 to about 16 oligonucleotide (e.g., PMO) molecules are conjugated to an antibody or antigen binding fragment thereof. In some instances, from about 2 to about 15 oligonucleotide (e.g., PMO) molecules are conjugated to an antibody or antigen binding fragment thereof. In some instances, from about 3 to about 14 oligonucleotide (e.g., PMO) molecules are conjugated to an antibody or antigen binding fragment thereof. In some instances, from about 4 to about 13 oligonucleotide (e.g., PMO) molecules are conjugated to an antibody or antigen binding fragment thereof. In some instances, from about 5 to about 12 oligonucleotide (e.g., PMO) molecules are conjugated to an antibody or antigen binding fragment thereof. In some instances, from about 6 to about 11 oligonucleotide (e.g., PMO) molecules are conjugated to an antibody or antigen binding fragment thereof. In some instances, from about 7 to about 10 oligonucleotide (e.g., PMO) molecules are conjugated to an antibody or antigen binding fragment thereof. In some instances, from about 8 to about 9 oligonucleotide (e.g., PMO) molecules are conjugated to an antibody or antigen binding fragment thereof.

In some aspects, an average of one or more oligonucleotide (e.g., PMO) molecule is conjugated to an antibody or antigen binding fragment thereof. In some instances, an average of about 1, 2, 3, 4, 5, 6, 7, 8, 9, 10, 11, 12, 13, 14, 15, 16, or more oligonucleotide (e.g., PMO) molecules are conjugated to an antibody or antigen binding fragment thereof. In some instances, an average of about 1 oligonucleotide (e.g., PMO) molecule is conjugated to one antibody or antigen binding fragment thereof. In some instances, an average of about 2 oligonucleotide (e.g., PMO) molecules are conjugated to one antibody or antigen binding fragment thereof. In some instances, an average of about 3 oligonucleotide (e.g., PMO) molecules are conjugated to one antibody or antigen binding fragment thereof. In some instances, an average of about 4 oligonucleotide (e.g., PMO) molecules are conjugated to one. In some instances, an average of about 5 oligonucleotide (e.g., PMO) molecules are conjugated to one antibody or antigen binding fragment thereof. In some instances, an average of about 6 oligonucleotide (e.g., PMO) molecules are conjugated to one antibody or antigen binding fragment thereof. In some instances, an average of about 7 oligonucleotide (e.g., PMO) molecules are conjugated to one antibody or antigen binding fragment thereof. In some instances, an average of about 8 oligonucleotide (e.g., PMO) molecules are conjugated to one antibody or antigen binding fragment thereof.

In some instances, an average of at least 1, 2, 3, 4, 5, 6, 7, 8, 9, 10, 11, 12, 13, 14, 15, 16, or 17 oligonucleotide (e.g., PMO) molecules are conjugated to an antibody or antigen binding fragment thereof. In some instances, an average of at least 1 oligonucleotide (e.g., PMO) molecule is conjugated to one antibody or antigen binding fragment thereof. In some instances, an average of at least 2 oligonucleotide (e.g., PMO) molecules are conjugated to one antibody or antigen binding fragment thereof. In some instances, an average of at least 3 oligonucleotide (e.g., PMO) molecules are conjugated to one antibody or antigen binding fragment thereof. In some instances, an average of at least 4 oligonucleotide (e.g., PMO) molecules are conjugated to one antibody or antigen binding fragment thereof. In some instances, an average of at least 5 oligonucleotide (e.g., PMO) molecules are conjugated to one antibody or antigen binding fragment thereof. In some instances, an average of at least 6 oligonucleotide (e.g., PMO) molecules are conjugated to one antibody or antigen binding fragment thereof. In some instances, an average of at least 7 oligonucleotide (e.g., PMO) molecules are conjugated to one antibody or antigen binding fragment thereof. In some instances, an average of at least 8 oligonucleotide (e.g., PMO) molecules are conjugated to one antibody or antigen binding fragment thereof.

In some aspects, the number of oligonucleotide molecule conjugated to an antibody forms a ratio. In some instances, the ratio is referred to as a DAR (drug-to-antibody) ratio, in which the drug as referred to herein is the oligonucleotide molecule. In some instances, the DAR ratio of the oligonucleotide molecule to antibody is about 1, 2, 3, 4, 5, 6, 7, 8, 9, 10, 11, 12, 13, 14, 15, 16, or greater. In some instances, the DAR ratio of the oligonucleotide molecule to antibody is approximately 1, 2, 3, 4, 5, 6, 7, 8, 9, 10, 11, 12, 13, 14, 15, 16, or greater. In some instances, the DAR ratio includes whole number as well as fractions or decimal of a DAR ratio. For instance, the fractions or decimal of a DAR ratio includes X.1, X.2, X.3, X.4, X.5, X.6, X.7, X.8, X.9 (e.g., 2.1, 2.2, 2.3, 2.4, 2.5, 2.6, 2.7, 2.8, 2.9, etc.). In some instances, the DAR ratio of the oligonucleotide molecule to antibody is about 1 or greater. In some instances, the DAR ratio of the oligonucleotide molecule to antibody is about 2 or greater. In some instances, the DAR ratio of the oligonucleotide molecule to antibody is about 3 or greater. In some instances, the DAR ratio of the oligonucleotide molecule to antibody is about 4 or greater. In some instances, the DAR ratio of the oligonucleotide molecule to antibody is about 5 or greater. In some instances, the DAR ratio of the oligonucleotide molecule to antibody is about 6 or greater. In some instances, the DAR ratio of the oligonucleotide molecule to antibody is about 7 or greater. In some instances, the DAR ratio of the oligonucleotide molecule to antibody is about 8 or greater. In some instances, the DAR ratio of the oligonucleotide molecule to antibody is about 9 or greater. In some instances, the DAR ratio of the oligonucleotide molecule to antibody is about 10 or greater. In some instances, the DAR ratio of the oligonucleotide molecule to antibody is about 11 or greater. In some instances, the DAR ratio of the oligonucleotide molecule to antibody is about 12 or greater.

In some instances, the DAR ratio of the oligonucleotide molecule to antibody is about 1, 2, 3, 4, 5, 6, 7, 8, 9, 10, 11, 12, 13, 14, 15, or 16. In some instances, the DAR ratio of the oligonucleotide molecule to antibody is about 1. In some instances, the DAR ratio of the oligonucleotide molecule to antibody is about 2. In some instances, the DAR ratio of the oligonucleotide molecule to antibody is about 3. In some instances, the DAR ratio of the oligonucleotide molecule to antibody is about 4. In some instances, the DAR ratio of the oligonucleotide molecule to antibody is about 5. In some instances, the DAR ratio of the oligonucleotide molecule to antibody is about 6. In some instances, the DAR ratio of the oligonucleotide molecule to antibody is about 7. In some instances, the DAR ratio of the oligonucleotide molecule to antibody is about 8. In some instances, the DAR ratio of the oligonucleotide molecule to antibody is about 9. In some instances, the DAR ratio of the oligonucleotide molecule to antibody is about 10. In some instances, the DAR ratio of the oligonucleotide molecule to antibody is about 11. In some instances, the DAR ratio of the oligonucleotide molecule to antibody is about 12. In some instances, the DAR ratio of the oligonucleotide molecule to antibody is about 13. In some instances, the DAR ratio of the oligonucleotide molecule to antibody is about 14. In some instances, the DAR ratio of the oligonucleotide molecule to antibody is about 15. In some instances, the DAR ratio of the oligonucleotide molecule to antibody is about 16.

In some instances, the DAR ratio of the oligonucleotide molecule to antibody is 1, 2, 3, 4, 5, 6, 7, 8, 9, 10, 11, 12, 13, 14, 15, or 16. In some instances, the DAR ratio of the oligonucleotide molecule to antibody is 1. In some instances, the DAR ratio of the oligonucleotide molecule to antibody is 2. In some instances, the DAR ratio of the oligonucleotide molecule to antibody is 4. In some instances, the DAR ratio of the oligonucleotide molecule to antibody is 6. In some instances, the DAR ratio of the oligonucleotide molecule to antibody is 8. In some instances, the DAR ratio of the oligonucleotide molecule to antibody is 12. In some instances, the DAR ratio of the oligonucleotide molecule to antibody is 16.

In some aspects, a composition comprises a plurality of binding moiety (e.g., antibody)-oligonucleotide conjugates. In some instances, the number of oligonucleotide molecule conjugated to an antibody forms a ratio. In some instances, the ratio is referred to as a DAR (drug-to-antibody) ratio, in which the drug as referred to herein is the oligonucleotide molecule. In some instances, the plurality of binding moiety (e.g., antibody)-oligonucleotide conjugates in the composition has the same DAR ratio. In some instances, the plurality of binding moiety (e.g., antibody)-oligonucleotide conjugates in the composition has different DAR ratios. In some instances, at least two of the binding moiety (e.g., antibody)-oligonucleotide conjugates in the composition have different DAR ratios to each other. In some instances, the DAR ratio is an average DAR (drug-to-antibody) ratio, which is an average number of the DAR ratios of the plurality of binding moiety (e.g., antibody)-oligonucleotide conjugates in the composition. In some instances, the average DAR ratio of the oligonucleotide molecule to antibody is about 1, 2, 3, 4, 5, 6, 7, 8, 9, 10, 11, 12, 13, 14, 15, 16, or greater. In some instances, the average DAR ratio of the oligonucleotide molecule to antibody is approximately 1, 2, 3, 4, 5, 6, 7, 8, 9, 10, 11, 12, 13, 14, 15, 16, or greater. In some instances, the average DAR ratio includes whole number as well as fractions or decimal of a DAR ratio. In some instances, the average DAR ratio of the oligonucleotide molecule to antibody is about 1 or greater. In some instances, the average DAR ratio of the oligonucleotide molecule to antibody is about 2 or greater. In some instances, the average DAR ratio of the oligonucleotide molecule to antibody is about 3 or greater. In some instances, the average DAR ratio of the oligonucleotide molecule to antibody is about 4 or greater. In some instances, the average DAR ratio of the oligonucleotide molecule to antibody is about 5 or greater. In some instances, the average DAR ratio of the oligonucleotide molecule to antibody is about 6 or greater. In some instances, the average DAR ratio of the oligonucleotide molecule to antibody is about 7 or greater. In some instances, the average DAR ratio of the oligonucleotide molecule to antibody is about 8 or greater. In some instances, the average DAR ratio of the oligonucleotide molecule to antibody is about 9 or greater. In some instances, the average DAR ratio of the oligonucleotide molecule to antibody is about 10 or greater. In some instances, the average DAR ratio of the oligonucleotide molecule to antibody is about 11 or greater. In some instances, the average DAR ratio of the oligonucleotide molecule to antibody is about 12 or greater.

In some instances, the average DAR ratio of the oligonucleotide molecule to antibody is about 1, 2, 3, 4, 5, 6, 7, 8, 9, 10, 11, 12, 13, 14, 15, or 16. In some instances, the average DAR ratio of the oligonucleotide molecule to antibody is about 1. In some instances, the average DAR ratio of the oligonucleotide molecule to antibody is about 2. In some instances, the average DAR ratio of the oligonucleotide molecule to antibody is about 3. In some instances, the average DAR ratio of the oligonucleotide molecule to antibody is about 4. In some instances, the average DAR ratio of the oligonucleotide molecule to antibody is about 5. In some instances, the average DAR ratio of the oligonucleotide molecule to antibody is about 6. In some instances, the average DAR ratio of the oligonucleotide molecule to antibody is about 7. In some instances, the average DAR ratio of the oligonucleotide molecule to antibody is about 8. In some instances, the average DAR ratio of the oligonucleotide molecule to antibody is about 9. In some instances, the average DAR ratio of the oligonucleotide molecule to antibody is about 10. In some instances, the average DAR ratio of the oligonucleotide molecule to antibody is about 11. In some instances, the average DAR ratio of the oligonucleotide molecule to antibody is about 12. In some instances, the average DAR ratio of the oligonucleotide molecule to antibody is about 13. In some instances, the average DAR ratio of the oligonucleotide molecule to antibody is about 14. In some instances, the DAR ratio of the oligonucleotide molecule to antibody is about 15. In some instances, the average DAR ratio of the oligonucleotide molecule to antibody is about 16.

In some instances, the average DAR ratio of the oligonucleotide molecule to antibody is 1, 2, 3, 4, 5, 6, 7, 8, 9, 10, 11, 12, 13, 14, 15, or 16. In some instances, the average DAR ratio of the oligonucleotide molecule to antibody is 1. In some instances, the average DAR ratio of the oligonucleotide molecule to antibody is 2. In some instances, the average DAR ratio of the oligonucleotide molecule to antibody is 4. In some instances, the average DAR ratio of the oligonucleotide molecule to antibody is 6. In some instances, the average DAR ratio of the oligonucleotide molecule to antibody is 8. In some instances, the average DAR ratio of the oligonucleotide molecule to antibody is 12. In some instances, the average DAR ratio of the oligonucleotide molecule to antibody is 16.

In some instances, the average DAR ratio of the oligonucleotide molecule to antibody is in the range of 1.5-2.5, 2.5-3.5, 3.5-4.5, 4.5-5.5, 5.5-6.5, 6.5-7.5, 7.5-8.5, 8.5-9.5, 9.5-10.5, 10.5-11.5, 11.5-12.5, 12.5-13.5, 13.5-14.5, 14.5-15.5, 15.5-16.5, or 16.5-17.5. In some instances, the average DAR ratio of the oligonucleotide molecule to antibody is in the range of 1.5-2.5. In some instances, the average DAR ratio of the oligonucleotide molecule to antibody is in the range of 2.5-3.5. In some instances, the average DAR ratio of the oligonucleotide molecule to antibody is in the range of 3.5-4.5. In some instances, the average DAR ratio of the oligonucleotide molecule to antibody is in the range of 4.5-5.5. In some instances, the average DAR ratio of the oligonucleotide molecule to antibody is in the range of 5.5-6.5. In some instances, the average DAR ratio of the oligonucleotide molecule to antibody is in the range of 6.5-7.5. In some instances, the average DAR ratio of the oligonucleotide molecule to antibody is in the range of 7.5-8.5. In some instances, the average DAR ratio of the oligonucleotide molecule to antibody is in the range of 8.5-9.5. In some instances, the average DAR ratio of the oligonucleotide molecule to antibody is in the range of 9.5-10.5. In some instances, the average DAR ratio of the oligonucleotide molecule to antibody is in the range of 10.5-11.5. In some instances, the average DAR ratio of the oligonucleotide molecule to antibody is in the range of 11.5-12.5. In some instances, the average DAR ratio of the oligonucleotide molecule to antibody is in the range of 12.5-13.5. In some instances, the average DAR ratio of the oligonucleotide molecule to antibody is in the range of 13.5-14.5. In some instances, the average DAR ratio of the oligonucleotide molecule to antibody is in the range of 14.5-15.5. In some instances, the DAR ratio of the oligonucleotide molecule to antibody is in the range of 15.5-16.5. In some instances, the average DAR ratio of the oligonucleotide molecule to antibody is in the range of 16.5-17.5.

In some instances, a conjugate comprising an oligonucleotide molecule and an antibody has improved activity as compared to a conjugate comprising an oligonucleotide molecule without an antibody. In some instances, improved activity results in enhanced biologically relevant functions, e.g., improved stability, affinity, binding, functional activity, and efficacy in treatment or prevention of a disease state. In some instances, the disease state is a result of one or more mutated exons of a gene. In some instances, the conjugate comprising an oligonucleotide molecule and an antibody results in increased exon skipping of the one or more mutated exons as compared to the conjugate comprising an oligonucleotide molecule without an antibody. In some instances, exon skipping is increased by at least or about 5%, 10%, 20%, 25%, 30%, 40%, 50%, 60%, 70%, 80%, 90%, 95%, or more than 95% in the conjugate comprising oligonucleotide molecule and antibody as compared to the conjugate comprising oligonucleotide molecule without an antibody.

Thus, in some instances, an oligonucleotide molecule conjugate comprises an oligonucleotide molecule (e.g., PMO molecule) comprising or consisting of a sequence with at least 80%, at least 85%, at least 90%, at least 95%, at least 99%, or 100% sequence identity to a nucleic acid sequence selected from SEQ ID NOs: 100-119, and an anti-transferrin receptor antibody or antigen binding fragment thereof conjugated to the oligonucleotide such that the oligonucleotide molecule conjugate induces exon skipping of the pre-mRNA of the DMD gene. In some instances, an oligonucleotide molecule conjugate comprises an oligonucleotide molecule (e.g., PMO molecule) comprising or consisting of a sequence with at least 80%, at least 85%, at least 90%, at least 95%, at least 99%, or 100% sequence identity to a nucleic acid sequence selected from SEQ ID NOs: 100-109, and an anti-transferrin receptor antibody or antigen binding fragment thereof conjugated to the oligonucleotide such that the oligonucleotide molecule conjugate induces exon skipping of the pre-mRNA of the DMD gene.

In certain aspects, an oligonucleotide molecule conjugate comprises an anti-transferrin receptor antibody or antigen binding fragment thereof conjugated to an oligonucleotide molecule (e.g., PMO molecule) that hybridizes to a sequence of a target region of a pre-mRNA transcript of the DMD gene. In some instances, the oligonucleotide molecule (e.g., PMO molecule) comprises or consists of a sequence with at least 80%, at least 85%, at least 90%, at least 95%, at least 99%, or 100% sequence identity to a nucleic acid sequence selected from SEQ ID NOs: 100-109, and the oligonucleotide molecule (e.g., PMO molecule) has from about 24 to about 29 nucleotides, at least about 24 to about 28 nucleotides, at least about 24 to about 27 nucleotides, at least about 24 to about 26 nucleotides, at least about 24 to about 25 nucleotides, at least about 25 to about 29 nucleotides, at least about 25 to about 28 nucleotides, at least about 25 to about 27 nucleotides, at least about 25 to about 26 nucleotides, at least about 26 to about 29 nucleotides, at least about 26 to about 28 nucleotides, at least about 26 to about 27 nucleotides, at least about 27 to about 29 nucleotides, at least about 27 to about 28 nucleotides, at least about 28 to about 29 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, or at least 29 nucleotides in length, and anti-transferrin receptor antibody or antigen binding fragment thereof comprises a variable heavy chain (VH) region and a variable light chain (VL) region, wherein the VH region comprises an HCDR1 sequence comprising or consisting of SEQ ID NO: 17, an HCDR2 sequence comprising or consisting of SEQ ID NO: 20, and an HCDR3 sequence comprising or consisting of SEQ ID NO: 19; and the VL region comprises an LCDR1 sequence comprising or consisting of SEQ ID NO: 22, an LCDR2 sequence comprising or consisting of SEQ ID NO: 23, and an LCDR3 sequence comprising or consisting of SEQ ID NO: 24. In some instances, the anti-transferrin receptor antibody or antigen binding fragment thereof and the oligonucleotide molecule is conjugated via a linker. In some instances, the linker is 4-(N-maleimidomethyl) cyclohexane-1-amidate (SMCC). In some instances, the oligonucleotide molecule having a sense strand comprising or consisting of a nucleic acid sequence with at least 80%, at least 85%, at least 90%, at least 95%, at least 99%, or 100% sequence identity to a nucleic acid sequence selected from SEQ ID NOs: 100-119, and anti-transferrin receptor antibody or antigen binding fragment thereof comprises a variable heavy chain (VH) region and a variable light chain (VL) region, wherein the VH region comprises an HCDR1 sequence comprising or consisting of SEQ ID NO: 17, an HCDR2 sequence comprising or consisting of SEQ ID NO: 20, and an HCDR3 sequence comprising or consisting of SEQ ID NO: 19; and the VL region comprises an LCDR1 sequence comprising or consisting of SEQ ID NO: 22, an LCDR2 sequence comprising or consisting of SEQ ID NO: 23, and an LCDR3 sequence comprising or consisting of SEQ ID NO: 24, and the anti-transferrin receptor antibody or antigen binding fragment thereof and the oligonucleotide molecule is conjugated via a linker comprising 4-(N-maleimidomethyl) cyclohexane-1-amidate (SMCC).

In certain aspects, an oligonucleotide molecule conjugate comprises an anti-transferrin receptor antibody or antigen binding fragment thereof conjugated to an oligonucleotide molecule (e.g., PMO molecule) that hybridizes to a sequence of a target region of a pre-mRNA transcript of the DMD gene. In some instances, the oligonucleotide molecule (e.g., PMO molecule) comprising or consisting of a sequence with at least 80%, at least 85%, at least 90%, at least 95%, at least 99%, or 100% sequence identity to a nucleic acid sequence selected from SEQ ID NOs: 100-109, and the oligonucleotide molecule (e.g., PMO molecule) has from about 24 to about 29 nucleotides, at least about 24 to about 28 nucleotides, at least about 24 to about 27 nucleotides, at least about 24 to about 26 nucleotides, at least about 24 to about 25 nucleotides, at least about 25 to about 29 nucleotides, at least about 25 to about 28 nucleotides, at least about 25 to about 27 nucleotides, at least about 25 to about 26 nucleotides, at least about 26 to about 29 nucleotides, at least about 26 to about 28 nucleotides, at least about 26 to about 27 nucleotides, at least about 27 to about 29 nucleotides, at least about 27 to about 28 nucleotides, at least about 28 to about 29 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, or at least 29 nucleotides in length, and the anti-transferrin receptor antibody or antigen binding fragment thereof comprises a variable heavy chain (VH) region and a variable light chain (VL) region, wherein the VH region comprises or consisting of a nucleic acid sequence with at least 80%, 85%, 90%, 95%, 99%, or 100% sequence identity to SEQ ID NO: 30, and wherein the VL region comprises or consisting of a nucleic acid sequence with at least 80%, 85%, 90%, 95%, 99%, or 100% sequence identity to SEQ ID NO: 34. In some instances, the anti-transferrin receptor antibody or antigen binding fragment thereof and the oligonucleotide molecule is conjugated via a linker. In some instances, the linker is maleimide linker. In some instances.the oligonucleotide molecule comprises or consists of a nucleic acid sequence with at least 80%, at least 85%, at least 90%, at least 95%, at least 99%, or 100% sequence identity to a nucleic acid sequence selected from SEQ ID NOs: 100-119 and the anti-transferrin receptor antibody or antigen binding fragment thereof comprises a variable heavy chain (VH) region and a variable light chain (VL) region, wherein the VH region comprises or consisting of a nucleic acid sequence with at least 80%, 85%, 90%, 95%, 99%, or 100% sequence identity to SEQ ID NO: 30, and wherein the VL region comprises or consisting of a nucleic acid sequence with at least 80%, 85%, 90%, 95%, 99%, or 100% sequence identity to SEQ ID NO: 34, and the anti-transferrin receptor antibody or antigen binding fragment thereof and the oligonucleotide molecule is conjugated via a maleimide linker.

In certain aspects, an oligonucleotide molecule conjugate comprises an anti-transferrin receptor antibody or antigen binding fragment thereof conjugated to an oligonucleotide molecule (e.g., PMO molecule) that hybridizes to a sequence of a target region of a pre-mRNA transcript of the DMD gene, and the oligonucleotide molecule comprising or consisting of a nucleic acid sequence with at least 80%, at least 85%, at least 90%, at least 95%, at least 99%, or 100% sequence identity to a nucleic acid sequence selected from SEQ ID NOs: 100-119, and comprises at least three, four, five, or six consecutive 2′-O-methyl modified nucleotides at the 5′-end and at least two, at least three 2′-F modified nucleotides, and the anti-transferrin receptor antibody or antigen binding fragment thereof comprises a variable heavy chain (VH) region and a variable light chain (VL) region, wherein the VH region comprises or consisting of a sequence with at least 80%, 85%, 90%, 95%, 99%, or 100% sequence identity to SEQ ID NO: 30, and wherein the VL region comprises or consisting of a sequence with at least 80%, 85%, 90%, 95%, 99%, or 100% sequence identity to SEQ ID NO: 34, and the anti-transferrin receptor antibody or antigen binding fragment thereof and the oligonucleotide molecule is conjugated via a maleimide linker.

In certain aspects, an oligonucleotide molecule conjugate comprises an anti-transferrin receptor antibody or antigen binding fragment thereof conjugated to an oligonucleotide molecule (e.g., PMO molecule) that hybridizes to a sequence of a target region of a pre-mRNA transcript of the DMD gene, and the oligonucleotide molecule comprising or consisting of a nucleic acid sequence with at least 80%, at least 85%, at least 90%, at least 95%, at least 99%, or 100% sequence identity to a nucleic acid sequence selected from SEQ ID NOs: 100-119, and comprises at least two, at least three, at least four, or at least five consecutive 2′-O-methyl modified nucleotide at the 3′-end of the oligonucleotide molecule, and comprises at least one, at least two, at least three, at least four 2′-F modified nucleotides, and the anti-transferrin receptor antibody or antigen binding fragment thereof comprises a variable heavy chain (VH) region and a variable light chain (VL) region, wherein the VH region comprises an HCDR1 sequence comprising or consisting of SEQ ID NO: 17, an HCDR2 sequence comprising or consisting of SEQ ID NO: 20, and an HCDR3 sequence comprising or consisting of SEQ ID NO: 19; and the VL region comprises an LCDR1 sequence comprising or consisting of SEQ ID NO: 22, an LCDR2 sequence comprising or consisting of SEQ ID NO: 23, and an LCDR3 sequence comprising or consisting of SEQ ID NO: 24, and the anti-transferrin receptor antibody or antigen binding fragment thereof and the oligonucleotide molecule is conjugated via a maleimide linker.

In certain aspects, an oligonucleotide molecule conjugate comprises an anti-transferrin receptor antibody or antigen binding fragment thereof conjugated to an oligonucleotide molecule (e.g., PMO molecule) that hybridizes to a sequence of a target region of a pre-mRNA transcript of the DMD gene, and the oligonucleotide molecule comprises or consists of a nucleic acid sequence with at least 80%, at least 85%, at least 90%, at least 95%, at least 99%, or 100% sequence identity to a nucleic acid sequence selected from SEQ ID NOs: 100-119, comprises one or more 2′-O-methyl modified nucleotides at the 5′-end and/or at the 3′-end of the oligonucleotide molecule, and the anti-transferrin receptor antibody or antigen binding fragment thereof comprises a variable heavy chain (VH) region and a variable light chain (VL) region, and the VH region comprises an HCDR1 sequence comprising or consisting of SEQ ID NO: 17, an HCDR2 sequence comprising or consisting of SEQ ID NO: 18, and an HCDR3 sequence comprising or consisting of SEQ ID NO: 19; and the VL region comprises an LCDR1 sequence comprising or consisting of SEQ ID NO: 22, an LCDR2 sequence comprising or consisting of SEQ ID NO: 3, and an LCDR3 sequence comprising or consisting of SEQ ID NO: 24, and the anti-transferrin receptor antibody or antigen binding fragment thereof and the oligonucleotide molecule is conjugated via a maleimide linker.

In certain aspects, an oligonucleotide molecule conjugate comprises an anti-transferrin receptor antibody or antigen binding fragment thereof conjugated to an oligonucleotide molecule (e.g., PMO molecule) that hybridizes to a target sequence of a pre-mRNA transcript of the DMD gene, and the oligonucleotide molecule comprising or consisting of a nucleic acid sequence with at least 80%, at least 85%, at least 90%, at least 95%, at least 99%, or 100% sequence identity to a nucleic acid sequence selected from SEQ ID NOs: 100-119, and the anti-transferrin receptor antibody or antigen binding fragment thereof comprises a variable heavy chain (VH) region and a variable light chain (VL) region, and the VH region comprises or consists of a sequence with at least 80%, at least 85%, at least 90%, at least 95%, at least 99%, or 100% sequence identity to one of SEQ ID NOs: 29-33, and wherein the VL region comprises or consists of a sequence with at least 80%, at least 85%, at least 90%, at least 95%, at least 99%, or 100% sequence identity to one of SEQ ID NOs: 34-38, and the anti-transferrin receptor antibody or antigen binding fragment thereof and the oligonucleotide molecule is conjugated via a 6-Amino-1-hexanol linker.

In some aspects, the average number of oligonucleotide (e.g., PMO) molecules conjugated to an antibody forms an average ratio. In some instances, the average ratio is referred to as an average DAR (drug-to-antibody) ratio, in which the drug as referred to herein is the oligonucleotide (e.g., PMO) molecule. In some instances, the average DAR ratio of the oligonucleotide (e.g., PMO) molecule to antibody is in the range of 1.0-2.0, 2.0-3.0, 3.0-4.0, 4.0-5.0, 5.0-6.0, 6.0-7.0, 7.0-8.0, 8.0-9.0, 9.0-10.0, 10.0-11.0, 11.0-12.0, 12.0-13.0, 13.0-14.0, 14.0-15.0, 15.0-16.0, or 16.0-17.0. In some instances, the average DAR ratio of the oligonucleotide (e.g., PMO) molecule to antibody is in the range of 1.0-2.0. In some instances, the average DAR ratio of the oligonucleotide (e.g., PMO) molecule to antibody is in the range of 2.0-3.0. In some instances, the average DAR ratio of the oligonucleotide (e.g., PMO) molecule to antibody is in the range of 3.0-4.0. In some instances, the average DAR ratio of oligonucleotide (e.g., PMO) molecule to antibody is in the range of 4.0-5.0. In some instances, the average DAR ratio of the oligonucleotide (e.g., PMO) molecules to antibody is in the range of 5.0-6.0. In some instances, the average DAR ratio of the oligonucleotide (e.g., PMO) molecule to antibody is in the range of 6.0-7.0. In some instances, the average DAR ratio of the oligonucleotide (e.g., PMO) molecule to antibody is in the range of 7.0-8.0.

In some aspects, the average number of oligonucleotide (e.g., PMO) molecules conjugated to an antibody forms an average ratio. In some instances, the average ratio is referred to as an average DAR (drug-to-antibody) ratio, in which the drug as referred to herein is the oligonucleotide (e.g., PMO) molecule. In some instances, the average DAR ratio of the oligonucleotide (e.g., PMO) molecule to antibody is in the range of 1.5-2.5, 2.5-3.5, 3.5-4.5, 4.5-5.5, 5.5-6.5, 6.5-7.5, 7.5-8.5, 8.5-9.5, 9.5-10.5, 10.5-11.5, 11.5-12.5, 12.5-13.5, 13.5-14.5, 14.5-15.5, 15.5-16.5, or 16.5-17.5. In some instances, the average DAR ratio of the oligonucleotide (e.g., PMO) molecule to antibody is in the range of 1.5-2.5. In some instances, the average DAR ratio of the oligonucleotide (e.g., PMO) molecule to antibody is in the range of 2.5-3.5. In some instances, the average DAR ratio of the oligonucleotide (e.g., PMO) molecule to antibody is in the range of 3.5-4.5. In some instances, the average DAR ratio of oligonucleotide (e.g., PMO) molecule to antibody is in the range of 4.5-5.5. In some instances, the average DAR ratio of the oligonucleotide (e.g., PMO) molecules to antibody is in the range of 5.5-6.5. In some instances, the average DAR ratio of the oligonucleotide (e.g., PMO) molecule to antibody is in the range of 6.5-7.5. In some instances, the average DAR ratio of the oligonucleotide (e.g., PMO) molecule to antibody is in the range of 7.5-8.5.

### Conjugation Chemistry

In some aspects, the oligonucleotide molecule (e.g., PMO) disclosed herein is conjugated to an antibody (e.g., the antibody disclosed herein). In some instances, the antibody comprises amino acids, peptides, polypeptides, proteins, antibodies, antigens, toxins, hormones, lipids, nucleotides, nucleosides, sugars, carbohydrates, polymers such as polyethylene glycol and polypropylene glycol, as well as analogs or derivatives of all of these classes of substances. Additional examples of antibody also include steroids, such as cholesterol, phospholipids, di- and triacylglycerols, fatty acids, hydrocarbons (e.g., saturated, unsaturated, or contains substitutions), enzyme substrates, biotin, digoxigenin, and polysaccharides. In some instances, the oligonucleotide molecule is further conjugated to a polymer, and optionally an endosomolytic moiety.

In some aspects, the oligonucleotide molecule is conjugated to the antibody by a chemical ligation process. In some instances, the oligonucleotide molecule is conjugated to the antibody by a native ligation. In some instances, the conjugation is as described in: Dawson, et al. “Synthesis of proteins by native chemical ligation,” Science 1994, 266, 776-779; Dawson, et al. “Modulation of Reactivity in Native Chemical Ligation through the Use of Thiol Additives,” J. Am. Chem. Soc. 1997, 119, 4325-4329; Hackeng, et al. “Protein synthesis by native chemical ligation: Expanded scope by using straightforward methodology.,” Proc. Natl. Acad. Sci. USA 1999, 96, 10068-10073; or Wu, et al. “Building complex glycopeptides: Development of a cysteine-free native chemical ligation protocol,” Angew. Chem. Int. Ed. 2006, 45, 4116-4125. In some instances, the conjugation is as described in U.S. Pat. No. 8,936,910. In some aspects, the oligonucleotide molecule is conjugated to the antibody either site-specifically or non-specifically via native ligation chemistry.

In some instances, the oligonucleotide molecule is conjugated to the antibody by a site-directed method utilizing a “traceless” coupling technology (Philochem). In some instances, the “traceless” coupling technology utilizes an N-terminal 1,2-aminothiol group on the antibody which is then conjugated with an oligonucleotide molecule containing an aldehyde group. (see Casi et al., “Site-specific traceless coupling of potent cytotoxic drugs to recombinant antibodies for pharmacodelivery,” JACS 134(13): 5887-5892 (2012))

In some instances, the oligonucleotide molecule is conjugated to the antibody by a site-directed method utilizing an unnatural amino acid incorporated into the antibody. In some instances, the unnatural amino acid comprises p-acetylphenylalanine (pAcPhe). In some instances, the keto group of pAcPhe is selectively coupled to an alkoxy-amine derivatived conjugating moiety to form an oxime bond. (see Axup et al., “Synthesis of site-specific antibody-drug conjugates using unnatural amino acids,” PNAS 109(40): 16101-16106 (2012)).

In some instances, the oligonucleotide molecule is conjugated to the antibody by a site-directed method utilizing an enzyme-catalyzed process. In some instances, the site-directed method utilizes SMARTag™ technology (Redwood). In some instances, the SMARTag™ technology comprises generation of a formylglycine (FGly) residue from cysteine by formylglycine-generating enzyme (FGE) through an oxidation process under the presence of an aldehyde tag and the subsequent conjugation of FGly to an alkylhydraine-functionalized oligonucleotide molecule via hydrazino-Pictet-Spengler (HIPS) ligation. (see Wu et al., “Site-specific chemical modification of recombinant proteins produced in mammalian cells by using the genetically encoded aldehyde tag,” PNAS 106(9): 3000-3005 (2009); Agarwal, et al., “A Pictet-Spengler ligation for protein chemical modification,” PNAS 110(1): 46-51 (2013))

In some instances, the enzyme-catalyzed process comprises microbial transglutaminase (mTG). In some cases, the oligonucleotide molecule is conjugated to the antibody utilizing a microbial transglutaminze catalyzed process. In some instances, mTG catalyzes the formation of a covalent bond between the amide side chain of a glutamine within the recognition sequence and a primary amine of a functionalized oligonucleotide molecule. In some instances, mTG is produced from Streptomyces mobarensis. (see Strop et al., “Location matters: site of conjugation modulates stability and pharmacokinetics of antibody drug conjugates,” Chemistry and Biology 20(2) 161-167 (2013))

In some instances, the oligonucleotide molecule is conjugated to the antibody by a method as described in PCT Publication No. WO2014/140317, which utilizes a sequence-specific transpeptidase. In some instances, the oligonucleotide molecule is conjugated to the antibody by a method as described in U.S. Patent Publication Nos. 2015/0105539 and 2015/0105540.

### Linkers

In some aspects, a linker described herein is a cleavable linker or a non-cleavable linker. In some instances, the linker is a cleavable linker. In other instances, the linker is a non-cleavable linker.

In some cases, the linker is a non-polymeric linker. A non-polymeric linker refers to a linker that does not contain a repeating unit of monomers generated by a polymerization process. Exemplary non-polymeric linkers include, but are not limited to, C1-C6 alkyl group (e.g., a C5, C4, C3, C2, or C1 alkyl group), homobifunctional cross linkers, heterobifunctional cross linkers, peptide linkers, traceless linkers, self-immolative linkers, maleimide-based linkers, or combinations thereof. In some cases, the non-polymeric linker comprises a C1-C6 alkyl group (e.g., a C5, C4, C3, C2, or C1 alkyl group), a homobifunctional cross linker, a heterobifunctional cross linker, a peptide linker, a traceless linker, a self-immolative linker, a maleimide-based linker, or a combination thereof. In additional cases, the non-polymeric linker does not comprise more than two of the same type of linkers, e.g., more than two homobifunctional cross linkers, or more than two peptide linkers. In further cases, the non-polymeric linker optionally comprises one or more reactive functional groups.

In some instances, the non-polymeric linker does not encompass a polymer that is described above. In some instances, the non-polymeric linker does not encompass a polymer encompassed by the polymer moiety C. In some cases, the non-polymeric linker does not encompass a polyalkylene oxide (e.g., PEG). In some cases, the non-polymeric linker does not encompass a PEG.

In some instances, the linker comprises a homobifunctional linker. Exemplary homobifunctional linkers include, but are not limited to, Lomant's reagent dithiobis (succinimidylpropionate) DSP, 3′3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[B-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), or N,N′-hexamethylene-bis(iodoacetamide).

In some aspects, the linker comprises a heterobifunctional linker. Exemplary heterobifunctional linker include, but are not limited to, amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-α-methyl-α-(2-pyridyldithio)toluene (sMPT), sulfosuccinimidyl-6-[α-methyl-α-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N-(γ-maleimidobutyryloxy)succinimide ester (GMBs), N-(γ-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M2C2H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(ρ-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl)1,3′-dithiopropionate (sADP), N-sulfosuccinimidyl(4-azidophenyl)-1,3′-dithiopropionate (sulfo-sADP), sulfosuccinimidyl 4-(ρ-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), ρ-nitrophenyl diazopyruvate (pNPDP), ρ-nitrophenyl-2-diazo-3,3,3-trifluoropropionate (PNP-DTP), sulfhydryl-reactive and photoreactive cross-linkers such as 1-(ρ-Azidosalicylamido)-4-(iodoacetamido)butane (AsIB), N-[4-(ρ-azidosalicylamido)butyl]-3′-(2′-pyridyldithio)propionamide (APDP), benzophenone-4-iodoacetamide, benzophenone-4-maleimide carbonyl-reactive and photoreactive cross-linkers such as ρ-azidobenzoyl hydrazide (ABH), carboxylate-reactive and photoreactive cross-linkers such as 4-(ρ-azidosalicylamido)butylamine (AsBA), and arginine-reactive and photoreactive cross-linkers such as ρ-azidophenyl glyoxal (APG).

In some instances, the linker comprises a reactive functional group. In some cases, the reactive functional group comprises a nucleophilic group that is reactive to an electrophilic group present on an antibody. Exemplary electrophilic groups include carbonyl groups-such as aldehyde, ketone, carboxylic acid, ester, amide, enone, acyl halide or acid anhydride. In some aspects, the reactive functional group is aldehyde. Exemplary nucleophilic groups include hydrazide, oxime, amino, hydrazine, thiosemicarbazone, hydrazine carboxylate, and arylhydrazide.

In some aspects, the linker comprises a maleimide group. In some instances, the maleimide group is also referred to as a maleimide spacer. In some instances, the maleimide group further encompasses a caproic acid, forming maleimidocaproyl (mc). In some cases, the linker comprises maleimidocaproyl (mc). In some cases, the linker is maleimidocaproyl (mc). In other instances, the maleimide group comprises a maleimidomethyl group, such as succinimidyl-4-(N-maleimidomethyl)cyclohexane-1-carboxylate (sMCC) or sulfosuccinimidyl-4-(N-maleimidomethyl)cyclohexane-1-carboxylate (sulfo-sMCC) described above.

In some aspects, the maleimide group is a self-stabilizing maleimide. In some instances, the self-stabilizing maleimide utilizes diaminopropionic acid (DPR) to incorporate a basic amino group adjacent to the maleimide to provide intramolecular catalysis of tiosuccinimide ring hydrolysis, thereby eliminating maleimide from undergoing an elimination reaction through a retro-Michael reaction. In some instances, the self-stabilizing maleimide is a maleimide group described in Lyon, et al., “Self-hydrolyzing maleimides improve the stability and pharmacological properties of antibody-drug conjugates,” Nat. Biotechnol. 32(10):1059-1062 (2014). In some instances, the linker comprises a self-stabilizing maleimide. In some instances, the linker is a self-stabilizing maleimide.

In some aspects, the linker comprises a peptide moiety. In some instances, the peptide moiety comprises at least 2, 3, 4, 5, or 6 more amino acid residues. In some instances, the peptide moiety comprises at most 2, 3, 4, 5, 6, 7, or 8 amino acid residues. In some instances, the peptide moiety comprises about 2, about 3, about 4, about 5, or about 6 amino acid residues. In some instances, the peptide moiety is a cleavable peptide moiety (e.g., either enzymatically or chemically). In some instances, the peptide moiety is a non-cleavable peptide moiety. In some instances, the peptide moiety comprises Val-Cit (valine-citrulline), Gly-Gly-Phe-Gly (SEQ ID NO: 300), Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu (SEQ ID NO: 301), or Gly-Phe-Leu-Gly (SEQ ID NO: 302). In some instances, the linker comprises a peptide moiety such as: Val-Cit (valine-citrulline), Gly-Gly-Phe-Gly (SEQ ID NO:300), Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu (SEQ ID NO: 301), or Gly-Phe-Leu-Gly (SEQ ID NO:302). In some cases, the linker comprises Val-Cit. In some cases, the linker is Val-Cit.

In some aspects, the linker comprises a benzoic acid group, or its derivatives thereof. In some instances, the benzoic acid group or its derivatives thereof comprise paraaminobenzoic acid (PABA). In some instances, the benzoic acid group or its derivatives thereof comprise gamma-aminobutyric acid (GABA).

In some aspects, the linker comprises one or more of a maleimide group, a peptide moiety, and/or a benzoic acid group, in any combination. In some aspects, the linker comprises a combination of a maleimide group, a peptide moiety, and/or a benzoic acid group. In some instances, the maleimide group is maleimidocaproyl (mc). In some instances, the peptide group is val-cit. In some instances, the benzoic acid group is PABA. In some instances, the linker comprises a mc-val-cit group. In some cases, the linker comprises a val-cit-PABA group. In additional cases, the linker comprises a mc-val-cit-PABA group.

In some aspects, the linker is a self-immolative linker or a self-elimination linker. In some cases, the linker is a self-immolative linker. In other cases, the linker is a self-elimination linker (e.g., a cyclization self-elimination linker). In some instances, the linker comprises a linker described in U.S. Pat. No. 9,089,614 or PCT Publication No. WO2015038426.

In some aspects, the linker is a dendritic type linker. In some instances, the dendritic type linker comprises a branching, multifunctional linker moiety. In some instances, the dendritic type linker is used to increase the molar ratio of polynucleotide to the antibody. In some instances, the dendritic type linker comprises PAMAM dendrimers.

In some aspects, the linker is a traceless linker or a linker in which after cleavage does not leave behind a linker moiety (e.g., an atom or a linker group) to a binding moiety (e.g., an antibody), a polynucleotide, a polymer, or an endosomolytic moiety. Exemplary traceless linkers include, but are not limited to, germanium linkers, silicium linkers, sulfur linkers, selenium linkers, nitrogen linkers, phosphorus linkers, boron linkers, chromium linkers, or phenylhydrazide linkers. In some cases, the linker is a traceless aryl-triazene linker as described in Hejesen, et al., “A traceless aryl-triazene linker for DNA-directed chemistry,” Org Biomol Chem 11(15): 2493-2497 (2013). In some instances, the linker is a traceless linker described in Blaney, et al., “Traceless solid-phase organic synthesis,” Chem. Rev. 102: 2607-2024 (2002). In some instances, a linker is a traceless linker as described in U.S. Pat. No. 6,821,783.

In some instances, the linker is a linker described in U.S. Pat. Nos. 6,884,869; 7,498,298; 8,288,352; 8,609,105; or 8,697,688; U.S. Patent Publication Nos. 2014/0127239; 2013/028919; 2014/286970; 2013/0309256; 2015/037360; or 2014/0294851; or PCT Publication Nos. WO2015057699; WO2014080251; WO2014197854; WO2014145090; or WO2014177042.

In some instances, the linker is a C1-C6 alkyl group. In some cases, the linker is a C1-C6 alkyl group, such as for example, a C5, C4, C3, C2, or C1 alkyl group. In some cases, the C1-C6 alkyl group is an unsubstituted C1-C6 alkyl group. As used in the context of a linker, and in particular in the context of the linker, alkyl means a saturated straight or branched hydrocarbon radical containing up to six carbon atoms. In some instances, the linker is a non-polymeric linker. In some instances, the linker includes a homobifunctional linker or a heterobifunctional linker described supra. In some cases, the linker includes a heterobifunctional linker. In some cases, the linker includes or comprises sMCC. In other instances, the linker includes a heterobifunctional linker optionally conjugated to a C1-C6 alkyl group. In other instances, the linker includes sMCC optionally conjugated to a C1-C6 alkyl group. In additional instances, the linker does not include a homobifunctional linker or a heterobifunctional linker described supra.

### Pharmaceutical Formulation

In some aspects, the pharmaceutical formulations described herein are administered to a subject by multiple administration routes, including but not limited to, parenteral (e.g., intravenous, subcutaneous, intramuscular), oral, intranasal, buccal, rectal, or transdermal administration routes. In some instances, the pharmaceutical composition described herein is formulated for parenteral (e.g., intravenous, subcutaneous, intramuscular, intra-arterial, intraperitoneal, intrathecal, intracerebral, intracerebroventricular, or intracranial) administration. In other instances, the pharmaceutical composition described herein is formulated for oral administration. In still other instances, the pharmaceutical composition described herein is formulated for intranasal administration.

In some aspects, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some instances, the pharmaceutical formulation includes multiparticulate formulations. In some instances, the pharmaceutical formulation includes nanoparticle formulations. In some instances, nanoparticles comprise cMAP, cyclodextrin, or lipids. In some cases, nanoparticles comprise solid lipid nanoparticles, polymeric nanoparticles, self-emulsifying nanoparticles, liposomes, microemulsions, or micellar solutions. Additional exemplary nanoparticles include, but are not limited to, paramagnetic nanoparticles, superparamagnetic nanoparticles, metal nanoparticles, fullerene-like materials, inorganic nanotubes, dendrimers (such as with covalently attached metal chelates), nanofibers, nanohorns, nano-onions, nanorods, nanoropes and quantum dots. In some instances, a nanoparticle is a metal nanoparticle, e.g., a nanoparticle of scandium, titanium, vanadium, chromium, manganese, iron, cobalt, nickel, copper, zinc, yttrium, zirconium, niobium, molybdenum, ruthenium, rhodium, palladium, silver, cadmium, hafnium, tantalum, tungsten, rhenium, osmium, iridium, platinum, gold, gadolinium, aluminum, gallium, indium, tin, thallium, lead, bismuth, magnesium, calcium, strontium, barium, lithium, sodium, potassium, boron, silicon, phosphorus, germanium, arsenic, antimony, and combinations, alloys or oxides thereof.

In some instances, a nanoparticle includes a core or a core and a shell, as in a core-shell nanoparticle.

In some instances, a nanoparticle is further coated with molecules for attachment of functional elements (e.g., with one or more of a polynucleic acid molecule or binding moiety (e.g., antibody described herein)). In some instances, a coating comprises chondroitin sulfate, dextran sulfate, carboxymethyl dextran, alginic acid, pectin, carragheenan, fucoidan, agaropectin, porphyran, karaya gum, gellan gum, xanthan gum, hyaluronic acids, glucosamine, galactosamine, chitin (or chitosan), polyglutamic acid, polyaspartic acid, lysozyme, cytochrome C, ribonuclease, trypsinogen, chymotrypsinogen, a-chymotrypsin, polylysine, polyarginine, histone, protamine, ovalbumin or dextrin or cyclodextrin. In some instances, a nanoparticle comprises a graphene-coated nanoparticle.

In some cases, a nanoparticle has at least one dimension of less than about 500 nm, 400 nm, 300 nm, 200 nm, or 100 nm.

In some instances, the nanoparticle formulation comprises paramagnetic nanoparticles, superparamagnetic nanoparticles, metal nanoparticles, fullerene-like materials, inorganic nanotubes, dendrimers (such as with covalently attached metal chelates), nanofibers, nanohorns, nano-onions, nanorods, nanoropes or quantum dots. In some instances, a polynucleic acid molecule or a binding moiety (e.g., antibody) described herein is conjugated either directly or indirectly to the nanoparticle. In some instances, at least 1, 5, 10, 15, 20, 30, 40, 50, 60, 70, 80, 90, 100 or more polynucleic acid molecules or binding moieties described herein are conjugated either directly or indirectly to a nanoparticle.

In some aspects, the pharmaceutical formulation comprise a delivery vector, e.g., a recombinant vector, the delivery of the polynucleic acid molecule into cells. In some instances, the recombinant vector is DNA plasmid. In other instances, the recombinant vector is a viral vector. Exemplary viral vectors include vectors derived from adeno-associated virus, retrovirus, adenovirus, or alphavirus. In some instances, the recombinant vectors capable of expressing the polynucleic acid molecules provide stable expression in target cells. In additional instances, viral vectors are used that provide for transient expression of polynucleic acid molecules.

In some aspects, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

### Therapeutic Regimens

In some aspects, the pharmaceutical compositions described herein are administered for therapeutic applications. In some aspects, the pharmaceutical composition is administered once per day, twice per day, three times per day or more. The pharmaceutical composition is administered daily, every day, every alternate day, five days a week, once a week, every other week, two weeks per month, three weeks per month, once a month, twice a month, three times per month, or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In some aspects, one or more pharmaceutical compositions are administered simultaneously, sequentially, or at an interval period of time. In some aspects, one or more pharmaceutical compositions are administered simultaneously. In some cases, one or more pharmaceutical compositions are administered sequentially. In additional cases, one or more pharmaceutical compositions are administered at an interval period of time (e.g., the first administration of a first pharmaceutical composition is on day one followed by an interval of at least 1, 2, 3, 4, 5, or more days prior to the administration of at least a second pharmaceutical composition).

In some aspects, two or more different pharmaceutical compositions are coadministered. In some instances, the two or more different pharmaceutical compositions are coadministered simultaneously. In some cases, the two or more different pharmaceutical compositions are coadministered sequentially without a gap of time between administrations. In other cases, the two or more different pharmaceutical compositions are coadministered sequentially with a gap of about 0.5 hours, 1 hour, 2 hours, 3 hours, 12 hours, 1 day, 2 days, or more between administrations.

In the case wherein the patient's status does improve, upon the doctor's discretion the administration of the composition is given continuously; alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained.

In some aspects, the amount of a given agent that corresponds to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some aspects, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in humans. The dosage of such compounds lies preferably within a range of circulating concentrations that include the ED50 with minimal toxicity. The dosage varies within this range depending upon the dosage form employed and the route of administration utilized.

### Kits/Article of Manufacture

Disclosed herein, in certain aspects, are kits and articles of manufacture for use with one or more of the compositions and methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include target nucleic acid molecule described herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain aspects, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### Methods and Uses

In some aspects, a polynucleic acid molecule (oligonucleotide, e.g., PMO, ASO, etc.) or a pharmaceutical composition comprising the polynucleic acid molecule described herein is used for the treatment of a disease or disorder characterized with a defective mRNA. In some aspects, a polynucleic acid molecule (oligonucleotide, e.g., PMO, ASO, etc.) or a pharmaceutical composition comprising the polynucleic acid molecule described herein is used for the treatment of disease or disorder by inducing an insertion, deletion, duplication, or alteration in an incorrectly spliced mRNA transcript to induce exon skipping or exon inclusion.

A large percentage of human protein-coding genes are alternatively spliced. In some instances, a mutation results in improperly spliced or partially spliced mRNA. For example, a mutation can be in at least one of a splice site in a protein coding gene, a silencer or enhancer sequence, exonic sequences, or intronic sequences. In some instances, a mutation results in gene dysfunction. In some instances, a mutation results in a disease or disorder.

Improperly spliced or partially spliced mRNA in some instances causes a neuromuscular disease or disorder. Exemplary neuromuscular diseases include muscular dystrophy such as Duchenne muscular dystrophy, Becker muscular dystrophy, facioscapulohumeral muscular dystrophy, congenital muscular dystrophy, or myotonic dystrophy. In some instances, muscular dystrophy is genetic. In some instances, muscular dystrophy is caused by a spontaneous mutation. Becker muscular dystrophy and Duchenne muscular dystrophy have been shown to involve mutations in the DMD gene, which encodes the protein dystrophin.

In some instances, improperly spliced or partially spliced mRNA causes Duchenne muscular dystrophy. Duchenne muscular dystrophy results in severe muscle weakness and is caused by mutations in the DMD gene that abolishes the production of functional dystrophin. In some instances, Duchenne muscular dystrophy is a result of a mutation in exon 45 in the DMD gene. In some instances, multiple exons are mutated/deleted. For example, mutations of exons 45 and 45 are common in Duchenne muscular dystrophy patients. In some instances, Duchenne muscular dystrophy is a result of mutation of exon 45. In some instances, Duchenne muscular dystrophy is a result of mutation of exon 45 and deletion of exon 45.

In some instances, a polynucleic acid molecule (oligonucleotide, e.g., PMO, ASO, etc.) or a pharmaceutical composition comprising the polynucleic acid molecule described herein is used for the treatment of muscular dystrophy. In some instances, a polynucleic acid molecule (oligonucleotide, e.g., PMO, ASO, etc.) or a pharmaceutical composition comprising the polynucleic acid molecule described herein is used for the treatment of muscular dystrophy that results from a mutation of the dystrophin gene that is amenable to exon 45 skipping. In some instances, a polynucleic acid molecule (oligonucleotide, e.g., PMO, ASO, etc.) or a pharmaceutical composition comprising the polynucleic acid molecule described herein is used for the treatment of muscular dystrophy that results from a deletion leading to a premature stop codon in exon 45. In some instances, a polynucleic acid molecule (oligonucleotide, e.g., PMO, ASO, etc.) or a pharmaceutical composition comprising the polynucleic acid molecule described herein is used for the treatment of muscular dystrophy that results from a deletion of e.g., exons 46-55, exons 18-44, or exon 44.

In some instances, a polynucleic acid-binding moiety (e.g., antibody) conjugate or a pharmaceutical composition comprising the polynucleic acid-binding moiety (e.g., antibody) conjugate as described herein is used for the treatment of muscular dystrophy. In some instances, a polynucleic acid-binding moiety (e.g., antibody) conjugate or a pharmaceutical composition comprising the polynucleic acid-binding moiety (e.g., antibody) conjugate as described herein is used for the treatment of Duchenne muscular dystrophy, Becker muscular dystrophy, facioscapulohumeral muscular dystrophy, congenital muscular dystrophy, or myotonic dystrophy. In some instances, a polynucleic acid-binding moiety (e.g., antibody) conjugate or a pharmaceutical composition comprising the polynucleic acid-binding moiety (e.g., antibody) conjugate as described herein is used for the treatment of Duchenne muscular dystrophy. In some instances, an oligonucleotide (e.g., PMO)-binding moiety (e.g., antibody) conjugate or a pharmaceutical composition comprising the oligonucleotide (e.g., PMO)-binding moiety (e.g., antibody) conjugate as described herein is used to induce exon 45 skipping for the treatment of muscular dystrophy. In some instances, an oligonucleotide (e.g., PMO)-binding moiety (e.g., antibody) conjugate or a pharmaceutical composition comprising the oligonucleotide (e.g., PMO)-binding moiety (e.g., antibody) conjugate as described herein is used to induce exon 45 skipping for the treatment of Duchenne muscular dystrophy or Becker muscular dystrophy. In some instances, an oligonucleotide (e.g., PMO)-binding moiety (e.g., antibody) conjugate or a pharmaceutical composition comprising the oligonucleotide (e.g., PMO)-binding moiety (e.g., antibody) conjugate as described herein is used to induce exon 45 skipping for the treatment of Duchenne muscular dystrophy.

In some aspects, provided herein a method of inducing exon 45 skipping in a subject in need thereof comprising administering to the subject the oligonucleotide molecule (e.g., PMO) disclosed herein or the oligonucleotide (e.g., PMO) conjugate disclosed herein. In some instances, the oligonucleotide conjugate disclosed herein comprises the binding moiety disclosed herein for delivering to a muscle cell conjugated to the oligonucleotide (e.g., PMO) molecule comprising a nucleic acid sequence of at least 25 consecutive nucleotides from one of SEQ ID NOs: 100-109. In some instances, the oligonucleotide (e.g., PMO) molecule has from about 24 to about 29 nucleotides, at least about 24 to about 28 nucleotides, at least about 24 to about 27 nucleotides, at least about 24 to about 26 nucleotides, at least about 24 to about 25 nucleotides, at least about 25 to about 29 nucleotides, at least about 25 to about 28 nucleotides, at least about 25 to about 27 nucleotides, at least about 25 to about 26 nucleotides, at least about 26 to about 29 nucleotides, at least about 26 to about 28 nucleotides, at least about 26 to about 27 nucleotides, at least about 27 to about 29 nucleotides, at least about 27 to about 28 nucleotides, at least about 28 to about 29 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, or at least 29 nucleotides in length. In some instances, the oligonucleotide (e.g., PMO) molecule consists of a sequence from one of SEQ ID NOs: 116-119. In some instances, the binding moiety binds to the cell surface molecule disclosed herein (e.g., a transferrin receptor) on the muscle cell. In some instances, the binding moiety is an anti-transferrin receptor antibody or antigen binding fragment thereof disclosed herein. In some instances, the subject in need thereof is diagnosed with or to have a high/higher chance to a disease or a disorder resulting from a mutation of the dystrophin gene that is amenable to exon 45 skipping. In some instances, the subject in need thereof is diagnosed with or to have a high/higher chance to a disease or a disorder resulting from a deletion leading to a premature stop codon in exon 45. In some instances, the subject in need thereof is diagnosed with or to have a high/higher chance to develop a muscular dystrophy. In some instances, the muscular dystrophy is Duchenne muscular dystrophy or Becker muscular dystrophy.

In some aspects, provided herein a method of generating a truncated dystrophin protein in a subject in need thereof comprising administering to the subject the oligonucleotide molecule (e.g., PMO) disclosed herein or the oligonucleotide (e.g., PMO) conjugate disclosed herein. In some instances, the oligonucleotide conjugate disclosed herein comprises the binding moiety disclosed herein for delivering to a muscle cell conjugated to the oligonucleotide (e.g., PMO) molecule comprising a nucleic acid sequence of at least 25 consecutive nucleotides from one of SEQ ID NOs: 100-109. In some instances, the oligonucleotide (e.g., PMO) molecule has from about 24 to about 29 nucleotides, at least about 24 to about 28 nucleotides, at least about 24 to about 27 nucleotides, at least about 24 to about 26 nucleotides, at least about 24 to about 25 nucleotides, at least about 25 to about 29 nucleotides, at least about 25 to about 28 nucleotides, at least about 25 to about 27 nucleotides, at least about 25 to about 26 nucleotides, at least about 26 to about 29 nucleotides, at least about 26 to about 28 nucleotides, at least about 26 to about 27 nucleotides, at least about 27 to about 29 nucleotides, at least about 27 to about 28 nucleotides, at least about 28 to about 29 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, or at least 29 nucleotides in length. In some instances, the oligonucleotide (e.g., PMO) molecule consists of a sequence from one of SEQ ID NOs: 116-119. In some instances, the binding moiety binds to the cell surface molecule disclosed herein (e.g., a transferrin receptor) on the muscle cell. In some instances, the binding moiety is an anti-transferrin receptor antibody or antigen binding fragment thereof disclosed herein. In some instances, the subject in need thereof is diagnosed with or to have a high/higher chance to a disease or a disorder resulting from a mutation of the dystrophin gene that is amenable to exon 45 skipping. In some instances, the subject in need thereof is diagnosed with or to have a high/higher chance to a disease or a disorder resulting from a deletion leading to a premature stop codon in exon 45. In some instances, the subject in need thereof is diagnosed with or to have a high/higher chance to develop a muscular dystrophy. In some instances, the muscular dystrophy is Duchenne muscular dystrophy or Becker muscular dystrophy.

In some aspects, provided herein a method of restoring dystrophin in a subject in need thereof comprising administering to the subject the oligonucleotide molecule (e.g., PMO) disclosed herein or the oligonucleotide (e.g., PMO) conjugate disclosed herein. In some instances, the oligonucleotide conjugate disclosed herein comprises the binding moiety disclosed herein for delivering to a muscle cell conjugated to the oligonucleotide (e.g., PMO) molecule comprising a nucleic acid sequence of at least 25 consecutive nucleotides from one of SEQ ID NOs: 100-109. In some instances, the oligonucleotide (e.g., PMO) molecule has from about 24 to about 29 nucleotides, at least about 24 to about 28 nucleotides, at least about 24 to about 27 nucleotides, at least about 24 to about 26 nucleotides, at least about 24 to about 25 nucleotides, at least about 25 to about 29 nucleotides, at least about 25 to about 28 nucleotides, at least about 25 to about 27 nucleotides, at least about 25 to about 26 nucleotides, at least about 26 to about 29 nucleotides, at least about 26 to about 28 nucleotides, at least about 26 to about 27 nucleotides, at least about 27 to about 29 nucleotides, at least about 27 to about 28 nucleotides, at least about 28 to about 29 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, or at least 29 nucleotides in length. In some instances, the oligonucleotide (e.g., PMO) molecule consists of a sequence from one of SEQ ID NOs: 116-119. In some instances, the binding moiety binds to the cell surface molecule disclosed herein (e.g., a transferrin receptor) on the muscle cell. In some instances, the binding moiety is an anti-transferrin receptor antibody or antigen binding fragment thereof disclosed herein. In some instances, the subject in need thereof is diagnosed with or to have a high/higher chance to a disease or a disorder resulting from a mutation of the dystrophin gene that is amenable to exon 45 skipping. In some instances, the subject in need thereof is diagnosed with or to have a high/higher chance to a disease or a disorder resulting from a deletion leading to a premature stop codon in exon 45. In some instances, the subject in need thereof is diagnosed with or to have a high/higher chance to develop a muscular dystrophy. In some instances, the muscular dystrophy is Duchenne muscular dystrophy or Becker muscular dystrophy.

In some aspects, provided herein a method of treating muscular dystrophy in a subject in need thereof comprising administering to the subject the oligonucleotide molecule (e.g., PMO) disclosed herein or the oligonucleotide (e.g., PMO) conjugate disclosed herein. In some instances, the oligonucleotide conjugate disclosed herein comprises the binding moiety disclosed herein for delivering to a muscle cell conjugated to the oligonucleotide (e.g., PMO) molecule comprising a nucleic acid sequence of at least 25 consecutive nucleotides from one of SEQ ID NOs: 100-109. In some instances, the oligonucleotide (e.g., PMO) molecule has from about 24 to about 29 nucleotides, at least about 24 to about 28 nucleotides, at least about 24 to about 27 nucleotides, at least about 24 to about 26 nucleotides, at least about 24 to about 25 nucleotides, at least about 25 to about 29 nucleotides, at least about 25 to about 28 nucleotides, at least about 25 to about 27 nucleotides, at least about 25 to about 26 nucleotides, at least about 26 to about 29 nucleotides, at least about 26 to about 28 nucleotides, at least about 26 to about 27 nucleotides, at least about 27 to about 29 nucleotides, at least about 27 to about 28 nucleotides, at least about 28 to about 29 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, or at least 29 nucleotides in length. In some instances, the oligonucleotide (e.g., PMO) molecule consists of a sequence from one of SEQ ID NOs: 116-119. In some instances, the binding moiety binds to the cell surface molecule disclosed herein (e.g., a transferrin receptor) on the muscle cell. In some instances, the binding moiety is an anti-transferrin receptor antibody or antigen binding fragment thereof disclosed herein. In some instances, the subject in need thereof is diagnosed with or to have a high/higher chance to a disease or a disorder resulting from a mutation of the dystrophin gene that is amenable to exon 45 skipping. In some instances, the subject in need thereof is diagnosed with or to have a high/higher chance to a disease or a disorder resulting from a deletion leading to a premature stop codon in exon 45. In some instances, the subject in need thereof is diagnosed with or to have a high/higher chance to develop a muscular dystrophy. In some instances, the muscular dystrophy is Duchenne muscular dystrophy or Becker muscular dystrophy.

In some aspects, provided herein a method of inducing exon 45 skipping in a targeted pre-mRNA transcript of DMD gene, comprising: 1) contacting a muscle cell with an oligonucleotide molecule or an oligonucleotide conjugate comprising a binding moiety for delivering to a muscle cell conjugated to the oligonucleotide molecule comprising a nucleic acid sequence of at least 25 consecutive nucleotides from one of SEQ ID NOs: 100-109; b) hybridizing the oligonucleotide molecule to the targeted pre-mRNA transcript to induce exon 45 skipping in the targeted pre-mRNA transcript; c) and translating a mRNA transcript produced from the targeted pre-mRNA transcript processed in step b) in the muscle cell to generate a truncated dystrophin protein. In some instances, the oligonucleotide conjugate disclosed herein comprises the binding moiety disclosed herein for delivering to a muscle cell conjugated to the oligonucleotide (e.g., PMO) molecule comprising a nucleic acid sequence of at least 25 consecutive nucleotides from one of SEQ ID NOs: 100-109. In some instances, the oligonucleotide (e.g., PMO) molecule has from about 24 to about 29 nucleotides, at least about 24 to about 28 nucleotides, at least about 24 to about 27 nucleotides, at least about 24 to about 26 nucleotides, at least about 24 to about 25 nucleotides, at least about 25 to about 29 nucleotides, at least about 25 to about 28 nucleotides, at least about 25 to about 27 nucleotides, at least about 25 to about 26 nucleotides, at least about 26 to about 29 nucleotides, at least about 26 to about 28 nucleotides, at least about 26 to about 27 nucleotides, at least about 27 to about 29 nucleotides, at least about 27 to about 28 nucleotides, at least about 28 to about 29 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, or at least 29 nucleotides in length. In some instances, the oligonucleotide (e.g., PMO) molecule consists of a sequence from one of SEQ ID NOs: 116-119. In some instances, the binding moiety binds to the cell surface molecule disclosed herein (e.g., a transferrin receptor) on the muscle cell. In some instances, In some instances, the binding moiety is an anti-transferrin receptor antibody or antigen binding fragment thereof disclosed herein.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, the terms “individual(s)”, “subject(s)” and “patient(s)” mean any mammal. In some aspects, the mammal is a human. In some aspects, the mammal is a non-human. None of the terms require or are limited to situations characterized by the supervision (e.g., constant or intermittent) of a health care worker (e.g., a doctor, a registered nurse, a nurse practitioner, a physician's assistant, an orderly or a hospice worker).

As used here, the term “DMD subject” means any mammal that suffers or expected to suffer from DMD, and/or has a genetic predisposition (e.g., mutations in DMD gene) related to the DMD. In some aspects, the mammal is a human. In some aspects, the mammal is a non-human.

As used here, the term “a subject affected by DMD” means any mammal that suffers or expected to suffer from DMD, and/or has a genetic predisposition (e.g., mutations in DMD gene) related to the DMD. In some aspects, the mammal is a human. In some aspects, the mammal is a non-human.

### List of Exemplary Embodiments

Embodiment 1: A phosphorodiamidate morpholino oligonucleotide (PMO) conjugate comprising an anti-transferrin receptor antibody or antigen binding fragment thereof conjugated to a PMO molecule that hybridizes to a pre-mRNA transcript of the DMD gene.

Embodiment 2: The PMO conjugate of embodiment 1, wherein the PMO molecule is selected from a group consisting of SEQ ID NOs:100-119.

Embodiment 3: The PMO conjugate of embodiment 1 or embodiment 2, wherein the PMO molecule hybridizes to an acceptor splice site, a donor splice site, or an exonic splice enhancer element of a pre-mRNA transcript of the DMD gene and induces exon 45 skipping in said pre-mRNA transcript to generate a mRNA transcript encoding a truncated dystrophin protein.

Embodiment 4: The PMO conjugate of embodiment 3, wherein the PMO molecule hybridizing to the exon 45 acceptor splice site of said pre-mRNA transcript of the DMD gene generates a mRNA transcript encoding a truncated dystrophin protein.

Embodiment 5: The PMO conjugate of any one of embodiments 1-4, wherein the PMO molecule comprises from about 10 to about 30 nucleotides in length.

Embodiment 6: The PMO conjugate of any one of embodiments 1-5, wherein the PMO molecule is delivered into a muscle cell.

Embodiment 7: The PMO conjugate of any one of embodiments 1-6, wherein the anti-transferrin receptor antibody or antigen binding fragment thereof comprises a humanized antibody or antigen binding fragment thereof, chimeric antibody or antigen binding fragment thereof, monoclonal antibody or antigen binding fragment thereof, monovalent Fab′, divalent Fab2, single chain variable fragment (scFv), diabody, minibody, nanobody, single domain antibody (sdAb), or camelid antibody or antigen binding fragment thereof.

Embodiment 8: The PMO conjugate of any one of embodiments 1-7, wherein the PMO molecule is conjugated to the anti-transferrin receptor antibody or antigen binding fragment thereof via a linker.

Embodiment 9: The PMO conjugate of embodiment 8, wherein the linker is a cleavable linker.

Embodiment 10: The PMO conjugate of 8, wherein the linker is a non-cleavable linker.

Embodiment 11: The PMO conjugate of embodiment 8, wherein the linker is selected from the group consisting of a heterobifunctional linker, a homobifunctional linker, a maleimide group, a dipeptide moiety, a benzoic acid group or derivatives thereof, a C1-C6 alkyl group, and a combination thereof.

Embodiment 12: The PMO conjugate of any one of embodiments 1-11, wherein the PMO conjugate has a PMO molecule to antibody ratio (DAR) of about 1:1, 2:1, 3:1, 4:1 5:1, 6:1, 7:1, 8:1 or higher.

Embodiment 13: The PMO conjugate of any one of embodiments 1-12, wherein the PMO conjugate has an average DAR of about 1, 2, 3, 4, 5, 6, 7, 8 or higher.

Embodiment 14: The PMO conjugate of any one of embodiments 1-13, wherein the PMO conjugate has an average DAR in the range of 4-5.

Embodiment 15: The PMO conjugate of any one of embodiments 1-13, wherein the PMO conjugate has an average DAR in the range of 7-8.

Embodiment 16: The PMO conjugate of any one of embodiments 1-13, wherein the PMO conjugate has an average DAR of about 4.

Embodiment 17: The PMO conjugate of any one of embodiments 1-13, wherein the PMO conjugate has an average DAR of about 8.

Embodiment 18: The PMO conjugate of any one of embodiments 1-13, wherein the PMO conjugate has a DAR of about 4.

Embodiment 19: The PMO conjugate of any one of embodiments 1-13, wherein the PMO conjugate has a DAR of about 8.

Embodiment 20: The PMO conjugate of any one of embodiments 1-19, wherein the PMO conjugate is formulated for parenteral administration.

Embodiment 21: The PMO conjugate of any one of embodiments 1-20, wherein the truncated dystrophin proteins modulate muscular dystrophy.

Embodiment 22: The PMO conjugate of embodiment 21, wherein the muscular dystrophy is Duchenne muscular dystrophy or Becker muscular dystrophy.

Embodiment 23: A method of treating muscular dystrophy in a subject in need thereof comprising administering to said subject a phosphorodiamidate morpholino oligonucleotide (PMO) conjugate comprising an anti-transferrin receptor antibody or antigen binding fragment thereof conjugated to a PMO molecule comprising a nucleic acid sequence selected from a group consisting of SEQ ID NOs:100-119; wherein the PMO molecule hybridizes to an acceptor splice site, a donor splice site, or an exonic splice enhancer element of a pre-mRNA transcript of the DMD gene and induces exon 45 skipping in said pre-mRNA transcript to generate a mRNA transcript encoding a truncated dystrophin protein.

Embodiment 24: The method of embodiment 23, wherein the PMO molecule is delivered into a muscle cell.

Embodiment 25: The method of embodiment 23 or embodiment 24, wherein the anti-transferrin receptor antibody or antigen binding fragment thereof comprises a humanized antibody or antigen binding fragment thereof, chimeric antibody or antigen binding fragment thereof, monoclonal antibody or antigen binding fragment thereof, monovalent Fab′, divalent Fab2, single chain variable fragment (scFv), diabody, minibody, nanobody, single domain antibody (sdAb), or camelid antibody or antigen binding fragment thereof.

Embodiment 26: The method of any one of embodiments 23-25, wherein the PMO molecule comprises from about 10 to about 30 nucleotides in length.

Embodiment 27: The method of any one of embodiments 23-26, wherein the PMO molecule is conjugated to the anti-transferrin receptor antibody or antigen binding fragment thereof via a linker.

Embodiment 28: The method of embodiment 27, wherein the linker is a cleavable linker.

Embodiment 29: The method of embodiment 27, wherein the linker is a non-cleavable linker.

Embodiment 30: The method of embodiment 27, wherein the linker is selected from the group consisting of a heterobifunctional linker, a homobifunctional linker, a maleimide group, a dipeptide moiety, a benzoic acid group or derivatives thereof, a C1-C6 alkyl group, and a combination thereof.

Embodiment 31: The method of any one of embodiments 23-30, wherein the PMO conjugate has an average of PMO molecule to antibody ratio (DAR) of about 1:1, 2:1, 3:1, 4:1, 5:1, 6:1, 7:1, or 8:1.

Embodiment 32: The method of any one of embodiments 23-31, wherein the PMO conjugate has an average DAR in the range of 4-5.

Embodiment 33: The method of any one of embodiments 23-31, wherein the PMO conjugate has an average DAR in the range of 7-8.

Embodiment 34: The method of any one of embodiments 23-31, wherein the PMO conjugate has an average DAR of about 4.

Embodiment 35: The method of any one of embodiments 23-31, wherein the PMO conjugate has an average DAR of about 8.

Embodiment 36: The method of any one of embodiments 23-31, wherein the PMO conjugate is administered parenterally.

Embodiment 37: The method of any one of embodiments 23-31, wherein the truncated dystrophin proteins modulate muscular dystrophy.

Embodiment 38: The method of embodiment 37, wherein the muscular dystrophy is Duchenne muscular dystrophy or Becker muscular dystrophy.

Embodiment 39: A method of inducing exon 45 skipping in a targeted pre-mRNA transcript of DMD gene, comprising:
a) contacting a muscle cell with a phosphorodiamidate morpholino oligonucleotide (PMO)-antibody conjugate, wherein the PMO-antibody conjugate comprises an anti-transferrin receptor antibody or antigen binding fragment thereof, and a PMO molecule targeting an acceptor splice site, a donor splice site, or an exonic splice enhancer element of the targeted pre-mRNA transcript of the DMD gene; wherein the PMO molecule induces exon 45 skipping in the targeted pre-mRNA transcript, and wherein the PMO-antibody conjugate is preferentially delivered into the muscle cell;
b) hybridizing the PMO molecule to the targeted pre-mRNA transcript to induce exon 45 skipping in the targeted pre-mRNA transcript; and
c) translating a mRNA transcript produced from the targeted pre-mRNA transcript processed in step b) in the muscle cell to generate a truncated dystrophin protein.

Embodiment 40: The method of embodiment 39, wherein the anti-transferrin receptor antibody or antigen binding fragment thereof comprises a humanized antibody or antigen binding fragment thereof, chimeric antibody or antigen binding fragment thereof, monoclonal antibody or antigen binding fragment thereof, monovalent Fab′, divalent Fab2, single chain variable fragment (scFv), diabody, minibody, nanobody, single-domain antibody (sdAb), or camelid antibody or antigen binding fragment thereof.

Embodiment 41: The method of embodiment 39 or embodiment 40, wherein the PMO molecule comprises at least from about 10 to about 30 nucleotides in length.

Embodiment 42: The method of any one of embodiments 39-41, wherein the PMO molecule comprises at least 90%, 95%, 99%, or 100% sequence identity to a nucleic acid sequence selected from a group consisting of SEQ ID NOs: 100-119.

Embodiment 43: The method of any one of embodiments 39-42, wherein the PMO molecule targets the acceptor site of exon 45.

Embodiment 44: The method of any one of embodiments 39-43, wherein the PMO molecule is conjugated to the anti-transferrin receptor antibody or antigen binding fragment thereof via a linker.

Embodiment 45: The method of embodiment 44, wherein the linker is a cleavable linker.

Embodiment 46: The method of embodiment 44, wherein the linker is a non-cleavable linker.

Embodiment 47: The method of embodiment 44, wherein the linker is selected from the group consisting of a heterobifunctional linker, a homobifunctional linker, a maleimide group, a dipeptide moiety, a benzoic acid group or derivatives thereof, a C1-C6 alkyl group, and a combination thereof.

Embodiment 48: The method of any one of embodiments 39-47, wherein the PMO conjugate has an average of PMO to antibody ratio (DAR) of about 1:1, 2:1, 3:1, 4:1, 5:1, 6:1, 7:1, 8:1 or higher.

Embodiment 49: The method of any one of embodiments 39-48, wherein the PMO conjugate has a DAR of about 1, 2, 3, 4, 5, 6, 7, 8 or higher.

Embodiment 50: The method of any one of embodiments 39-48, wherein the PMO conjugate has an average DAR in the range of 4-5.

Embodiment 51: The method of any one of embodiments 39-48, wherein the PMO conjugate has an average DAR in the range of 7-8.

Embodiment 52: The method of any one of embodiments 39-48, wherein the PMO conjugate has an average DAR of about 4.

Embodiment 53: The method of any one of embodiments 39-48, wherein the PMO conjugate has an average DAR of about 8.

Embodiment 54: The method of any one of embodiments 39-48, wherein the method is an in vivo method.

Embodiment 55: A method of inducing exon 45 skipping in a subject affected by DMD in need thereof comprising administering to said subject a phosphorodiamidate morpholino oligonucleotide (PMO) conjugate comprising an anti-transferrin receptor antibody or antigen binding fragment thereof conjugated to a PMO molecule comprising a nucleic acid sequence selected from a group consisting of SEQ ID NOs:100-119, wherein the PMO molecule hybridizes to the exon 45 acceptor splice site of a pre-mRNA transcript of the DMD gene and induces exon 45 skipping in said pre-mRNA transcript to generate a mRNA transcript encoding a truncated dystrophin protein.

Embodiment 56: A method of restoring dystrophin in a subject affected by DMD in need thereof comprising administering to said subject a phosphorodiamidate morpholino oligonucleotide (PMO) conjugate comprising an anti-transferrin receptor antibody or antigen binding fragment thereof conjugated to a PMO molecule comprising a nucleic acid sequence selected from a group consisting of SEQ ID NOs:100-119, wherein the PMO molecule hybridizes to the exon 45 acceptor splice site of a pre-mRNA transcript of the DMD gene and induces exon 45 skipping in said pre-mRNA transcript to generate a mRNA transcript encoding a truncated dystrophin protein.

Embodiment 57: A method of generating a truncated dystrophin protein in a subject affected by DMD in need thereof comprising administering to said subject a phosphorodiamidate morpholino oligonucleotide (PMO) conjugate comprising an anti-transferrin receptor antibody or antigen binding fragment thereof conjugated to a PMO molecule comprising a nucleic acid sequence selected from a group consisting of SEQ ID NOs:100-119, wherein the PMO molecule hybridizes to the exon 45 acceptor splice site of a pre-mRNA transcript of the DMD gene and induces exon 45 skipping in said pre-mRNA transcript to generate a mRNA transcript encoding a truncated dystrophin protein.

### EXAMPLES

These examples are provided for illustrative purposes only and not to limit the scope of the claims provided herein.

### Example 1: In Silico Identification of Phosphorodiamidate Morpholino Oligomers (PMOs) with Predicted High hDMD Exon 45 Skipping Activity

Several algorithms have been reported to identify regions on the human DMD (hDMD) pre-mRNA that would be amendable for exon 45 skipping activity. The PMO screening was focused on a specific region most proximal to the exon 45 acceptor site based on reported predictions for exon 45 skipping (Echigoya et al. 2015, PLoS ONE 10(3): e0120058).

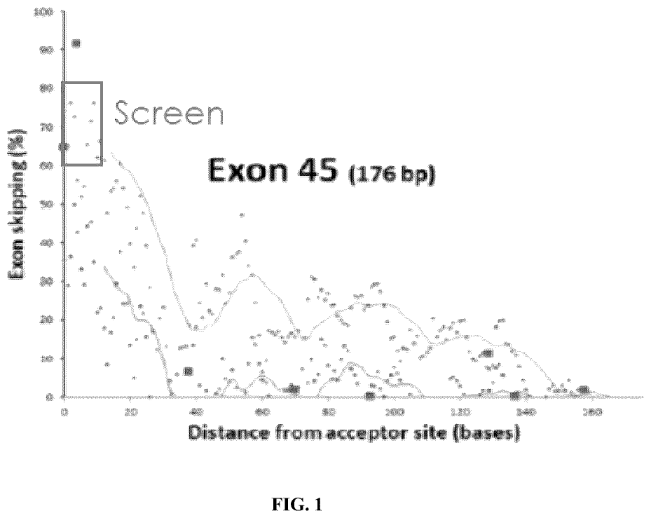

PMOs having hDMD exon 45 skipping activity were identified in silico. FIG. 1 shows exon 45 skipping activity of PMO 30-mers and 25-mers. The PMOs binding to the positions of the exon 45 acceptor site are labelled based on the distance (bases) of their 3′-end from the acceptor site. Large squares represent the exon 45 skipping activity of PMOs and dots represent PMOs with predicted exon 45 skipping activity. The acceptor site for exon 45 has a length of 176 base pairs (Source: NCBI Homo sapiens dystrophin (DMD), transcript variant Dp427m, mRNA. ACCESSION NM_004006): 5′-GAACTCCAGGATGGCATTGGGCAGCGGCAAACTGTTGTCAGAACATTGAATGCAAC TGGGGAAGAAATAATTCAGCAATCCTCAAAAACAGATGCCAGTATTCTACAGGAAA AATTGGGAAGCCTGAATCTGCGGTGGCAGGAGGTCTGCAAACAGCTGTCAGACAGA AAAAAGAG (SEQ ID NO: 200), and most active exon 45 skipping PMOs were observed to interact between the acceptor site at position 0 (Ac0) and the acceptor site at position 20 (Ac20). 10 PMOs (30-mers) with predicted hDMD exon 45 skipping activity greater than 60% (see box in FIG. 1) were further identified. The sequences of these 10 PMOs targeting the hDMD exon 45 acceptor sites are shown in Table 1.

### Results

Overall, 10 phosphorodiamidate morpholino oligomers (PMOs) with highest predicted exon 45 skipping activity were selected based on the algorithm that assist with the identification of regions on the hDMD pre-mRNA that can be amenable for exon 45 skipping activity.

### Example 2: Identification and Selection of PMO with High Exon 45 Skipping Activity in Healthy Primary Human Skeletal Muscle Cells (hSkMCs)

Selected 10 PMOs having high predicted exon 45 skipping activity from Example 1 were synthesized for additional in vitro assays in healthy primary human skeletal muscle cells (hSkMCs). SkMCs were obtained commercially (Gibco, #A11440). These cells were pre-differentiated and induced to form myotubes by plating on collagen Type 1 coated 24-well plates (Gibco, #1970788) (50000 cells/well) in DMEM supplemented with 2% horse serum and 1×ITS (Gibco, #1933286) for 2 days according to the manufacturer's instructions. PMOs were synthesized by GeneTools. PMOs were formulated in water, heated at 65-70° C. for 5 minutes, and diluted into warm medium together with 2 μM Endo-Porter (Gene Tools, #EP6P1-1) to facilitate PMO uptake into cells. Cells were harvested 48 hours post transfection. Cells were collected in Trizol and stored at −80° C. until processing for RNA isolation using Direct-zol-96 RNA isolation kit (Zymo) according to the manufacturer's instructions. Total RNA concentration was quantified spectroscopically. 100-200 ng of purified RNA was converted to cDNA using High-Capacity cDNA Reverse Transcription Kit (Applied Biosystems) and a SimpliAmp Thermal Cycler (Applied Biosystems). DNA fragments representing total DMD mRNA or exon 45-skipped DMD mRNAs were amplified by PCR using TaqMan Fast Advanced Master mix (Applied Biosystems) and either a hDMD TaqMan assay Hs01049401_m1 (VIC-MGB, Thermo Fisher Scientific) or a custom-made TaqMan assay specific for the hDMD exon 44/46 junction (FAM-MGB, Forward: 5′-GTTCTTCTAGCCTTAAGATACCATTTG-3′ (SEQ ID NO:80), Reverse: 5′-GTTCTTCTAGCCTTAAGATACCATTTG-3′ (SEQ ID NO:81), Probe: 5′-ACACAAATTCCTGAGAATTGGGAACATGC-3′) (SEQ ID NO:82)). For quantification of exon 45-skipped DMD levels by gel electrophoresis, PCR reactions were incubated at 95° C. for 20 seconds, followed by 32 cycles of 95° C. for 1 sec and 60° C. for 20 sec using a QuantStudio 7 Flex (Applied Biosystems). PCR products were diluted 4:1 with TAE loading buffer, loaded onto 24-well 4% TAE gels (Embi Tec, #GG3807) containing GelGreen. PCR products were separated by electrophoresis (50 V for 2 hours). The intensity of bands corresponding to total DMD and skipped DMD products were quantified by densitometry using ChemiDoc™ XRS+ (Bio-Rad).

The selected 10 PMOs were transfected in healthy primary hSkMCs that were pre-differentiated into myotubes using Endoporter as described above and harvested 48 hours post transfection. Total DMD mRNAs and exon 45 skipped DMD mRNAs were amplified by RT-qPCR. PCR products were separated by gel electrophoresis and quantified by densitometry. These results are from 2 independent experiments conducted with either 1, 3 and 10 μM of the PMOs in duplicates.

### Results

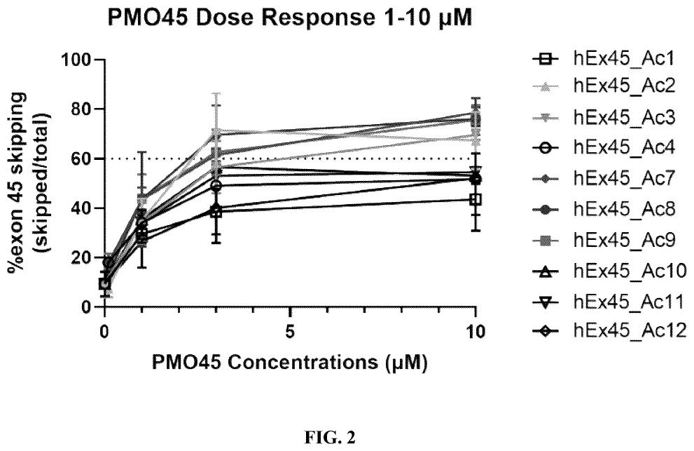

FIG. 2 shows the dose dependent response of the exon 45 skipping activity of the selected 10 PMOs (30-mers) targeting the acceptor site of human DMD exon 45 in healthy primary hSkMCs. The results of the assay in hSkMCs indicate that the maximal hDMD exon 45 skipping activity of the tested PMOs was greater than 60% confirming their predicted activities as shown in FIG. 1.

Overall, the in vitro assays for the activities of the selected 10 PMOs confirm their predicted exon 45 skipping activities.

### Example 3: Selection of Length-Optimized Exon 45 Skipping PMO in Healthy Primary Human Skeletal Muscle Cells (hSkMCs)

Primary human Skeletal Muscle Cells (hSkMCs) were obtained commercially (Gibco, #A11440). These cells were pre-differentiated and induced to form myotubes by plating on collagen Type 1 coated 24-well plates (Gibco, #1970788) (50000 cells/well) in DMEM supplemented with 2% horse serum and 1×ITS (Gibco, #1933286) for 2 days according to the manufacturer's instructions. PMOs were formulated in water, heated at 65-70° C. for 5 minutes, diluted into warm medium together with 2 μM Endo-Porter (Gene Tools, #EP6P1-1) to facilitate PMO uptake into cells. Cells were harvested 48 hours post-transfection. Cells were collected in Trizol and stored at −80° C. until processing for RNA isolation using Direct-zol-96 RNA isolation kit (Zymo) according to the manufacturer's instructions. Total RNA concentration was quantified spectroscopically. 100-200 ng of purified RNA was converted to cDNA using High-Capacity cDNA Reverse Transcription Kit (Applied Biosystems) and a SimpliAmp Thermal Cycler (Applied Biosystems). DNA fragments representing total or Exon 45-skipped DMD mRNAs were amplified by PCR using TaqMan Fast Advanced Master mix (Applied Biosystems) and either a hDMD TaqMan assay Hs01049401_m1 (VIC-MGB, Thermo Fisher Scientific) or a custom-made TaqMan assay specific for the hDMD exon 44/46 junction (FAM-MGB, Forward: 5′-CAGTGGCTAACAGAAGCTGA-3′(SEQ ID NO:83), Reverse: 5′-GTTCTTCTAGCCTTAAGATACCATTTG-3′(SEQ ID NO: 84), Probe: 5′-ACACAAATTCCTGAGAATTGGGAACATGC-3′ (SEQ ID NO:85)). For quantification of exon 45-skipped levels by gel electrophoresis, PCR reactions were incubated at 95° C. for 20 sec, followed by 32 cycles of 95° C. for 1 sec and 60° C. for 20 sec using a QuantStudio 7 Flex (Applied Biosystems). PCR products were diluted 4:1 with TAE loading buffer, loaded onto 24-well 4% TAE gels (Embi Tec, #GG3807) containing GelGreen. PCR products were separated by electrophoresis (50 V for 2 hrs). The intensity of bands corresponding to total DMD and skipped DMD products were quantified by densitometry using ChemiDoc™ XRS+ (Bio-Rad). The experiments were conducted with 1, 3, and 10 μM of PMO in healthy primary hSkMCs. PMOs were transfected using Endoporter as described below and harvested 48 hours post-transfection. Total and exon 45 skipped DMD mRNAs were monitored by RT-qPCR. PCR products were separated by gel electrophoresis and quantified by densitometry.

PMO oligonucleotides less than 30-mer length that achieved maximum activity were selected to lower manufacturing complexity and costs associated. Due to yield issues arising for the synthesis of PMO sequences that are greater than 30 nucleotides, several PMO sequences targeting the acceptor 9 site that range from 20 nucleotides to 29 nucleotides were designed as shown in Table 2. These PMOs were designed by removing 1 nucleotide at the 5′end at the time. The sequences of these 10 PMOs targeting the human DMD exon 45 acceptor 9 site are shown in Table 2. To identify the optimal length of the PMO sequence targeting acceptor site 9, 4 different PMO sequences targeting acceptor site 9 ranging from 20- to 28-mer in length were selected for further evaluation for a concentration dose response assay for skipping exon 45 in healthy primary hSkMCs.

### Results

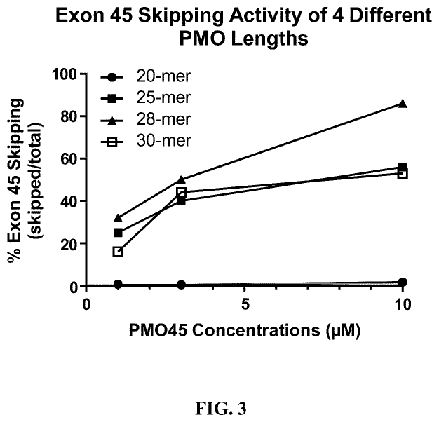

In order to select PMOs with an optimal length/activity ratio, exon 45 skipping activities in healthy primary hSkMCs transfected with different length of hEx45_Ac9 PMO sequences were evaluated (FIG. 3). The transfection of the selected PMO sequences into myotubes derived from healthy primary hSkMCs revealed that 3 different PMO sequences of various sizes have superior exon 45 skipping activities than that of the other ones. The 30-mer hEx45_Ac9_30 targeting the acceptor site 9, the 28-mer hEx45_Ac9_28 targeting the acceptor site 9, and the 25-mer hEx45_Ac9_25 targeting the acceptor site 9, showed at least 60% or greater exon 45 skipping activity at the concentration of 10 μM (FIG. 3). Among the 3 PMO sequences, the PMO hEx45_Ac9_28-mer displayed the best length/activity ratio and was selected as the PMO sequence for further evaluations for in vitro and in vivo assays.

Based on exon 45 skipping activities in myotubes derived from healthy primary hSkMCs, the 28-mer PMO hEx45_Ac9_28 (CAACAGTTTGCCGCTGCCCAATGCCATC (SEQ ID NO: 118) had the best exon 45 skipping activity among the 4 different PMO sequences. The hEx45_Ac9_28-mer was selected as the lead PMO sequence for further studies.

### Example 4: hEx45_Ac9_28 Induced Exon 45 Skipping Activities in Primary Healthy Cells and Primary DMD-Patient Derived Cells

Healthy and DMD primary human Skeletal Muscle Cells h(SkMCs) (primary DMD cells: 1479 (del44); Y537 (del46-55); AA355 (del18-44); healthy primary cells: W018; MB07; MB09) were obtained from the University of Rochester (NY), Centre de Resources Biologiques (CBC BioTec), France and from Besta Institute, Italy.

Primary cells were grown in skeletal muscle growth media (Promocell, Cat C-23160), 10 ng/ml EGF (Stemcell Technologies) and plated on 1% Matrigel coated 24-well plates (20000 cells/well). Myoblasts were induced to form myotubes in DMEM+Glutamax (Gibco) supplemented with Skeletal Muscle Cell Differentiation Medium Supplement Mix (Promocell, Cat C-39366) and 1% Pen/Strep for 2 days according to the manufacturer's instructions. PMOs were formulated in water, heated at 65-70° C. for 5-10 minutes, diluted into warm medium. Cells were harvested 48 h post transfection. Cells were collected in Trizol and stored at −80° C. until processing for RNA isolation using Direct-zol-96 RNA isolation kit (Zymo) according to the manufacturer's instructions. Total RNA concentration was quantified spectroscopically. cDNA was prepared from 200ng of purified RNA using the High-Capacity cDNA Reverse Transcription Kit (Applied Biosciences) in a SimpliAmp Thermal Cycler (Applied Biosystems). cDNA was partitioned into droplets, in triplicate, in the QX200 Automated Droplet Generator (BioRad) in combination with Taqman probes (Thermo Fisher), 2×ddPCR Supermix (no dUTP) (BioRad), and BamHI restriction enzyme (BioRad). Following droplet generation, the mixture was loaded into a deep well C1000 Touch Thermal Cycler (BioRad) for PCR amplification. Absolute quantification of the target RNA molecules was measured in the QX200 Droplet Digital PCR System (BioRad) using the QX Manager software (BioRad). Percent Exon Skipping was calculated by normalizing the counts of the targeted exon to the total gene expression. Primers used: hDMD TaqMan assay Hs01049401_m1 (VIC-MGB, Thermo Fisher Scientific) or a custom-made TaqMan assay specific for the hDMD exon 44/46 junction (FAM-MGB, Forward: 5′-AGATCTGTTGAGAAATGGC-3′ (SEQ ID NO: 86), Reverse: 5′-TGTTCTTCTAGCCTTAAGAT-3′ (SEQ ID NO: 87), Probe: 5′-TCAGTGGCTAACAGAAGCTGAACAGT-3′) (SEQ ID NO: 88), hDMD exon 43/46 junction (FAM-MGB, Forward: 5′-AGGGTGAAGCTACAGGAA-3′(SEQ ID NO: 89), Reverse: 5′-TTGTTCTTCTAGCCCTTGTC-3′ (SEQ ID NO: 90), Probe: 5′-TCTCTCCCAGCTTGATTTCCAATGGGA-3′) (SEQ ID NO: 91), hDMD exon 18/46 junction (FAM-MGB, Forward: 5′-GAGGCAGATTACTGTGGATTCTGA-3′ (SEQ ID NO: 303), Reverse: 5′-TGGTTGGAGGAAGCAGATAACA-3′ (SEQ ID NO: 92), Probe: 5′-TAGGAAAAGGCTAGAAGAA-3′ (SEQ ID NO: 93)), hDMD exon 44/56 junction (FAM-MGB, Forward: 5′-CACAAATTCCTGAGAATTGGGAAC-3′ (SEQ ID NO: 94), Reverse: 5′-TCATCCAGGTTGTGATAAACATCT-3′ (SEQ ID NO: 95), Probe: 5′-TGGTATCTTAAGGACCTCCAAGGTGA-3′(SEQ ID NO: 96)).

### Results

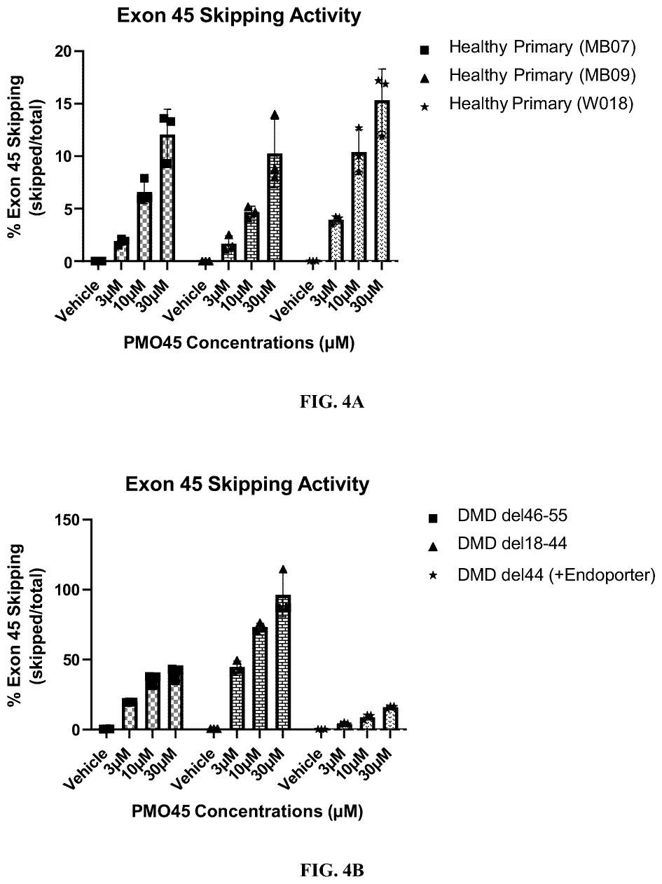

Exon 45 skipping activity was evaluated in primary healthy cells and primary DMD-patient derived cells using Digital droplet PCR (ddPCR), which is a sensitive and highly accurate method for exon skipping quantification. The hEx45_Ac9_28 PMO treatments were performed in myotubes derived from healthy human donors and DMD patients harboring deletion of exon 44, deletion of exons 46-55 or deletions of exons 18-44, which are all amenable to exon 45 skipping therapy (FIGS. 4A-B). A concentration-dependent exon 45 skipping activity with increasing concentrations of hEx45-Ac9-28 PMO was observed in 3 different healthy human cells (FIG. 4A) and in 3 different DMD patient-derived cells (FIG. 4B). Overall, the in vitro levels of exon 45 skipping activities of hEx45_Ac9_28 PMO were higher in all 3 DMD-patient derived cells compared to the levels of healthy cells. For the DMD-patient derived cells (DMD del44), a maximum of 15% of exon 45 skipping activity was measured in these human cells using Endoporter as transfection reagent (FIG. 4B).

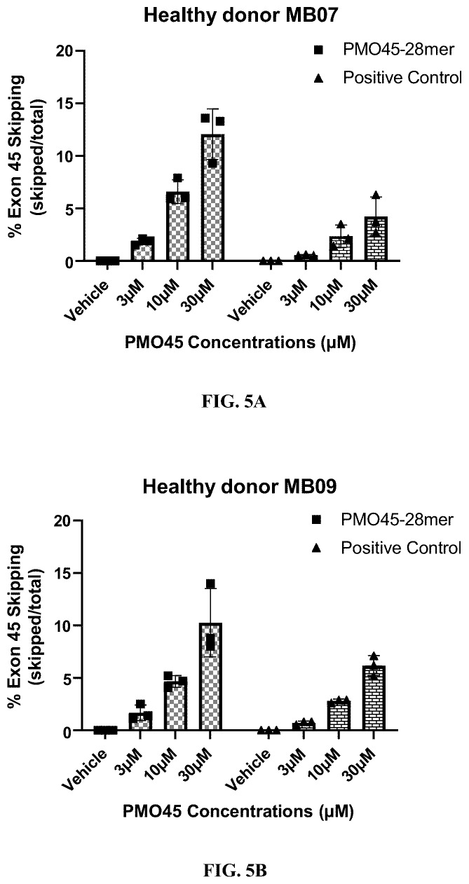

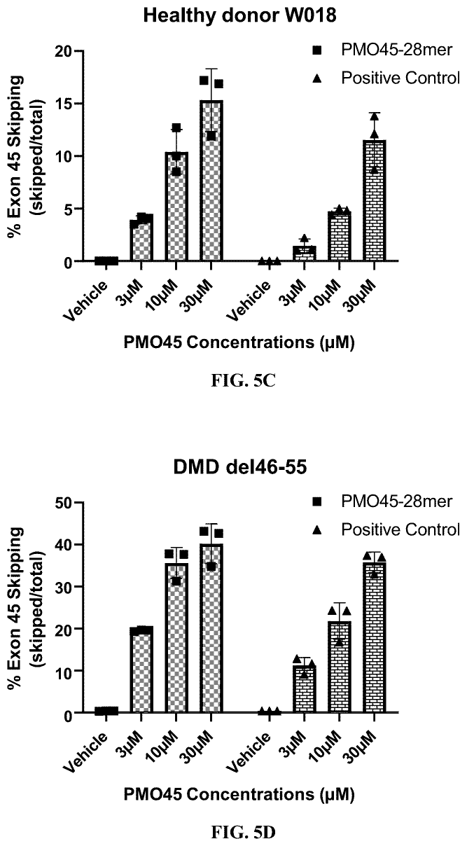

In addition, a comparison between the exon 45 skipping activities of the hEx45_Ac9_28 and a positive PMO control was evaluated in 3 primary healthy human cells and 3 primary DMD-patient derived cells. The positive control is an analog PMO to the PMO that has been approved by the FDA for the treatment of Duchenne muscular dystrophy (DMD) in patients who have a mutation in the DMD gene that is amenable to exon 45 skipping. The results indicate that the hEx45_Ac9_28 PMO showed greater exon 45 skipping activity levels compared to the positive PMO control in all 3 different healthy primary hSkMCs as measured by using ddPCR (FIGS. 5A-C). Moreover, the hEx45_Ac9_28 PMO showed greater exon 45 skipping activity levels than the levels of the positive control in all 3 different DMD primary hSkMCs as measured using ddPCR (FIGS. 5D-F).

Overall, the levels of exon 45 skipping activities for the hEx45_Ac9_28 PMO in DMD-patient derived cells were higher than those measured in healthy human cells, levels of exon 45 skipping activities of the hEx45_Ac9_28 PMO were higher than the levels measured with the positive PMO control.

### Example 5: hEx45_Ac9_28 Induces Dystrophin Restoration in DMD-Patient Derived Myotubes

Human myoblasts from DMD patients (I479, Y537, AA355) were amplified in Skeletal Muscle Growth medium (GM; Zenbio). At day 0, 15,000 of human primary myoblasts per well were seeded in GM in 96-well MyoScreen® plates (CYTOO) coated with 10 μg/ml fibronectin (Invitrogen). Induction of differentiation was performed after 24 hours of culture by changing the culture medium into differentiation medium (DM) composed of Dulbecco's Modified Eagle Medium: Nutrient Mixture F12 (DMEM/F12; Invitrogen), 2% horse serum (HS; GE Healthcare), 100 U/ml penicillin, and 100 μg/ml streptomycin (Invitrogen). At day 3, hEx45_Ac9_28 PMOs were added to the DMD myotubes and maintained until day 8 post-treatment. For PMO treatment, hEx45-Ac9-28 PMOs were heated for 5 minutes at 70° C., then cooled down slowly before addition to the medium. In this specific condition Endo-Porter (GeneTools) was added simultaneously to the wells at 1 μM as delivery reagent. For each experiment, a mock condition corresponding to vehicle+/−EndoPorter was included to be used as negative control. At day 9, myotubes were fixed with formalin and permeabilized for 15 minutes with 0.5% Triton in PBS. For immunofluorescence staining and dystrophin quantification, cells were blocked with 1% BSA, then incubated 2 hours at room temperature with primary antibodies prepared in blocking solution: myotubes were stained with a troponin-T specific antibody (ab45932 Abcam) or a myosin heavy chain specific antibody (14-6503-82 Thermo Fisher). N-terminal dystrophin was stained using NCL-DysB antibody (Leica). Cells were incubated for 2 hours at room temperature with the corresponding secondary antibodies in combination with Hoechst 33342 (Thermo Fisher Scientific, Courtaboeuf, France). Images of cells were acquired with the Operetta HCS platform (Perkin Elmer, Villebon sur Yvette, France) using a ×10 objective, with 11 fields acquired per well. Image processing and analysis were performed using dedicated algorithms developed on the Acapella High Content Imaging Software (Perkin Elmer, Villebon sur Yvette, France) by CYTOO. Myotube and nuclei segmentation was performed using the troponin-T or myosin heavy chain (MHC) staining intensity and the Hoechst staining. The segmentation threshold was selected to avoid the detection of background noise and eliminate aberrant structures, and myotube area and dystrophin mean intensity were calculated. Three healthy donors were included in each experiment (three primary myoblast cells from CYTOO-W018, X819, AA179). The mean intensity of dystrophin expression in these healthy myotubes was used as a reference.

### Results

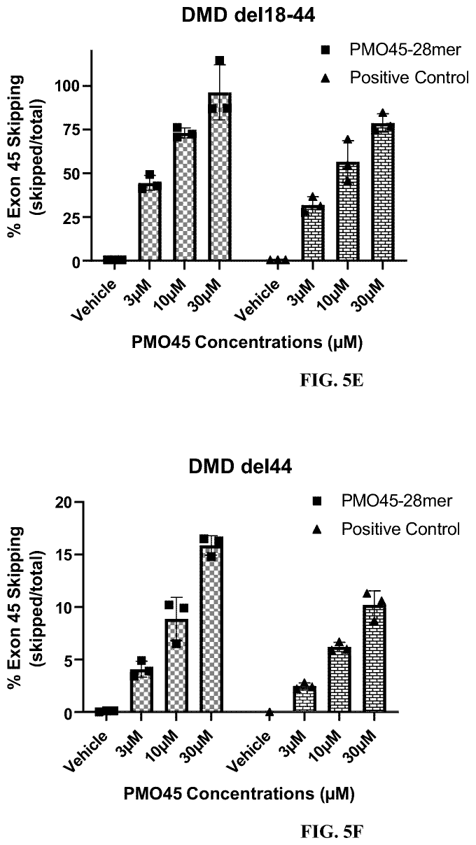

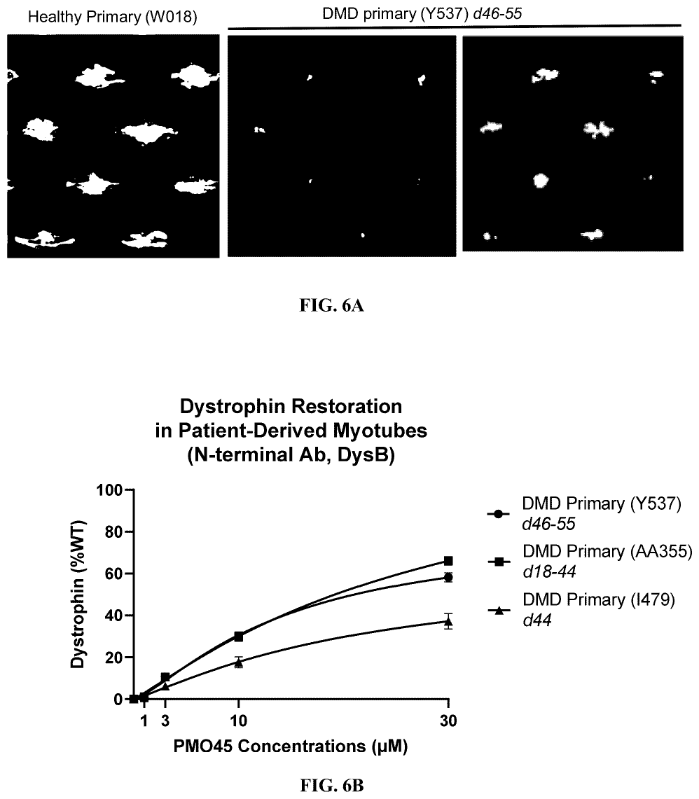

The dystrophin restoration assay was performed using the MyoScreen platform. This platform uses optimized culture conditions for differentiation, maturation, and longevity of cultured myotubes, and allows the quantification of dystrophin restoration by immunofluorescence. FIG. 6A shows pictures of healthy cells and primary DMD patient-derived cells (deletion of exon 46-55) on the MyoScreen platform that were immunofluorescently stained for dystrophin positive fibers. Healthy cells (left panel) and DMD patient derived cells transfected with hEx45_Ac9_28 (right panel) showed presence of dystrophin as indicated by the positive cellular immunofluorescence staining while the non-treated DMD patient derived cells did not express any dystrophin as evidenced by the lack of immunofluorescence staining (central panel).

The quantitative analysis of immunofluorescence staining for dystrophin restoration in DMD patient-derived cells transfected with increasing concentrations of hEx45_Ac9_28 PMO indicated that hEx45_Ac9_28 efficiently restored dystrophin in a dose-dependent manner in primary myotubes derived from 3 DMD patients that carry different deletions (exon 44 deletion, exons 18-44 deletion or exon 46-55 deletion)(FIG. 6B). At a concentration of 30 μM, the hEx45_Ac9_28 PMO was able to restore up 60% of dystrophin in primary DMD-patient derived cells compared to that of wild-type cells (healthy primary cells). Variation in the levels of dystrophin restoration may be dependent on the DMD donor cells. Therefore, treatment with hEx45_Ac9_28 PMO resulted in sufficient exon 45 skipping activity to produce increases in dystrophin protein levels in vitro.

Overall, the activity of the hEx45_Ac9_28 PMO was able to restore dystrophin levels in primary DMD-patient derived cells, and the degree of restoration of dystrophin levels was dependent on the DMD donor cells.

### Example 6: Biodistribution of Exon 45 Skipping DMD Copies in Muscle and Non-Muscle Tissues of Cynomolgus Monkey that have been Administered a Single Dose of DAR4 hEx45_Ac9_28 AOC at the Dose of 122.3 mg/kg at Day 0 Corresponding to the hEx45_Ac9_28 PMO Dose Level of 30 mg/kg

Scheme 1: Synthesis and Purification of DAR4 hEx45_Ac9_28 AOC

An anti-human transferrin receptor antibody was produced. The hEx45_Ac9_28 PMO was synthesized by GeneTools. Antibody (10 mg/ml) in borate buffer (25 mM sodium tetraborate, 25 mM NaCl, 1 mM Diethylene triamine pentaacetic acid, pH 8.0) was reduced by adding 4 equivalents of tris(2-carboxyethyl)phosphine (TCEP) in water and incubating at 37° C. for 4 hours. 4(N-maleimidomethyl)cyclohexanecarboxylic acid N-hydroxysuccinimide ester (SMCC) was coupled to the primary amine on the 3′ end of the hEx45_Ac9_28 PMO by incubating the hEx45_Ac9_28 PMO (50 mg/ml) in DMSO with 10 equivalents of SMCC (10 mg/ml) in DMSO for one hour. Unconjugated SMCC was removed by ultrafiltration using Amicon Ultra-15 centrifugal filter units with a MWCO of 3 kDa. The hEx45_Ac9_28 PMO-SMCC was washed three times with acetate buffer (10 mM sodium acetate, pH 6.0) and used immediately. The reduced antibody was mixed with 2.25 equivalents of hEx45_Ac9_28 PMO-SMCC and incubated overnight at 4° C. The pH of the reaction mixture was then reduced to 7.5 and 8 equivalents of N-Ethylmaleimide were added to the mixture at room temperature for 30 minutes to quench unreacted cysteines.

The reaction mixture was purified with an AKTA Explorer FPLC using HIC method-1. Dependent on the conjugate, fractions containing either conjugates with a drug to antibody ratio of one (DAR 1), two (DAR 2), three (DAR 3), four (DAR 4), five (DAR 5), six (DAR 6), seven (DAR 7), eight (DAR 8) or fractions containing conjugates with a drug to antibody ratio of 3+ (DAR 3+), 4+ (DAR 4+), 5+ (DAR 5+), 6+ (DAR 6+), 7+ (DAR 7+), (DAR 8+), or fractions containing either conjugates with an average drug to antibody ratio of one (DAR 1), two (DAR 2), three (DAR 3), four (DAR 4), five (DAR 5), six (DAR 6), seven (DAR 7), or eight (DAR 8) were combined and concentrated with Amicon Ultra-15 centrifugal filter units with a MWCO of 50 kDa. Concentrated conjugates were buffer exchanged with PBS (pH 7.4) using Amicon Ultra-15 centrifugal filter units prior to analysis.

### Hydrophobic Interaction Chromatography (HIC) Method-1

Column: GE, HiScreen Butyl HP, 4.7 ml
Solvent A: 50 mM phosphate buffer, 0.7M Ammonium Sulfate, pH 7.0; Solvent B: 80% 50 mM phosphate buffer, 20% IPA, pH 7.0; Flow Rate: 1.0 ml/min
Gradient:

| % A | % B | Column Volume |
|---|---|---|
| 100 | 0 | 1 |
| 70 | 30 | 25 |
| 0 | 100 | 1 |
| 0 | 100 | 2 |

hEx45_Ac9_28 AOC was quantified via BCA and analyzed by HIC (average DAR≈4.5), SEC (3.3% HMW), and LAL (<0.025 EU/mg anti-transferrin receptor antibody). The product was stored at 4° C.
   Scheme 2: Synthesis and Purification of DAR4 hEx45_Ac9_28 AOC

An anti-human transferrin receptor antibody was produced. The hEx45_Ac9_28 PMO was synthesized. Antibody (20.4 mg/ml) in citrate buffer (50 mM sodium citrate, 300 mM sucrose pH 6.5) was combined with ethylenediaminetetraacetic acid (EDTA, 0.5 M, 0.591 mL) and was reduced by adding 2 equivalents of tris(2-carboxyethyl)phosphine (TCEP) in water and incubating at 37° C. for 2 hours. 4(N-maleimidomethyl)cyclohexanecarboxylic acid N-hydroxysuccinimide ester (SMCC) was coupled to the primary amine on the 3′ end of the hEx45_Ac9_28 PMO by incubating the hEx45_Ac9_28 PMO (50 mg/ml) in 50 mM phosphate buffer pH 7.2 with 3 equivalents of SMCC (50 mg/ml) in DMSO for one hour. Unconjugated SMCC was removed by tangential flow filtration (TFF) with a membrane MWCO of 3 kDa with acetate buffer (10 mM sodium acetate, pH 6.0). The reduced antibody was mixed with 4.75 equivalents of hEx45_Ac9_28 PMO-SMCC and incubated for 1 hour at room temperature. N-Ethylmaleimide (10 equivalents, 15 mg/ml in DMSO, 25 mg) was added to the mixture at room temperature for 30 minutes to quench unreacted cysteines. The reaction was diluted to 1 L with endotoxin free water. Excess PMO and NEM were removed via SCX purification (GE SP/HP 16 10 resin) using SCX method-1. The combined fractions were buffer exchanged via TFF into citrate buffer (50 mM sodium citrate, 60 mM NaCl, pH 5.5) and concentrated to approximately 25 mg Ab/mL. The solution was sterile filtered with a 0.22 μm membrane.

### Strong Cation Chromatography (SCX) Method-1

Column: GE HiScale 50, HiPrep SP HP, 200 ml
Solvent A: 25 mM acetate, 25 mM PB, pH 6; Solvent B: 25 mM acetate, 25 mM PB, pH 6, 0.5 mM NaCl; Flow Rate: 30 ml/min
Gradient:

| % A | % B | Column Volume |
|---|---|---|
| 100 | 0 | 3 |
| 40 | 60 | 1.5 |
| 0 | 100 | 0.2 |
| 0 | 100 | 1 |

hEx45_Ac9_28 AOC was quantified via BCA and analyzed by HIC (average DAR 4.5), SEC (3.3% HMW), and LAL (<0.025 EU/mg anti-transferrin receptor antibody). The product was stored at 4° C.

### PMO Tissue Concentrations Assay

Cynomolgus monkeys received a single intravenous (IV) infusion of hEx45_Ac9_28 AOC at 122.3 mg/kg, which corresponded to a PMO dose level of 30 mg/kg. Muscle tissue and non-muscle biopsy samples were obtained from the cynomolgus monkeys on day 42 (prior to necropsy). 3 female animals per group were analyzed.

A hybridization-based assay was used to quantify total hEx_45_Ac9_28 PMO concentration in tissue. Tissue samples of 25-45 mg were homogenized in RIPA buffer on an OMNI Bead Ruptor Elite. Calibration standards for an 8-point standard curve were generated by serial dilution of DAR8 hEx45_Ac9_28 AOC into pooled homogenate of pre-dose gastrocnemius and vastus lateralis samples. DAR8 hEx45_Ac9_28 AOC standards and study samples were digested with 200 μg/mL proteinase K overnight at 60° C. A final round of homogenization was performed following digestion. The calibration standards and study samples were diluted similarly in assay diluent to ensure that sample values fell within the linear range of the standard curve and then incubated in 5 nM hEx_45_Ac9_28 DNA probe in hybridization buffer to allow for hybridization of hEx_45_Ac9_28 to the probe. MSD assay plates were washed and blocked, then hybridized samples were added to the assay plates. The plates were sealed and incubated to allow biotin on the probe to bind to streptavidin in the well. Plates were washed, then 6 U/mL MNase in MNase buffer was added to each well and incubated to remove un-hybridized probe from the plate. Plates were washed and detection buffer containing 0.5 μg/mL Ruthenylated detection antibody was added to each well, followed by incubation. Plates were washed, then read buffer was added to each well, followed by immediate reading on MSD Sector S 600 plate reader. The standard curve of ECL units vs. log base 10 of corresponding hEx_45_Ac9_28 PMO concentrations was generated in GraphPad Prism and fitted using a 5-parameter logistic (5-PL) fit equation (with 1/y² weighting). Study sample concentrations were interpolated from the standard curve equation and normalized based on tissue weight for tissue concentration calculation. The lower limit of quantification is 4.83 ng/mL or 0.51 nM.

### Results

Cynomolgus monkeys received a single IV infusion of hEx45_Ac9_28 AOC at Day 0 at the dose of 122.3 mg/kg at Day 0 corresponded to the PMO (hEx45_Ac9_28) dose level of 30 mg/kg. The amount of hEx45_Ac9_28 PMO delivered to the tissues was measured by a hybridization assay. Mean total hEx_Ac9_28 PMO concentrations were evaluated in tissue samples obtained from NHP at Day 42 post dose (FIG. 7). The highest hEx45_Ac9_28 PMO tissue concentrations were observed in non-muscle tissues that include the kidney, adrenal gland, and liver, followed by muscle tissues that include the heart, aorta, gastroenemius muscle, diaphragm, biceps brachii, vastus lateralis muscle, and intercostal muscle.

Overall, the hEx45_Ac9_28 AOC delivers the hEx45_Ac9_28 PMO to muscle and non-muscle tissues of animals.

### Example 7: Exon 45 Skipping Activities in Muscle Tissues Obtained from Cynomolgus Monkeys Received a Single Intravenous (IV) Infusion of hEx45_Ac9_28 AOC at 122.3 mg/kg

The synthesis and purification of hEx45_Ac9_28 AOC are shown in Scheme 1 and scheme 2 in Example 6.

### Exon 45 Skipping Assay

Cynomolgus monkeys received a single intravenous (IV) infusion of hEx45_Ac9_28 AOC at 122.3 mg/kg, which corresponded to a PMO dose level of 30 mg/kg. Muscle tissue and non-muscle biopsy samples were obtained from the cynomolgus monkeys on day 42 (prior to necropsy). 3 female animals per group were analyzed.

Cynomolgus monkey muscle and non-muscle tissue samples ranging from 20-50 mg were homogenized in 1 mL of TRIzol (Thermo Fisher) on the OMNI Bead Ruptor Elite system (OMNI International). RNA was isolated from tissue homogenate supernatant using the Direct-zol-96 RNA kit (Zymo Research) according to the manufacturer's instructions. 250 ng of purified RNA was converted to cDNA using the High-Capacity cDNA Reverse Transcription Kit (Applied Biosystems) and SimpliAmp Thermal Cycler (Applied Biosystems). ddPCR was performed on 50 ng of cDNA in a reaction containing the commercially available Total DMD Taqman Assay (Mf01049436_m1 VIC-MGB, Thermo Fisher), custom Skipped DMD Taqman Assay (, Thermo Fisher), ddPCR Supermix for probes (no dUTP, Bio-Rad), BamHI-HF restriction enzyme (New England BioLabs), and Ambion nuclease free water (Thermo Fisher). Each sample, run in triplicates, was partitioned into droplets in the QX200 Automated Droplet Generator (Bio-Rad). Following droplet generation, samples were transferred to a C1000 Touch Thermal Cycler with 96-Deep Well Reaction Module (Bio-Rad). After PCR amplification, samples were loaded into the QX200 Droplet Reader (Bio-Rad). Data were analyzed using QX Manager Software, Standard Edition, Version 1.2 (Bio-Rad). Discrimination between positive and negative droplets was achieved by manually applying a fluorescence amplitude threshold. Percent exon skipping was calculated as 100*(number of skipped exon 45 DMD copies per Total DMD copies per ng of cDNA).

### Results

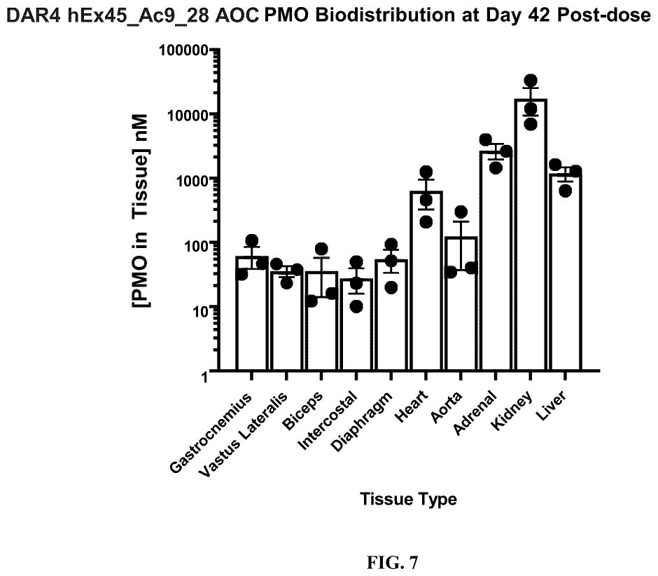

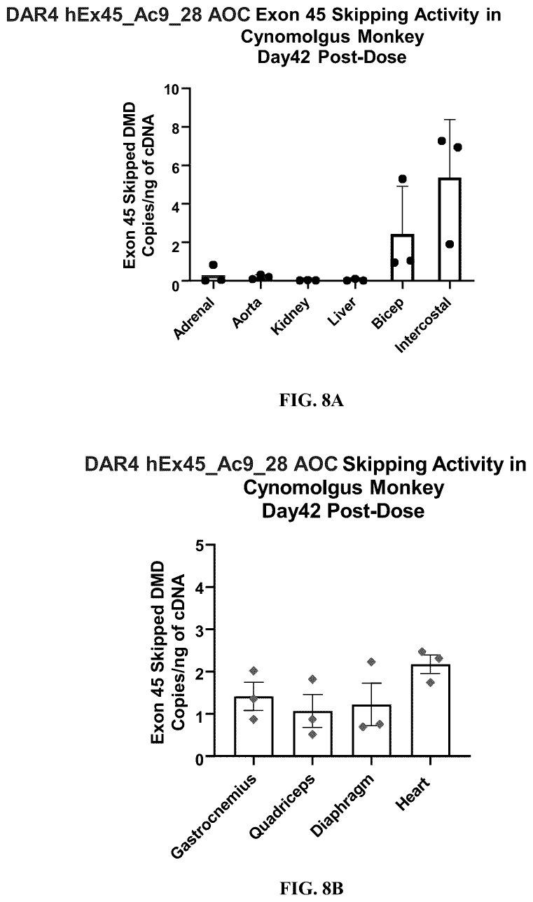

Muscle and non-muscle tissues from the animals were collected at 42 days post-dose and the number of exon 45 skipped copies in these tissues was measured by ddPCR. After 42 days post IV infusion of a single dose of hEx45_Ac9_28 AOC at 122.3 mg/kg, exon 45 skipping activity was detected in all muscle tissues, but it was not detected in non-muscle tissues, including the liver and kidney tissues (FIG. 8A). Exon 45 skipping activities in muscles were detected in skeletal muscles as well as cardiac muscles (FIG. 8B). The highest level of exon 45 skipping activity in muscle tissue was measured in the rectus femoris muscle (quadriceps) with greater numbers of exon 45 skipped copies per ng of cDNA, and the lowest one was measured in the biceps measured by exon 45 skipped copies per ng of cDNA. In addition, exon 45 skipping activities in the atria and ventricles of the heart were measured with an average of approximately 2 exon 45 skipped copies per ng of cDNA. These results indicate that hEx45_Ac9_28 AOC specifically targets muscle tissues, both skeletal and cardiac muscles, and induces exon 45 skipping in these specific tissues. Exon 45 skipping activities were detected 42 days after the single IV infusion of hEx45_Ac9_28 AOC confirming its long-lasting activity in the targeted tissues. Overall, exon 45 skipping activity induced by hEx45_Ac9_28 AOC is specific to muscle tissues, and the hEx45_Ac9_28 AOC has long lasting exon 45 skipping activities in muscle tissues.

### Example 8: hEx45_Ac9_28 PMO Tissue Concentrations in Vastus Lateralis and Gastrocnemius of Cynomolgus Monkey that have been Administered a Single Dose of DAR4 hEx45_Ac9_28 AOC at the Dose of 122.3 mg/kg at Day 0 Corresponding to the PMO (hEx45_Ac9_28) Dose Level of 30 mg/kg or DAR8 hEx45_Ac9_28 AOC at the Dose of 93.3 Kg/Mg at Day 0 Corresponding to the PMO (hEx45_Ac9_28) Dose Level of 30 mg/kg

The synthesis and purification of DAR4 hEx45_Ac9_28 AOC are shown in Scheme 1 and scheme 2 in Example 6.

Synthesis and Purification of DAR8 hEx45_Ac9_28 AOC

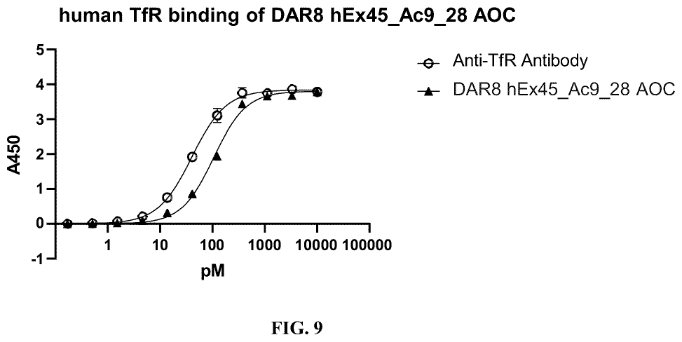

An anti-human transferrin receptor (anti-TfR) antibody was produced in citrate buffer (50 mM sodium citrate, 300 mM sucrose pH 6.5). The hEx45_Ac9_28 PMO-SMCC was synthesized by coupling 4(N-Maleimidomethyl)cyclohexanecarboxylic acid N-hydroxysuccinimide ester (SMCC) to the primary amine on the 3′ end of the hEx45_Ac9_28 PMO (PMO45). Ethylenediaminetetraacetic acid (EDTA, 0.5 M, 0.05 mL) was added to the antibody and the solution was thoroughly mixed. The antibody was reduced by the addition of tris(2-carboxyethyl)phosphine (TCEP, 10 mg/mL in water, 6 equiv., 37.75 mg, 1.89 mL). The antibody solution was incubated at 37° C. for 2 hours. The reduced antibody solution was removed from the incubator, cooled to room temperature, then diluted with 20 mM histidine pH 6 buffer (161.76 mL). The reduced antibody solution was combined with SMCC-PMO45 (Ajinomoto) solution (9.75 equiv., 60 mg/mL in DMSO, 34.47 mL) and mixed thoroughly. The reaction was incubated at room temperature for 1 hour. NEM solution (10 equiv., 25 mg/mL in DMSO, 27.5 mg, 1.1 mL) was added to the reaction mixture, mixed thoroughly, and the solution let sit for 30 minutes at room temperature. The reaction mixture was diluted to 0.7 L with 20 mM Histidine pH 6.0. Excess PMO and NEM were removed via SCX purification (GE SP/HP 16/−10 resin). The combined fractions were buffer exchanged via TFF into 20 mM histidine, 10 mM methionine, 120 mM sucrose, pH 6 and concentrated to approximately 25 mg mAb/mL. The solution was sterile filtered with a 0.22 um membrane. PMO45-AOC was quantified via BCA (23.75 mg of mAb/mL). PS80 solution (stock 10%) was added to the PMO45-AOC solution to bring the final PS80 concentration to 0.03% v/v. The final sample was analyzed by HIC (avg DAR≈7.7), SEC (2.9% HMW), and LAL (0.007 EU/mg mAb). The product was stored at 4° C. (2.4 g anti-TfR antibody, 73% yield). The affinity of the DAR8 hEx45_Ac9_28 AOC was equivalent to unconjugated anti-transferrin receptor antibody as shown in FIG. 9.

### PMO Tissue Concentrations Assay

Cynomolgus monkeys received a single intravenous (IV) infusion of DAR4 hEx45_Ac9_28 AOC at 122.3 mg/kg, which corresponded to a PMO dose level of 30 mg/kg, or DAR8 hEx45_Ac9_28 AOC at 93.3 mg/kg, which corresponded to a PMO level of 30 mg/kg. Muscle tissue biopsy samples were obtained from the cynomolgus monkeys on day 7 by punch muscle biopsy. 3 female animals per group were analyzed.

A hybridization-based assay was used to quantify total hEx_45_Ac9_28 PMO concentration in tissue. Tissue samples of 25-45 mg were homogenized in RIPA buffer on an OMNI Bead Ruptor Elite. Calibration standards for an 8-point standard curve were generated by serial dilution of DAR8 hEx45_Ac9_28 AOC into pooled homogenate of pre-dose gastrocnemius and vastus lateralis samples. DAR8 hEx45_Ac9_28 AOC standards and study samples were digested with 200 μg/mL proteinase K overnight at 60° C. A final round of homogenization was performed following digestion. The calibration standards and study samples were diluted similarly in assay diluent to ensure that sample values fell within the linear range of the standard curve and then incubated in 5 nM hEx_45_Ac9_28 DNA probe in hybridization buffer to allow for hybridization of hEx_45_Ac9_28 to the probe. MSD assay plates were washed and blocked, then hybridized samples were added to the assay plates. The plates were sealed and incubated to allow biotin on the probe to bind to streptavidin in the well. Plates were washed, then 6 U/mL MNase in MNase buffer was added to each well and incubated to remove un-hybridized probe from the plate. Plates were washed and detection buffer containing 0.5 μg/mL Ruthenylated detection antibody was added to each well, followed by incubation. Plates were washed, then read buffer was added to each well, followed by immediate reading on MSD Sector S 600 plate reader. The standard curve of ECL units vs. log base 10 of corresponding hEx_45_Ac9_28 PMO concentrations was generated in GraphPad Prism and fitted using a 5-parameter logistic (5-PL) fit equation (with 1/y² weighting). Study sample concentrations were interpolated from the standard curve equation and normalized based on tissue weight for tissue concentration calculation. The lower limit of quantification is 4.83 ng/mL or 0.51 nM.

### Results

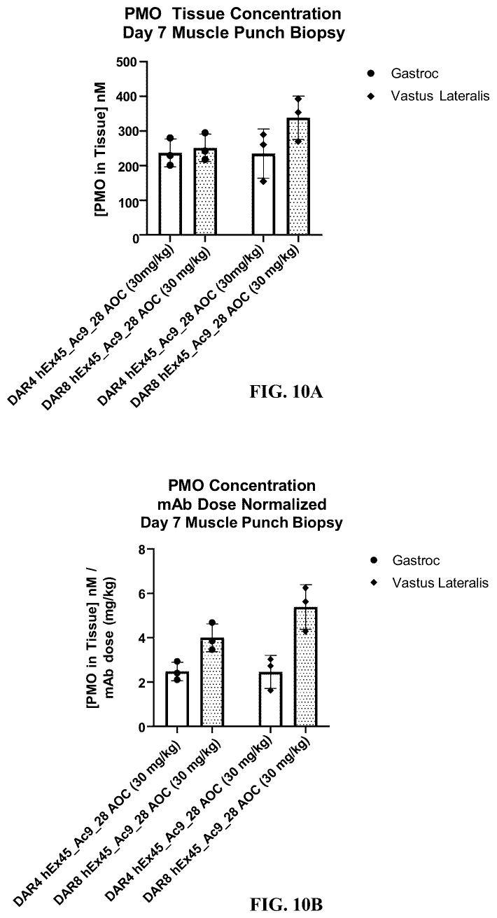

Animals received a single dose of DAR4 hEx45_Ac9_28 AOC or DAR8 hEx45_Ac9_28 AOC via a 60-minute intravenous (IV) infusion. The AOC dose levels of 122.3 mg/kg for the DAR4 hEx45_Ac9_28 AOC corresponded to 30 mg/kg of PMO component and the AOC dose levels of 93.3 mg/kg for the DAR8 hEx45_Ac9_28 AOC corresponded to 30 mg/kg of PMO component. At day 7 post-dose, hEx_45_Ac9_28 PMO tissue concentrations were measured in samples taken from the skeletal muscles, gastrocnemius and vastus lateralis muscles, obtained from the animals (FIG. 10A). The results indicate that hEx_45_Ac9_28 PMO tissue concentrations were approximately 220 nM in gastrocnemius muscles obtained from animals administered with DAR4 hEx45_Ac9_28 AOC or DAR8 hEx45_Ac9_28 AOC. In addition, hEx_45_Ac9_28 PMO tissue concentrations in vastus lateralis were higher in animals administered with DAR8 hEx45_Ac9_28 AOC (320 nM) than the animals administered with DAR4 hEx45_Ac9_28 AOC (220 nM). When the hEx_45_Ac9_28 PMO tissue concentrations were normalized to the doses of anti-TfR antibody administered, the hEx_45_Ac9_28 PMO tissue concentrations measured in the gastrocnemius or vastus lateralis are least 2-fold higher for the animals administered with DAR8 hEx45_Ac9_28 AOC than the concentrations for the animals administered with DAR4 hEx45_Ac9_28 AOC (FIG. 10B). The antibody normalized hEx 45 Ac9 28 concentrations of 4 nM for DAR8 hEx45 Ac9 28 AOC is double the hEx_45_Ac9_28 concentrations of 2 nM for DAR4 hEx45_Ac9_28 AOC in the gastrocnemius. Similarly, the antibody normalized hEx_45_Ac9_28 concentrations of 5.5 nM for DAR8 hEx45_Ac9_28 AOC is 2-fold higher the hEx_45_Ac9_28 concentrations of 2.2 nM for DAR4 hEx45_Ac9_28 AOC in the vastus lateralis. The antibody normalized hEx_45_Ac9_28 PMO concentrations indicate that the DAR8 hEx45 Ac9 28 AOC with DAR8 is able to deliver to muscles twice as much hEx_45_Ac9_28 PMO as the DAR4 hEx45_Ac9_28 AOC with DAR4.

Overall, the hEx_45_Ac9_28 PMO tissue concentrations measured in skeletal muscles indicate that both DAR4 hEx45_Ac9_28 AOC and DAR8 hEx45_Ac9_28 AOC can successfully deliver hEx45_Ac9_28 PMO to skeletal muscles. More importantly, the DAR8 hEx45_Ac9_28 AOC can deliver 2-fold or more hEx45_Ac9_28 PMO to muscles than DAR4 hEx45_Ac9_28 AOC.

While preferred aspects of the present disclosure have been shown and described herein, it will be obvious to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby.

What is claimed is:

1. A phosphorodiamidate morpholino oligonucleotide (PMO) conjugate comprising an anti-transferrin receptor antibody or antigen binding fragment thereof conjugated to a PMO molecule, wherein the PMO molecule comprises a nucleic acid sequence of at least 25 consecutive nucleotides from one of SEQ ID NOs: 100-109, and wherein the PMO molecule has 26-29 nucleotides in length.

2. The PMO conjugate of claim 1, wherein the PMO molecule consists of a sequence from one of SEQ ID NOs: 116-119.

3. The PMO conjugate of claim 1, wherein the anti-transferrin receptor antibody or antigen binding fragment thereof comprises a humanized antibody or antigen binding fragment thereof, chimeric antibody or antigen binding fragment thereof, monoclonal antibody or antigen binding fragment thereof, monovalent Fab′, divalent Fab2, single chain variable fragment (scFv), diabody, minibody, nanobody, single domain antibody (sdAb), or camelid antibody or antigen binding fragment thereof.

4. The PMO conjugate of claim 1, wherein the PMO molecule is conjugated to the anti-transferrin receptor antibody or antigen binding fragment thereof via a linker.

5. The PMO conjugate of claim 8, wherein the linker is a cleavable linker, a non-cleavable linker, or is selected from the group consisting of a heterobifunctional linker, a homobifunctional linker, a maleimide group, a dipeptide moiety, a benzoic acid group or derivatives thereof, a C1-C6 alkyl group, and a combination thereof.

6. The PMO conjugate of claim 1, wherein the PMO conjugate has a drug-antibody ratio (DAR) of about 1:1, 2:1, 3:1, 4:1 5:1, 6:1, 7:1, 8:1 or higher or wherein the PMO conjugate has an average DAR of about 1, 2, 3, 4, 5, 6, 7, 8 or higher.

7. The PMO conjugate of claim 10, wherein the PMO conjugate has a DAR of about 4:1 to 5:1, or wherein the PMO conjugate has an average DAR in the range of 4-5.

8. The PMO conjugate of claim 10, wherein the PMO conjugate has a DAR of about 7:1 to 8:1, or wherein the PMO conjugate has an average DAR in the range of 7-8.

9. The PMO conjugate of claim 10, wherein the PMO conjugate has a DAR of about 4:1 or 8:1 or wherein the PMO conjugate has an average DAR ratio of about 4 or 8.

10. A phosphorodiamidate morpholino oligonucleotide (PMO) molecule that hybridizes to a pre-mRNA transcript of a DMD gene, wherein the PMO molecule comprises a nucleic acid sequence of at least 25 consecutive nucleotides from one of SEQ ID NOs: 100-109, and wherein the PMO molecule has 26-29 nucleotides in length.

11. The PMO molecule of claim 10, wherein the PMO molecule consists of a sequence from one of SEQ ID NOs: 116-119.

12. A method of treating muscular dystrophy in a subject in need thereof comprising administering to the subject a phosphorodiamidate morpholino oligonucleotide (PMO) molecule or a PMO conjugate comprising an anti-transferrin receptor antibody or antigen binding fragment thereof conjugated to the PMO molecule, wherein the PMO molecule comprises a nucleic acid sequence of at least 25 consecutive nucleotides from one of SEQ ID NOs: 100-109, and wherein the PMO molecule has 26-29 nucleotides in length.

13. The method of claim 12, wherein the PMO molecule consists of a sequence from one of SEQ ID NOs: 116-119.

14. The method of claim 12, wherein the anti-transferrin receptor antibody or antigen binding fragment thereof comprises a humanized antibody or antigen binding fragment thereof, chimeric antibody or antigen binding fragment thereof, monoclonal antibody or antigen binding fragment thereof, monovalent Fab′, divalent Fab2, single chain variable fragment (scFv), diabody, minibody, nanobody, single domain antibody (sdAb), or camelid antibody or antigen binding fragment thereof.

15. The method of claim 12, wherein the PMO molecule is conjugated to the anti-transferrin receptor antibody or antigen binding fragment thereof via a linker.

16. The method of claim 15, wherein the linker is a cleavable linker, a non-cleavable linker, or is selected from the group consisting of a heterobifunctional linker, a homobifunctional linker, a maleimide group, a dipeptide moiety, a benzoic acid group or derivatives thereof, a C1-C6 alkyl group, and a combination thereof.

17. The method of claim 12, wherein the PMO conjugate has a drug-antibody ratio (DAR) of about 1:1, 2:1, 3:1, 4:1 5:1, 6:1, 7:1, 8:1 or higher or wherein the PMO conjugate has an average DAR of about 1, 2, 3, 4, 5, 6, 7, 8 or higher.

18. The method of claim 17, wherein the PMO conjugate has a DAR of about 4:1 to 5:1, or wherein the PMO conjugate has an average DAR in the range of 4-5.

19. The method of claim 17, wherein the PMO conjugate has a DAR of about 7:1 to 8:1, or wherein the PMO conjugate has an average DAR in the range of 7-8.

20. The method of claim 17, wherein the PMO conjugate has a DAR of about 4:1 or 8:1 or wherein the PMO conjugate has an average DAR ratio of about 4 or 8.

21. The method of claim 17, wherein the subject in need thereof is diagnosed with or to have a high/higher chance to develop a muscular dystrophy.

22. The method of claim 21, wherein the muscular dystrophy is Duchenne muscular dystrophy or Becker muscular dystrophy.
